(19) United States
(12) Patent Application Publication
(10) Pub. No.: US 2024/0299163 A1
(43) Pub. Date: Sep. 12, 2024

(54) SYSTEMS AND METHODS FOR IMPLANT DEPLOYMENT

(71) Applicant: EDWARDS LIFESCIENCES CORPORATION, Irvine, CA (US)

(72) Inventors: Taylor Jacob Scheinblum, Newport Beach, CA (US); Hieu Minh Luong, Westminster, CA (US); David Robert Landon, Huntington Beach, CA (US); Siddharth Vad, Irvine, CA (US); Ramon Aguilar, JR., Corona, CA (US); David M. Taylor, Lake Forest, CA (US); Jesse Robert Edwards, Silverado, CA (US); Brandon Sina Dehdashtian, Irvine, CA (US)

(21) Appl. No.: 18/658,829

(22) Filed: May 8, 2024

Related U.S. Application Data
(63) Continuation of application No. PCT/US2022/050636, filed on Nov. 21, 2022.
(60) Provisional application No. 63/281,812, filed on Nov. 22, 2021.

(51) Int. Cl. A61F 2/24

(52) U.S. Cl. CPC A61F 2/2418; A61F 2/243; A61F 2/2436; A61F 2220/0008

(57) ABSTRACT

Devices, systems, and methods relate to implants, such as prosthetic valves for replacing native heart valves. Delivery systems for delivering the implants to an implantation site are also disclosed herein. A prosthetic valve may include a valve body supporting a plurality of prosthetic valve leaflets and one or more adjustable anchors coupled to the valve body for anchoring the prosthetic valve to leaflets of a native heart valve, wherein at least one of the adjustable anchors is axially slidable relative to the valve body for conforming to the native anatomy of the native heart valve.

### CROSS-REFERENCE TO RELATED APPLICATIONS

This application is a continuation of International Application No. PCT/US2022/050636, filed Nov. 21, 2022, which designates the United States and was published in English by the International Bureau on May 25, 2023 as WO2023/091769, which claims the benefit of U.S. Provisional Application No. 63/281,812, filed Nov. 22, 2021, each of which is hereby incorporated by reference.

### BACKGROUND

### Field

Certain examples disclosed herein relate generally to implants for implantation within a body and delivery systems for an implant. In particular, the implants and delivery systems relate in some examples to replacement heart valves, such as replacement mitral or tricuspid heart valves.

### Background

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prostheses including but not limited to replacement heart valves that can be compacted for delivery and then controllably expanded for controlled placement has proven to be particularly challenging. An additional challenge relates to the ability of such prostheses to be secured relative to intralumenal tissue, e.g., tissue within any body lumen or cavity, in an atraumatic manner. The ability to control the deployment of the prosthesis at the desired location can also be challenging.

### SUMMARY

Examples of the present disclosure may be directed to implants and deployment of such implants, such as but not limited to a replacement heart valve. Examples of the present disclosure may also be directed to delivery systems, devices and/or methods of use to deliver and/or controllably deploy an implant, such as but not limited to a replacement heart valve, to a desired location within the body. In some examples, a replacement heart valve and methods for delivering a replacement heart valve to a native heart valve, such as a mitral or tricuspid valve, are provided.

The present disclosure includes, but is not limited to, the following examples. Examples may include a delivery system for an implant. The delivery system may comprise an elongate shaft having a distal end, an implant retention area for retaining the implant, a first bend portion configured to deflect the distal end in a first plane, a first extension portion positioned proximal of the first bend portion and extending along a first axis, a second bend portion positioned proximal of the first extension portion and configured to rotate in a first rotational direction in a second plane extending transverse to the first plane, a third bend portion positioned proximal of the second bend portion and configured to rotate in a second rotational direction that is opposite the first rotational direction in the second plane, and a second extension portion positioned proximal of the third bend portion and extending along a second axis. The delivery system may include a deflection mechanism configured to deflect the second bend portion in the first rotational direction in the second plane and deflect the third bend portion in the second rotational direction in the second plane to offset the first axis from the second axis with the first axis extending parallel with the second axis.

Examples may include a method comprising delivering a delivery apparatus for an implant into a portion of a patient's body. The delivery apparatus may include an elongate shaft having a distal end, an implant retention area retaining the implant, a first bend portion configured to deflect the distal end in a first plane, a first extension portion positioned proximal of the first bend portion and extending along a first axis, a second bend portion positioned proximal of the first extension portion and configured to rotate in a first rotational direction in a second plane extending transverse to the first plane, a third bend portion positioned proximal of the second bend portion and configured to rotate in a second rotational direction that is opposite the first rotational direction in the second plane, and a second extension portion positioned proximal of the third bend portion and extending along a second axis. The delivery apparatus may include a deflection mechanism configured to deflect the second bend portion in the first rotational direction in the second plane and deflect the third bend portion in the second rotational direction in the second plane to offset the first axis from the second axis with the first axis extending parallel with the second axis.

Examples may include a delivery system for an implant. The delivery system may comprise an elongate shaft having a retention body configured to retain the implant. The delivery system may comprise a diaphragm extending proximally and configured to be moved distally to allow the retention body to release from the implant.

Examples may include a method comprising delivering a delivery apparatus for an implant into a portion of a patient's body. The delivery apparatus may include an elongate shaft. The elongate shaft may include a retention body retaining the implant, and a diaphragm extending proximally and configured to be moved distally to allow the retention body to release from the implant.

Examples may include a delivery system for an implant. The delivery system may comprise one or more suction ports configured to apply suction to native heart valve leaflets of a native heart valve to draw the native heart valve leaflets radially inward. The delivery system may comprise an elongate shaft having an implant retention area and configured to at least partially deploy the implant from the implant retention area to the native heart valve with the one or more suction ports applying the suction to the native heart valve leaflets.

Examples may include a method comprising applying suction from one or more suction ports to native heart valve leaflets of a native heart valve to draw the native heart valve leaflets radially inward. The method may comprise at least partially deploying an implant from an elongate shaft having an implant retention area to the native heart valve with the one or more suction ports applying the suction to the native heart valve leaflets.

Examples may include a prosthetic valve configured to be deployed to a native valve. The prosthetic valve may include a plurality of prosthetic valve leaflets. The prosthetic valve may include a valve body supporting the plurality of prosthetic valve leaflets. The prosthetic valve may include a plurality of distal anchors each having a distal tip and configured to move from a crimped configuration to a deployed configuration, at least one of the distal tips in the crimped configuration being offset longitudinally from a position of another of the distal tips in the crimped configuration.

Examples may include a method comprising deploying a prosthetic valve to a native valve. The prosthetic valve may include a plurality of prosthetic valve leaflets, a valve body supporting the plurality of prosthetic valve leaflets, and a plurality of distal anchors each having a distal tip and configured to move from a crimped configuration to a deployed configuration, at least one of the distal tips in the crimped configuration being offset longitudinally from a position of another of the distal tips in the crimped configuration.

Examples may include a prosthetic valve configured to be deployed to a native valve. The prosthetic valve may include a plurality of prosthetic valve leaflets. The prosthetic valve may include a valve body supporting the plurality of prosthetic valve leaflets. The prosthetic valve may include a plurality of distal anchors each having a distal tip, at least one of the distal tips configured to have a greater diameter than another of the distal tips.

Examples may include a method comprising deploying a prosthetic valve to a native valve. The prosthetic valve may include a plurality of prosthetic valve leaflets, a valve body supporting the plurality of prosthetic valve leaflets, and a plurality of distal anchors each having a distal tip, at least one of the distal tips configured to have a greater diameter than another of the distal tips.

Examples may include a delivery system for an implant. The delivery system may comprise an elongate shaft including an implant retention area for retaining the implant. The delivery system may comprise one or more sutures configured to couple to at least one of a plurality of distal anchors of the implant and apply a compressive force to the at least one of the plurality of distal anchors radially inward.

Examples may include a method comprising utilizing a delivery system to deploy an implant to a portion of a patient's body. The delivery system may include an elongate shaft including an implant retention area for retaining the implant, and one or more sutures configured to couple to at least one of a plurality of distal anchors of the implant and apply a compressive force to the at least one of the plurality of distal anchors radially inward.

Examples may include a delivery system for an implant. The delivery system may comprise an elongate shaft including an implant retention area for retaining the implant. The delivery system may comprise one or more sutures configured to form a loop extending circumferentially about the implant and configured to apply a compressive force to the implant radially inward.

Examples may include a method comprising utilizing a delivery system to deploy an implant to a portion of a patient's body. The delivery system may include an elongate shaft including an implant retention area for retaining the implant, and one or more sutures forming a loop extending circumferentially about the implant and configured to apply a compressive force to the implant radially inward.

Examples may include a delivery system for an implant. The delivery system may comprise an elongate shaft including an implant retention area for retaining the implant and an inner shaft configured to pass through the implant. The delivery system may comprise one or more sutures including a first portion configured to couple to the implant and apply a compressive force to the implant radially inward and a second portion configured to couple to the inner shaft.

Examples may include a method comprising utilizing a delivery system to deploy an implant to a portion of a patient's body. The delivery system may include an elongate shaft including an implant retention area retaining the implant and an inner shaft configured to pass through the implant, and one or more sutures including a first portion configured to couple to the implant and apply a compressive force to the implant radially inward and a second portion configured to couple to the inner shaft.

Examples may include a prosthetic valve configured to be deployed to a native valve. The prosthetic valve may include a plurality of prosthetic valve leaflets. The prosthetic valve may include a valve body supporting the plurality of prosthetic valve leaflets. The prosthetic valve may include one or more anchors each configured to anchor the prosthetic valve to the native valve and each including a first arm and a second arm and configured to extend radially outward to a tip, the tip including a loop coupling the first arm to the second arm and the first arm configured to be moved relative to the second arm to vary a distance between the first arm and the second arm.

Examples may include a method comprising deploying a prosthetic valve to a native valve. The prosthetic valve may include a plurality of prosthetic valve leaflets, a valve body supporting the plurality of prosthetic valve leaflets, and one or more anchors each configured to anchor the prosthetic valve to the native valve and each including a first arm and a second arm and configured to extend radially outward to a tip, the tip including a loop coupling the first arm to the second arm and the first arm configured to be moved relative to the second arm to vary a distance between the first arm and the second arm.

Examples may include a prosthetic valve configured to be deployed to a native valve. The prosthetic valve may include a plurality of prosthetic valve leaflets. The prosthetic valve may include a valve body supporting the plurality of prosthetic valve leaflets. One or more anchors may each be configured to anchor the prosthetic valve to the native valve and each configured to slide relative to the valve body.

Examples may include a method comprising deploying a prosthetic valve to a native valve. The prosthetic valve may include a plurality of prosthetic valve leaflets, a valve body supporting the plurality of prosthetic valve leaflets, and one or more anchors each configured to anchor the prosthetic valve to the native valve and each configured to slide relative to the valve body.

Examples may include a delivery system for an implant. The delivery system may include an elongate shaft including an implant retention area for retaining the implant; and a control mechanism configured to control a deflection of at least one distal anchor of the implant independent of a deflection of at least one other distal anchor of the implant.

Examples may include a method comprising utilizing a delivery system to deploy an implant to a portion of a patient's body. The delivery system may include an elongate shaft including an implant retention area for retaining the implant, and a control mechanism configured to control a deflection of at least one distal anchor of the implant independent of a deflection of at least one other distal anchor of the implant.

Examples may include a prosthetic valve configured to be deployed to a native valve. The prosthetic valve may comprise a plurality of prosthetic valve leaflets; a valve body supporting the plurality of prosthetic valve leaflets; and one or more pacemaker leads configured to anchor the valve body in position at the native valve.

Examples may include a method comprising deploying a prosthetic valve to a native valve. The prosthetic valve may include a plurality of prosthetic valve leaflets; a valve body supporting the plurality of prosthetic valve leaflets; and one or more pacemaker leads configured to anchor the valve body in position at the native valve.

Examples may include a method comprising imaging a native heart valve; and manufacturing at least a portion of a prosthetic heart valve based on the imaging of the native heart valve.

Examples may include a prosthetic valve configured to be deployed to a native valve. The prosthetic valve may comprise a plurality of prosthetic valve leaflets; a valve body supporting the plurality of prosthetic valve leaflets; and wherein at least a portion of the prosthetic heart valve is manufactured based on imaging of the native heart valve.

### BRIEF DESCRIPTION OF THE DRAWINGS

FIG. 1 illustrates an example of a delivery system.

FIG. 2A illustrates a partial cross-sectional view of the distal end of the delivery system of FIG. 1 loaded with the implant of FIG. 3E.

FIG. 2B illustrates a partial cross-sectional view of the distal end of the delivery system of FIG. 1 without the implant of FIG. 3E.

FIG. 2C illustrates a partial cross-sectional view of the distal end of the delivery system of FIG. 1 with certain shaft assemblies translated along the rail assembly.

FIG. 3A illustrates a perspective view of an implant that may be delivered using delivery systems described herein.

FIG. 3B illustrates a bottom view of the implant shown in FIG. 3A.

FIG. 3C illustrates a cross sectional schematic view of the implant shown in FIG. 3A.

FIG. 3D illustrates a side view of an example of an aortic valve prosthesis that may be delivered using delivery systems described herein.

FIG. 3E illustrates a side view of an example of a valve prosthesis that may be delivered using the delivery systems described herein.

FIG. 4 illustrates a perspective view of the distal end of the delivery system of FIG. 1.

FIG. 5 illustrates components of the delivery system of FIG. 4 with the outer sheath assembly moved proximally and out of view.

FIG. 6A illustrates components of the delivery system of FIG. 5 with the mid shaft assembly moved proximally and out of view.

FIG. 6B illustrates a cross-section of the rail assembly.

FIG. 6C illustrates a cross-section of an example of the rail assembly.

FIG. 7 illustrates components of a delivery system.

FIG. 8 illustrates components of the delivery system of FIG. 7 with the inner assembly moved proximally and out of view.

FIG. 9 illustrates an example of a rail assembly.

FIG. 10 illustrates an example of a delivery system handle.

FIG. 11 illustrates a cross-section of the delivery system handle of FIG. 10.

FIG. 12A illustrates a side view of a distal end of an elongate shaft.

FIG. 12B illustrates a side view of the distal end of the elongate shaft deflected from the position shown in FIG. 12A.

FIG. 12C illustrates a top view of the distal end of the elongate shaft deflected from the position shown in FIG. 12A and within an atrium of the heart.

FIG. 13A illustrates a side view of a distal end of an elongate shaft.

FIG. 13B illustrates a side view of the distal end of the elongate shaft deflected from the position shown in FIG. 13A.

FIG. 13C illustrates a top view of the distal end of the elongate shaft deflected from the position shown in FIG. 13A and within an atrium of the heart.

FIG. 13D illustrates a top view of the distal end of the elongate shaft deflected from the position shown in FIG. 13C and within the atrium of the heart.

FIG. 13E illustrates a cross sectional view of a rail assembly.

FIG. 13F illustrates a cross sectional view of the rail assembly shown in FIG. 13E along line 13F-13F.

FIG. 13G illustrates a cross sectional view of the rail assembly shown in FIG. 13E along line 13G-13G.

FIG. 13H illustrates a cross sectional view of the rail assembly shown in FIG. 13E along line 13H-13H.

FIG. 14 illustrates a cross sectional view of a rail assembly.

FIG. 15A illustrates a perspective view of a rail assembly having cuts positioned on a tube of the rail assembly.

FIG. 15B illustrates a cross sectional view of a rail assembly having stoppers positioned thereon.

FIG. 16A illustrates a side view of a distal end of an elongate shaft.

FIG. 16B illustrate a top view of the distal end of the elongate shaft shown in FIG. 16A.

FIG. 17A illustrates a representation of an elongate shaft entering a right atrium of a patient's heart.

FIG. 17B illustrates a distal end of the elongate shaft shown in FIG. 17A being deflected from the position shown in FIG. 17A.

FIG. 17C illustrates a distal end of the elongate shaft shown in FIG. 17B being deflected from the position shown in FIG. 17B.

FIG. 18A illustrates a distal end of the elongate shaft aligned with a tricuspid valve.

FIG. 18B illustrates an inflation body inflated within an inferior vena cava.

FIG. 19 illustrates a representation of an elongate shaft entering a right atrium of a patient's heart from the superior vena cava.

FIG. 20A illustrates a perspective view of an implant being deployed from an elongate shaft.

FIG. 20B illustrates a perspective view of an implant being deployed from an elongate shaft.

FIG. 20C illustrates a perspective view of an implant being deployed from an elongate shaft.

FIG. 21 illustrates an implant in position within a tricuspid valve annulus.

FIG. 22A illustrates a perspective view of a diaphragm of an elongate shaft.

FIG. 22B illustrates a cross sectional view of the diaphragm shown in FIG. 22A.

FIG. 23A illustrates a perspective view of the diaphragm shown in FIG. 22A moved from the position shown in FIG. 22A.

FIG. 23B illustrates a cross sectional view of the diaphragm shown in FIG. 23A.

FIG. 24A illustrates a delivery system approaching a native valve.

FIG. 24B illustrates the delivery system shown in FIG. 24A applying suction to native heart valve leaflets.

FIG. 24C illustrates the delivery system shown in FIG. 24A at least partially deploying an implant to a native valve.

FIG. 24D illustrates the delivery system shown in FIG. 24A having deployed an implant to a native valve.

FIG. 24E illustrates a cross sectional view of a portion of a delivery system.

FIG. 25 illustrates a partial cross-sectional view of a distal end of a delivery system loaded with a prosthetic valve.

FIG. 26 illustrates a plan view of distal tips of anchors of a prosthetic valve.

FIG. 27 illustrates a plan view of distal tips of anchors of a prosthetic valve.

FIG. 28 illustrates a top cross sectional view of a prosthetic valve.

FIG. 29 illustrates a top cross sectional view of a prosthetic valve.

FIG. 30A illustrates a side view of a prosthetic valve deployed to a native valve, the prosthetic valve having inflatable tips.

FIG. 30B illustrates a side view of the prosthetic valve shown in FIG. 30A with tips inflated.

FIG. 31A illustrates a side view of a prosthetic valve deployed to a native valve, the prosthetic valve having inflatable tips.

FIG. 31B illustrates a perspective view of a tube coupled to an inflation conduit of a prosthetic valve.

FIG. 31C illustrates a side view of the prosthetic valve shown in FIG. 31A with tips inflated.

FIG. 32 illustrates shows a top cross sectional view of a prosthetic valve.

FIG. 33A illustrates a side cross sectional view of an implant positioned within an elongate shaft of a delivery system.

FIG. 33B illustrates a side cross sectional view of the implant shown in FIG. 33A being deployed to a native valve.

FIG. 33C illustrates a missed capture of a native valve leaflet by the implant shown in FIG. 33A.

FIG. 33D illustrates an anchor of an implant being compressed radially inward by a delivery system.

FIG. 34 illustrates a side cross sectional view of an implant deployed to a native valve.

FIG. 35A illustrates an implant being compressed radially inward by a delivery system.

FIG. 35B illustrates a top cross sectional view of an implant compressed radially inward by a delivery system.

FIG. 35C illustrates an implant being compressed radially inward by a delivery system.

FIG. 36 illustrates an implant being compressed radially inward by an inner shaft of a delivery system.

FIG. 37A illustrates an implant being compressed radially inward by an inner shaft of a delivery system.

FIG. 37B illustrates the implant shown in FIG. 37A being compressed radially inward by an inner shaft of a delivery system.

FIG. 38A illustrates an implant being compressed radially inward by an inner shaft of a delivery system.

FIG. 38B illustrates the implant shown in FIG. 38A being compressed radially inward by an inner shaft of a delivery system.

FIG. 39 illustrates an implant being compressed radially inward by an inner shaft of a delivery system.

FIG. 40A illustrates a side cross sectional view of an implant retained by a delivery system.

FIG. 40B illustrates a side cross sectional view of an implant shown in FIG. 40A being deployed to a native valve.

FIG. 40C illustrates a side cross sectional view of the implant shown in FIG. 40A with distal anchors released from the delivery system.

FIG. 40D illustrates a side cross sectional view of the implant shown in FIG. 40A deployed.

FIG. 41 illustrates a side cross sectional view of an implant deployed to a native valve.

FIG. 42 illustrates a side view of a tip of an implant.

FIG. 43 illustrates a side view of a tip of an implant.

FIG. 44 illustrates a top view of the tip of the implant shown in FIG. 43.

FIG. 45A illustrates a side cross sectional view of an implant retained by a delivery system.

FIG. 45B illustrates a side cross sectional view of an implant shown in FIG. 45A being deployed to a native valve.

FIG. 45C illustrates a side cross sectional view of the implant shown in FIG. 45A deployed.

FIG. 46A illustrates a side cross sectional view of an implant being deployed by a delivery system.

FIG. 46B illustrates a side cross sectional view of anchors of an implant being deployed.

FIG. 46C illustrates a side cross sectional view of an implant having a missed capture of a native valve leaflet.

FIG. 46D illustrates a side cross sectional view of an implant with an anchor being retracted.

FIG. 46E illustrates a side cross sectional view of an implant with an anchor retracted.

FIG. 46F illustrates a side cross sectional view of an implant deployed to a native valve.

FIG. 46G illustrates a top cross sectional view of the implant shown in FIG. 46F.

FIG. 46H illustrates a side view of an anchor shown in FIG. 46F.

FIG. 47 illustrates a cross sectional view of a lock of an anchor.

FIG. 48 illustrates a cross sectional view of a lock of an anchor.

FIG. 49 illustrates a cross sectional view of a lock of an anchor.

FIG. 50 illustrates a side cross sectional schematic view of a control mechanism.

FIG. 51A illustrates a side cross sectional schematic view of a control mechanism.

FIG. 51B illustrates a side cross sectional schematic view of a deployed implant.

FIG. 52A illustrates a side cross sectional schematic view of an implant within a capsule.

FIG. 52B illustrates a side cross sectional schematic view of an implant being deployed.

FIG. 52C illustrates a side cross sectional schematic view of an implant being deployed.

FIG. 53A illustrates a side cross sectional schematic view of an implant being deployed.

FIG. 53B illustrates a side cross sectional schematic view of an implant being deployed.

FIG. 53C illustrates a side cross sectional schematic view of an implant being deployed.

FIG. 54 illustrates a side cross sectional schematic view of a delivery system deploying an implant.

FIG. 55 illustrates a perspective cross sectional view of a portion of a control mechanism shown in FIG. 54.

FIG. 56 illustrates an assembly view of components of the control mechanism shown in FIG. 54.

FIG. 57 illustrates a side perspective view of a housing of a control mechanism shown in FIG. 54.

FIG. 58 illustrates a side cross sectional schematic view of the delivery system shown in FIG. 54 deploying an implant.

FIG. 59 illustrates a side cross sectional schematic view of the delivery system shown in FIG. 54 deploying an implant.

FIG. 60 illustrates a side cross sectional view of a portion of a control mechanism shown in FIG. 54.

FIG. 61 illustrates a side cross sectional schematic view of the delivery system shown in FIG. 54 adjusting distal anchors of an implant.

FIG. 62 illustrates a side cross sectional view of a portion of a control mechanism shown in FIG. 54.

FIG. 63 illustrates a side cross sectional schematic view of the delivery system shown in FIG. 54 adjusting a distal anchor of an implant.

FIG. 64 illustrates a side cross sectional schematic view of an implant deployed to a portion of a heart.

FIG. 65 illustrates a side cross sectional schematic view of an implant shown in FIG. 64 being deployed to a portion of a heart.

FIG. 66 illustrates a side cross sectional view of a lock.

FIG. 67 illustrates a side cross sectional schematic view of an implant shown in FIG. 64 deployed to a portion of a heart and coupled to a pacemaker.

FIG. 68 illustrates a side cross sectional schematic view of an implant deployed to a portion of a heart.

FIG. 69 illustrates a side cross sectional schematic view of an imaging procedure.

FIG. 70 illustrates a schematic view of a processing system.

FIG. 71 illustrates a side view of a fabrication assembly.

FIG. 72 illustrates a top cross sectional view of a mandrel.

FIG. 73 illustrates a side view of a fabrication assembly.

FIG. 74 illustrates a top cross sectional view of a valve body positioned upon a mandrel.

FIG. 75 illustrates a top cross sectional view of a valve body having an inner valve body and an outer valve body.

FIG. 76 illustrates a side view of a fabrication assembly.

FIG. 77 illustrates a perspective view of a prosthetic heart valve.

### DETAILED DESCRIPTION

The present specification and drawings provide aspects and features of the disclosure in the context of several examples of implants such as prosthetic valves, delivery systems, and methods that are configured for use in the vasculature of a patient, such as for replacement or repair of native heart valves in a patient. The prosthetic valves may comprise replacement heart valves or other forms of prosthetic valves. These examples may be discussed in connection with replacing specific valves such as the patient's aortic, tricuspid, mitral, or pulmonary valve. However, it is to be understood that the features and concepts discussed herein can be applied to devices other than implants for heart valves. For example, implants, the delivery systems, and methods can be applied to medical implants, for example other types of expandable prostheses, for use elsewhere in the body, such as within an artery, a vein, or other body cavities or locations. In addition, specific features of a prosthetic valve, delivery system, method, etc. should not be taken as limiting, and features of any one example discussed herein can be combined with features of other examples as desired and when appropriate. While certain of the examples described herein are described in connection with a transfemoral delivery approach, it should be understood that these examples can be used for other delivery approaches such as, for example, transapical, transatrial, or transjugular approaches. Moreover, it should be understood that certain of the features described in connection with certain examples can be incorporated with other examples, including those that are described in connection with different delivery approaches.

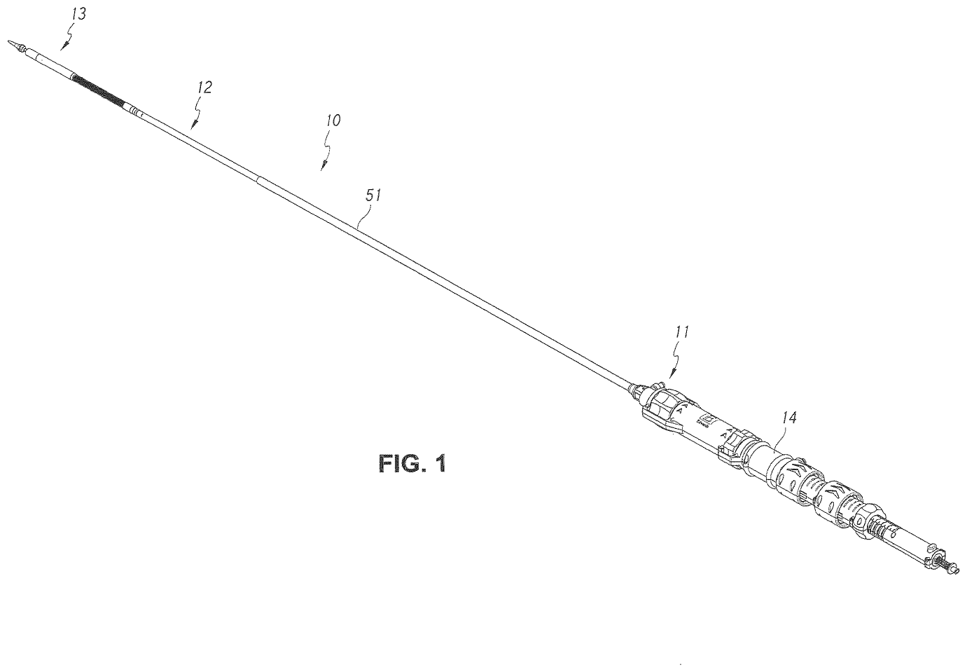

FIG. 1 illustrates an example of a delivery device, assembly, or system 10. The delivery system 10 can be used to deploy an implant, such as a prosthetic valve, within the body. In some examples, the delivery system 10 may use a dual plane deflection approach to properly deliver the implant. Implants such as prosthetic heart valves may be delivered to a patient's mitral valve or tricuspid heart valve annulus or other heart valve location, such as an aortic or pulmonary valve, in various manners, such as by open surgery, minimally-invasive surgery, and percutaneous or transcatheter delivery through the patient's vasculature. Example transfemoral approaches may be found in U.S. Pat. Pub. No. 2015/0238315, filed Feb. 20, 2015, the entirety of which is hereby incorporated by reference in its entirety. While the delivery system 10 is described in connection with a percutaneous delivery approach, and more specifically a transfemoral delivery approach, it should be understood that features of delivery system 10 can be applied to other delivery systems, including delivery systems for a transapical, transatrial, or transjugular delivery approach.

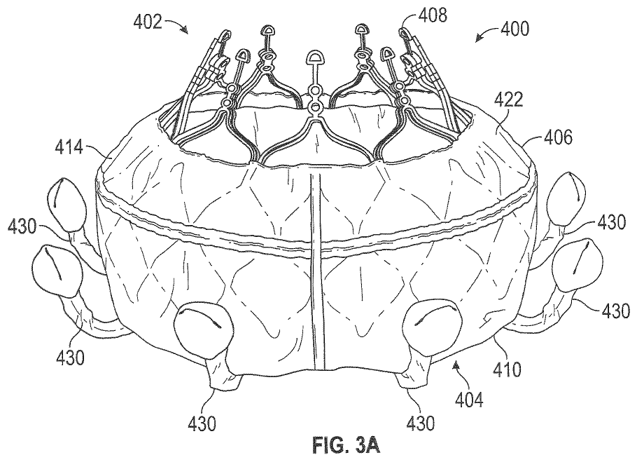

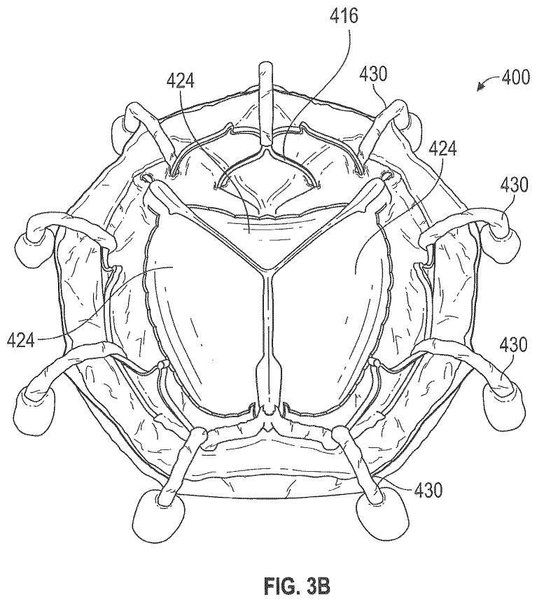

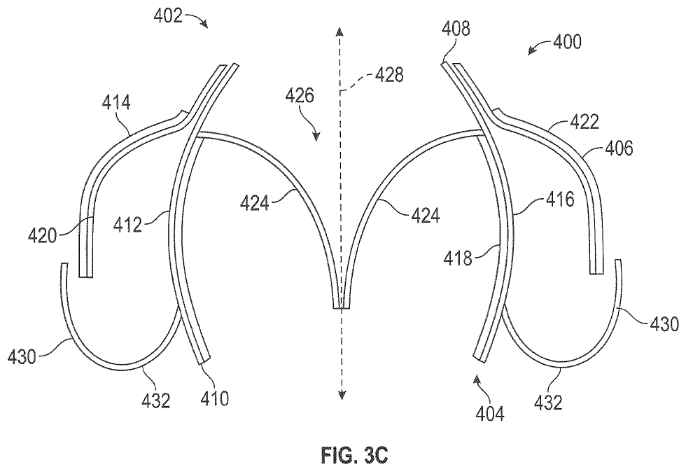

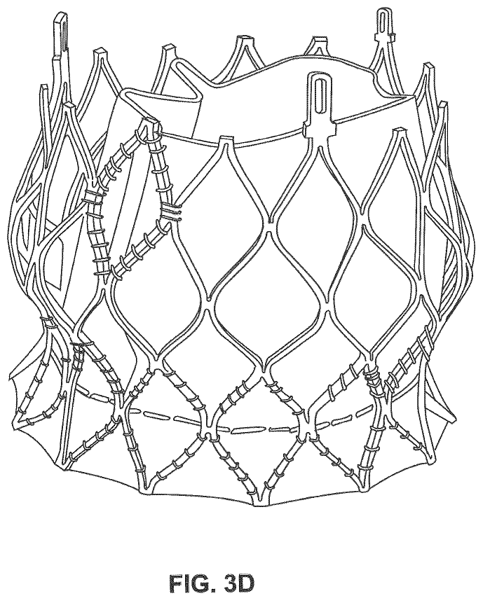

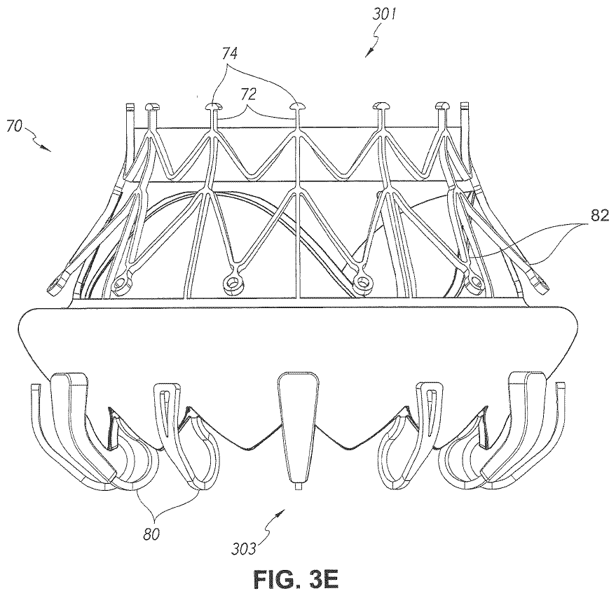

The delivery system 10 may be used to deploy a prosthesis, such as a replacement heart valve as described elsewhere in this specification, within the body. The delivery system 10 can receive and/or cover portions of the prosthesis such as a first end 301 and second end 303 of the prosthesis or implant 70 illustrated in FIG. 3E or the respective first ends and second ends of the implants shown in FIGS. 3A-3D. For example, the delivery system 10 may be used to deliver an expandable prosthesis or implant 70, where the implant 70 includes the first end 301 and the second end 303, and wherein the second end 303 is configured to be deployed or expanded before the first end 301.

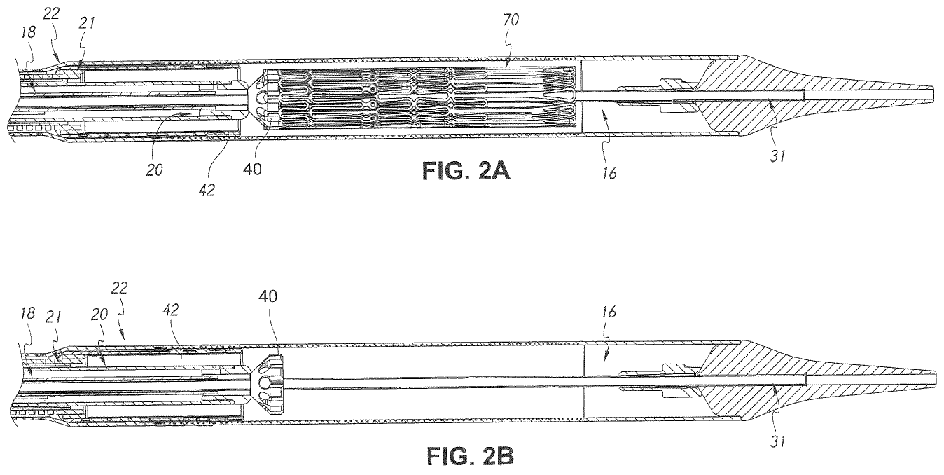

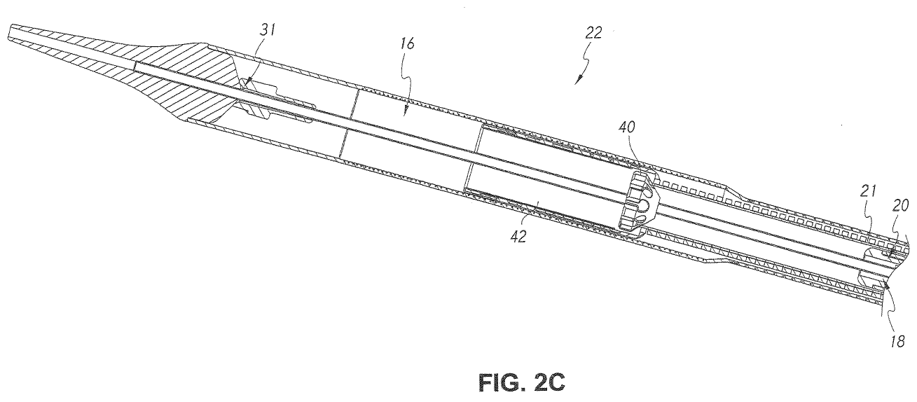

FIG. 2A further shows an example of an implant 70 (as marked in FIG. 3E) that can be inserted into the delivery system 10, specifically into the implant retention area 16. For case of understanding, in FIG. 2A, the implant is shown with only the bare metal frame illustrated. The implant 70 can take any number of different forms. For example, implants as shown in FIGS. 3A-3E may be utilized in examples, among other forms of implants. A particular example of a frame for an implant is shown in FIG. 3E, though it will be understood that other designs and frame configurations may also be used, including those disclosed in this application. An implant 70 may include one or more sets of anchors, such as distal (or ventricular) anchors 80 extending proximally when the prosthesis frame is in an expanded configuration and proximal (or atrial) anchors 82 extending distally when the prosthesis frame is in an expanded configuration. The prosthesis can further include struts 72 which may end in mushroom-shaped tabs 74 at the first end 301. Further discussion can be found in U.S. Publication No. 2015/0328000A1, published Nov. 19, 2015, hereby incorporated by reference in its entirety.

In some examples, the delivery system 10 can be used in conjunction with a replacement aortic valve, such as shown in FIG. 3D. In some examples the delivery system 10 can be modified to support and deliver the replacement aortic valve. However, the procedures and structures discussed below can similarly be used for a replacement tricuspid and replacement aortic valve.

FIGS. 3A-3C illustrate an example of an implant 400 that may be utilized according to examples herein. The implant 400 may include a proximal or first end 402 and a distal or second end 404. The implant 400 may include a valve body 406 having a proximal end 408 and a distal end 410.

FIG. 3C illustrates a schematic cross sectional view of the implant 400. In examples, the valve body 406 may include an inner body 412 and an outer body 414. The inner body 412 may include a valve frame 416, which may be referred to as an inner valve frame 416. The inner valve frame 416 may include a plurality of struts separated by openings. In examples, a scaling skirt 418 may be provided on the inner valve frame 416 that may reduce fluid flow through the scaling skirt 418.

The outer body 414 is positioned radially outward from the inner body 412 and may comprise a sealing body in examples. The outer body 414 may include a frame, which may be referred to as an outer frame 420 that may extend circumferentially around the inner valve frame 416. A scaling skirt 422 (as visible in FIG. 3A) may be coupled to the outer frame 420. The scaling skirt 422 may be configured to reduce fluid flow around the outer body 414 to reduce the possibility of fluid leakage around the outer body 414 (e.g., paravalvular leakage). In examples, the outer body 414 may be configured to be flexible to allow the outer body 414 to conform to a shape of an implantation site, such as a valve annulus.

The valve body 406 may support a plurality of prosthetic valve leaflets 424 (marked in FIG. 3B). The plurality of prosthetic valve leaflets 424 may be positioned within a flow channel 426 of the implant 400 that fluid may flow through. The prosthetic valve leaflets 424 may be configured to open and close to mimic the operation of a native valve in examples. The valve body 406 may surround a central axis 428 that may pass through the flow channel 426.

One or more anchors 430 may be coupled to the valve body 406. In the example shown in FIGS. 3A-3C, the anchors 430 may comprise distal anchors and may extend from the distal end 410 of the valve body 406. The anchors 430 may be configured to extend radially outward from the valve body 406 and may have a hooked shape. In examples, the distal anchors may be configured to hook over a distal tip of a leaflet of a native valve, with the distal tip of each of the plurality of distal anchors being positioned radially outward of the leaflet. Other configurations of examples may be utilized as desired. In examples, a proximal portion 432 of the distal anchors 430 may be coupled to a distal portion of the valve body 406, among other configurations.

The implant 400 is shown in FIGS. 3A-3C in a deployed configuration, yet may be in a crimped configuration. The implant 400 in a crimped configuration may be compressed for insertion and delivery to an implantation site. For example, the implant 400 may be compressed radially inward, and the anchors 430 may be elongated and extend along the central axis 428. Such a configuration is shown in FIG. 2A with regard to the implant 70 of FIG. 3E. The implant shown in FIG. 3D may similarly be held in a crimped configuration and expanded to a deployed configuration in examples. In examples, the implant may comprise a self-expanding implant configured to move from the crimped configuration to the deployed configuration.

Additional details and example designs for a prosthesis are described in U.S. Pat. Nos. 8,403,983, 8,414,644, 8,652,203 and U.S. Patent Publication Nos. 2011/0313515, 2012/0215303, 2014/0277390, 2014/0277422, 2014/0277427, 2018/0021129, and 2018/0055629, the entirety of these patents and publications are hereby incorporated by reference and made a part of this specification. Further details and examples of a replacement heart valve or prosthesis and its method of implantation are described in U.S. Publication Nos. 2015/0328000 and 2016/0317301 the entirety of each of which is hereby incorporated by reference and made a part of this specification.

The delivery system 10 can be relatively flexible. In some examples, the delivery system 10 is particularly suitable for delivering an implant in the form of a prosthetic valve to an implantation site such as a mitral valve location through a transseptal approach (e.g., between the right atrium and left atrium via a transseptal puncture). The delivery system 10, however, may be suitable for delivering a replacement heart valve to a tricuspid valve location, among other locations.

As shown in FIG. 1, the delivery system 10 can include a shaft assembly or elongate shaft 12 comprising a proximal end 11 and a distal end 13, wherein a handle 14 is coupled to the proximal end of the elongate shaft 12. The elongate shaft 12 can be used to hold the implant 70 for advancement of the same through the vasculature to a treatment location. The delivery system 10 can further comprise a relatively rigid live-on (or integrated) sheath 51 surrounding the elongate shaft 12 that can prevent unwanted motion of the elongate shaft 12. The live-on sheath 51 can be attached at a proximal end of elongate shaft 12 proximal to the handle 14, for example at a sheath hub. The elongate shaft 12 can include an implant retention area 16 (shown in FIGS. 2A-2B with FIG. 2A showing the implant 70 of FIG. 3E and FIG. 2B with the implant 70 removed) at its distal end that can be used for this purpose. In some examples, the elongate shaft 12 can hold an expandable implant in a compressed state at implant retention area 16 for advancement of the implant within the body. The elongate shaft 12 may then be used to allow controlled expansion of the implant at the treatment location. In some examples, the elongate shaft 12 may be used to allow for sequential controlled expansion of the implant as discussed in detail below. The implant retention area 16 is shown in FIGS. 2A-2B at the distal end of the delivery system 10, but may also be at other locations. In some examples, the implant may be rotated in the implant retention area 16, such as through the rotation of the inner shaft assembly 18 discussed herein.

As shown in cross-sectional view of FIGS. 2A-2B, the distal end of the delivery system 10 can include one or more subassemblies such as an outer sheath assembly 22, a mid shaft assembly 21, a rail assembly 20, an inner shaft assembly 18, and a nose cone assembly 31 as will be described in more detail below. In some examples, the delivery system 10 may not have all of the assemblies disclosed herein. For example, in some examples a full mid shaft assembly may not be incorporated into the delivery system 10. In some examples, the assemblies disclosed below may be in a different radial order than is discussed.

In particular, examples of the disclosed delivery system 10 can utilize a steerable rail in the rail assembly 20 for steering the distal end of the delivery system 10, allowing the implant to be properly located in a patient's body. As discussed in detail below, the steerable rail can be, for example, a rail shaft that extends through the delivery system 10 from the handle 14 generally to the distal end. In some examples, the steerable rail has a distal end that ends proximal to the implant retention area 16. A user can manipulate the bending of the distal end of the rail, thereby bending the rail in a particular direction. In preferred examples, the rail has more than one bend along its length, thereby providing multiple directions of bending. As the rail is bent, it presses against the other assemblies to bend them as well, and thus the other assemblies of the delivery system 10 can be configured to steer along with the rail as a cooperating single unit, thus providing for full steerability of the distal end of the delivery system.

Once the rail is steered into a particular location in a patient's body, an implant such as the implant 70 shown in FIG. 3E can be advanced along or relative to the rail through the movement of the other sheaths/shafts relative to the rail and released into the body. For example, the rail can be bent into a desired position within the body, such as to direct the implant towards a native valve such as a native mitral valve or a native tricuspid valve. The other assemblies (e.g., the outer sheath assembly 22, the mid shaft assembly 21, the inner shaft assembly 18, and the nose cone assembly 31) can passively follow the bends of the rail. Further, the other assemblies (e.g., the outer sheath assembly 22, the mid shaft assembly 21, the inner shaft assembly 18, and the nose cone assembly 31) can be advanced together (e.g., relatively together, sequentially with one actuator, simultaneously, almost simultaneously, at the same time, closely at the same time) relative to the rail while maintaining the implant in the compressed position without releasing or expanding the implant (e.g., within the implant retention area 16). The other assemblies (e.g., the outer sheath assembly 22, the mid shaft assembly 21, the inner shaft assembly 18, and the nose cone assembly 31) can be advanced distally or proximally together relative to the rail. In some examples, only the outer sheath assembly 22, mid shaft assembly 21, and inner shaft assembly 18 are advanced together over the rail. Thus, the nose cone assembly 31 may remain in the same position. The assemblies can be individually, sequentially, or simultaneously, translated relative to the inner shaft assembly 18 in order to release the implant from the implant retention area 16.

FIG. 2C illustrates the sheath assemblies, specifically the outer sheath assembly 22, the mid shaft assembly 21, the inner shaft assembly 18, and the nose cone assembly 31 having translated distally together along the rail assembly 20, further details on the assemblies are below. In some examples, the outer sheath assembly 22, the mid shaft assembly 21, the inner shaft assembly 18, and the nose cone assembly 31 translate together (e.g., relatively together, sequentially with one actuator, simultaneously, almost simultaneously, at the same time, closely at the same time). This distal translation can occur while the implant remains in a compressed configuration within the implant retention area 16.

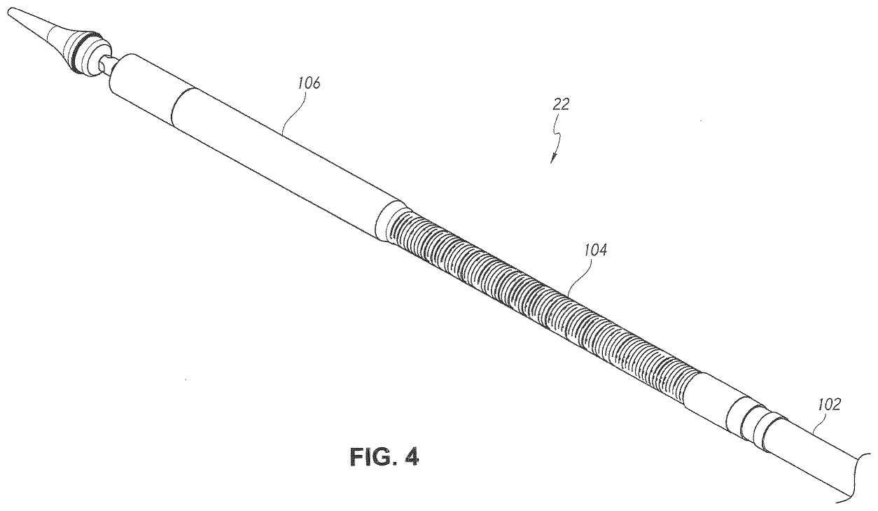

As shown in FIGS. 2A-2C and as further shown in FIGS. 4-8, starting with the outermost assembly, the delivery system can include an outer sheath assembly 22 forming a radially outer covering, or sheath, to surround an implant retention area 16 and prevent the implant from radially expanding. Specifically, the outer sheath assembly 22 can prevent the distal end of the implant from radially expanding. Moving radially inward, the mid shaft assembly 21 can be composed of a mid shaft hypotube 43 with its distal end attached to an outer retention member or outer retention ring 42 for radially retaining a portion of the prosthesis in a compacted configuration, such as a proximal end of the implant. The mid shaft assembly 21 can be located within a lumen of the outer sheath assembly 22. Moving further inwards, the rail assembly 20 can be configured for steerability, as mentioned above and further described below. The rail assembly 20 can be located within a lumen of the mid shaft assembly 21. Moving further inwards, the inner shaft assembly 18 can be composed of an inner shaft with its distal end attached to inner retention member or inner retention ring 40 (such as a PEEK ring) for axially retaining the implant, for example the proximal end of the implant. The inner shaft assembly 18 can be located within a lumen of the rail assembly 20. Further, the most radially-inward assembly is the nose cone assembly 31 which includes the nose cone shaft 27 having its distal end connected to the nose cone 28. The nose cone 28 can have a tapered tip. The nose cone assembly 31 is preferably located within a lumen of the inner shaft assembly 18. The nose cone assembly 31 can include a lumen for a guide wire to pass therethrough.

The elongate shaft 12, and more specifically the nose cone assembly 31, inner shaft assembly 18, rail assembly 20, mid shaft assembly 21, and outer sheath assembly 22, can be collectively configured to deliver an implant positioned within the implant retention area 16 (shown in FIG. 2A) to a treatment location. One or more of the subassemblies can then be moved to allow the implant to be released at the treatment location. For example, one or more of the subassemblies may be movable with respect to one or more of the other subassemblies. The handle 14 can include various control mechanisms that can be used to control the movement of the various subassemblies as will also be described in more detail below. In this way, the implant can be controllably loaded onto the delivery system 10 and then later deployed within the body. Further, the handle 14 can provide steering to the rail assembly 20, providing for bending/flexing/steering of the distal end of the delivery system 10.

As will be discussed below, the inner retention member 40, the outer retention ring 42, and the outer sheath assembly 22 can cooperate to hold the implant in a compacted configuration. The inner retention member 40 is shown engaging struts 72 at the proximal end 301 of the implant 70 in FIG. 2A. For example, slots located between radially extending teeth on the inner retention member 40 can receive and engage the struts 72 which may end in mushroom-shaped tabs 74 on the proximal end of the implant 70. The mid shaft assembly 21 can be positioned over the inner retention member 40 so that the first end 301 of the implant 70 is trapped between the inner retention member 40 and the outer retention ring 42, thereby securely attaching it to the delivery system 10 between the mid shaft assembly 21 and the inner retention member 40. The outer sheath assembly 22 can be positioned to cover the second end 303 of the implant 70.

The outer retention member 42 may be attached to a distal end of the mid shaft hypotube 43 which can in turn be attached to a proximal tube 44 at a proximal end, which in turn can be attached at a proximal end to the handle 14. The outer retention member 42 can provide further stability to the implant when in the compressed position. The outer retention member 42 can be positioned over the inner retention member 40 so that the proximal end of the implant 70 is trapped therebetween, securely attaching it to the delivery system 10. The outer retention member 42 can encircle a portion of an implant such as a first end of the implant shown in FIG. 3A-3D or the implant 70 shown in FIG. 3E, in particular the first end 301, thus preventing the implant 70 from expanding. Further, the mid shaft assembly 21 can be translated proximally with respect to the inner shaft assembly 18 into the outer sheath assembly 22, thus exposing a first end 301 of an implant 70 held within the outer retention member 42. In this way the outer retention member 42 can be used to help secure an implant to or release it from the delivery system 10. The outer retention member 42 can have a cylindrical or elongate tubular shape, and may be referred to as an outer retention ring, though the particular shape is not limiting.

As shown in FIG. 2A, the distal anchors of an implant can be located in a delivered configuration where the distal anchors (such as distal anchors 80 shown in FIG. 3E) point generally distally (as illustrated, axially away from the main body of the prosthesis frame and away from the handle of the delivery system). The distal anchors can be restrained in this delivered configuration by the outer sheath assembly 22. Accordingly, when the outer sheath 22 is withdrawn proximally, the distal anchors can flip positions (e.g., bend approximately 180 degrees) to a deployed configuration (e.g., pointing generally proximally) as shown in FIGS. 3A-3C and 3E. FIG. 2A also shows the proximal anchors 82 of the example of FIG. 3E extending distally in their delivered configuration within the outer sheath assembly 22. In other examples, the distal anchors 80 can be held to point generally proximally in the delivered configuration and compressed against the body of the prosthesis frame.

The delivery system 10 may be provided to users with an implant preinstalled. In other examples, an implant can be loaded onto the delivery system shortly before use, such as by a physician or nurse.

FIGS. 4-8 illustrate further views of the delivery system 10 with different assemblies translated proximally and described in detail.

Starting with the outermost assembly shown in FIG. 4, the outer sheath assembly 22 can include an outer proximal shaft 102 directly attached to the handle 14 at its proximal end and an outer hypotube 104 attached at its distal end. A capsule 106 can then be attached generally at the distal end of the outer hypotube 104. In some examples, the capsule 106 can be 28 French or less in size. These components of the outer sheath assembly 22 can form a lumen for the other subassemblies to pass through.

A capsule 106 can be located at a distal end of the outer proximal shaft 102. The capsule 106 can be a tube formed of a plastic or metal material. In some examples, the capsule 106 is formed of ePTFE or PTFE. In some examples, this capsule 106 is relatively thick to prevent tearing and to help maintain a self-expanding implant in a compacted configuration. In some examples the material of the capsule 106 is the same material as the coating on the outer hypotube 104. As shown, the capsule 106 can have a diameter larger than the outer hypotube 104, though in some examples the capsule 106 may have a similar diameter as the hypotube 104. In some examples, the capsule 106 may include a larger diameter distal portion and a smaller diameter proximal portion. In some examples, there may be a step or a taper between the two portions. The capsule 106 can be configured to retain an implant in a compressed position within the capsule 106. Further construction details of the capsule 106 are discussed below.

The outer sheath assembly 22 is configured to be individually slidable with respect to the other assemblies. Further, the outer sheath assembly 22 can slide distally and proximally relative to the rail assembly 20 together with the mid shaft assembly 21, inner shaft assembly 18, and nose cone assembly 31.

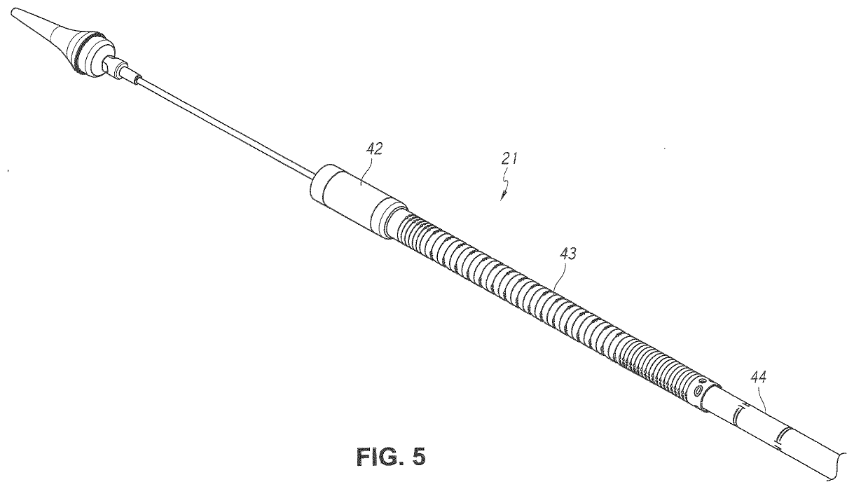

Moving radially inwardly, the next assembly is the mid shaft assembly 21. FIG. 5 shows a similar view as FIG. 4, but with the outer sheath assembly 22 removed, thereby exposing the mid shaft assembly 21.

The mid shaft assembly 21 can include a mid shaft hypotube 43 generally attached at its proximal end to a mid shaft proximal tube 44, which in turn can be attached at its proximal end to the handle 14, and an outer retention ring 42 located at the distal end of the mid shaft hypotube 43. Thus, the outer retention ring 42 can be attached generally at the distal end of the mid shaft hypotube 43. These components of the mid shaft assembly 21 can form a lumen for other subassemblies to pass through.

The outer retention ring 42 can be configured as a prosthesis retention mechanism that can be used to engage with an implant, as discussed with respect to FIG. 2A. For example, the outer retention ring 42 may be a ring or covering that is configured to radially cover struts on the implant. The outer retention ring 42 can also be considered to be part of the implant retention area 16, and may be at the proximal end of the implant retention area 16. With struts or other parts of an implant engaged with the inner retention member 40, discussed below the outer retention ring 42 can cover both the implant and the inner retention member 40 to secure the implant on the delivery system 10. Thus, the implant can be sandwiched between the inner retention member 40 of the inner shaft assembly 18 and the outer retention ring 42 of the mid shaft assembly 21.

The mid shaft assembly 21 is disposed so as to be individually slidable with respect to the other assemblies. Further, mid shaft assembly 21 can slide distally and proximally relative to the rail assembly 20 together with the outer sheath assembly 22, the inner shaft assembly 18, and nose cone assembly 31.

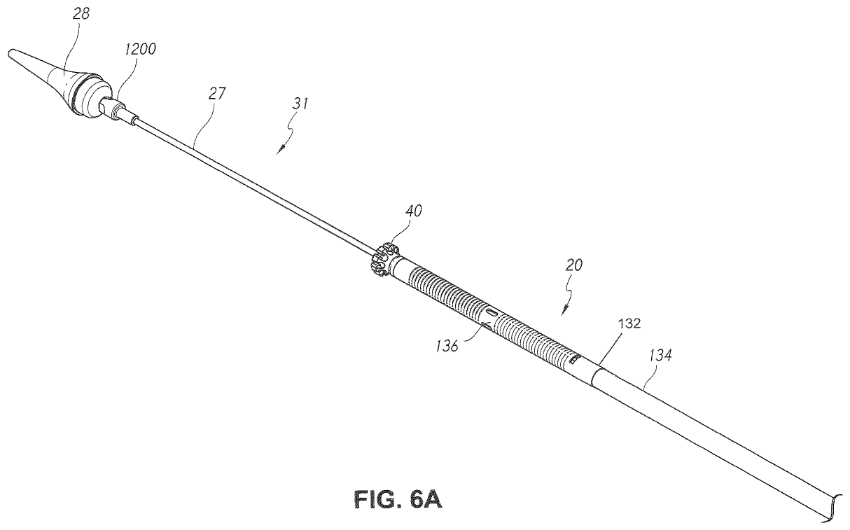

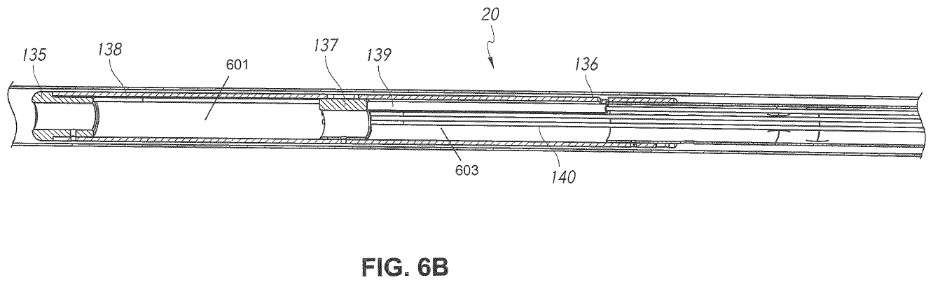

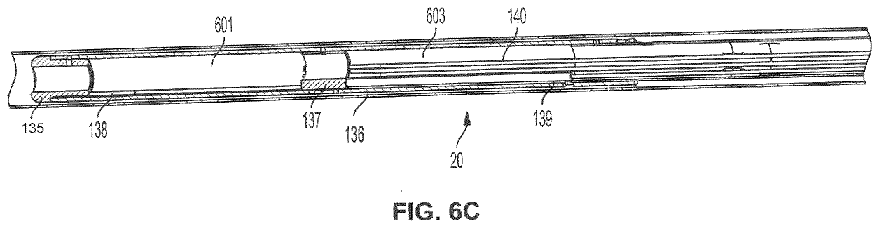

Next, radially inwardly of the mid shaft assembly 21 is the rail assembly 20. FIG. 6A shows approximately the same view as FIG. 5, but with the mid shaft assembly 21 removed, thereby exposing the rail assembly 20. FIG. 6B further shows a cross-section of the rail assembly 20 to view the pull tethers in the form of pull wires. The rail assembly 20 can include a rail shaft 132 (or rail) generally attached at its proximal end to the handle 14. The rail shaft 132 can be made up of a rail proximal shaft 134 directly attached to the handle at a proximal end and a rail hypotube 136 attached to the distal end of the rail proximal shaft 134. The rail shaft 132 may include a proximal rail shaft portion 603 and a distal rail shaft portion 601. The rail hypotube 136 can further include an atraumatic rail tip at its distal end. Further, the distal end of the rail hypotube 136 can abut a proximal end of the inner retention member 40, as shown in FIG. 6A. In some examples, the distal end of the rail hypotube 136 can be spaced away from the inner retention member 40. These components of the rail shaft assembly 20 can form a lumen for the other subassemblies to pass through.

As shown in FIG. 6B, attached to an inner surface of the rail hypotube 136 are one or more pull tethers in the form of pull wires which can be used apply forces to the rail hypotube 136 and steer the rail assembly 20. The pull wires can extend distally from the knobs in the handle 14, discussed below, to the rail hypotube 136. In some examples, pull wires can be attached at different longitudinal locations on the rail hypotube 136, thus providing for multiple bending locations in the rail hypotube 136, allowing for multidimensional steering.

In some examples, a distal pull wire 138 can extend to a distal section of the rail hypotube 136 and two proximal pull wires 140 can extend to a proximal section of the rail hypotube 136, however, other numbers of pull wires can be used, and the particular amount of pull wires is not limiting. For example, two pull wires can extend to a distal location and a single pull wire can extend to a proximal location. In some examples, ring-like structures attached inside the rail hypotube 136, known as pull wire connectors, can be used as attachment locations for the pull wires, such as proximal ring 137 and distal ring 135. In some examples, the rail assembly 20 can include a distal pull wire connector 135 and a proximal pull wire connector 137. In some examples, the pull wires can directly connect to an inner surface of the rail hypotube 136.

The distal pull wire 138 can be connected (either on its own or through a connector 135) generally at the distal end of the rail hypotube 136. The proximal pull wires 140 can connect (cither on its own or through a connector 137) at a location approximately one quarter, one third, or one half of the length up the rail hypotube 136 from the proximal end. In some examples, the distal pull wire 138 can pass through a small diameter pull wire lumen 139 (e.g., tube, hypotube, cylinder) attached on the inside of the rail hypotube 136. This can prevent the wires 138 from pulling on the rail hypotube 136 at a location proximal to the distal connection. Further, the lumen 139 can act as compression coils to strengthen the proximal portion of the rail hypotube 136 and prevent unwanted bending. Thus, in some examples the lumen 139 is only located on the proximal half of the rail hypotube 136. In some examples, multiple lumens 139, such as spaced longitudinally apart or adjacent, can be used per distal wire 138. In some examples, a single lumen 139 is used per distal wire 138. In some examples, the lumen 139 can extend into the distal half of the rail hypotube 136. In some examples, the lumen 139 is attached on an outer surface of the rail hypotube 136. In some examples, the lumen 139 is not used.

For the pair of proximal pull wires 140, the wires can be spaced approximately 180° from one another to allow for steering in both directions. Similarly, if a pair of distal pull wires 138 is used, the wires can be spaced approximately 180° from one another to allow for steering in both directions. In some examples, the pair of distal pull wires 138 and the pair of proximal pull wires 140 can be spaced approximately 90° from each other. In some examples, the pair of distal pull wires 138 and the pair of proximal pull wires 140 can be spaced approximately 0° from each other. However, other locations for the pull wires can be used as well, and the particular location of the pull wires is not limiting. In some examples, the distal pull wire 138 can pass through a lumen 139 attached within the lumen of the rail hypotube 136. This can prevent an axial force on the distal pull wire 138 from creating a bend in a proximal section of the rail hypotube 136.

FIG. 6C illustrates an example in which the position of the proximal pull tethers or pull wires 140 has been moved 180° from the position shown in FIG. 6B. The position of the proximal pull wires 140 shown in FIG. 6C may allow the proximal portion of the rail hypotube 136 to bend in an opposite direction than the direction possible in FIG. 6B. For example, in the example of FIG. 6B, when the distal portion of the rail hypotube 136 is deflected in a downward direction by the pull of the distal pull wires 138, the proximal portion of the rail hypotube 136 may be deflected leftward relative to the downward direction (viewing from the proximal end of the rail hypotube 136 toward the distal end of the rail hypotube 136). In the example of FIG. 6C, however, when the distal portion of the rail hypotube 136 is deflected in a downward direction by the pull of the distal pull wires 138, the proximal portion of the rail hypotube 136 may be deflected rightward relative to the downward direction (viewing from the proximal end of the rail hypotube 136 toward the distal end of the rail hypotube 136). Such a variation may allow the proximal portion of the rail hypotube 136, and accordingly the elongate shaft 12 to deflect in an opposite direction than possible in the example shown in FIG. 6B. The thickness of cuts on the rail shaft 132 may also be varied to allow for the opposite direction of deflection.

The rail assembly 20 is disposed so as to be slidable over the inner shaft assembly 18 and the nose cone assembly 31. In some examples, the outer sheath assembly 22, the mid shaft assembly 21, the inner shaft assembly 18, and the nose cone assembly 31 can be configured to slide together along or relative to the rail assembly 20, such as proximally and distally with or without any bending of the rail assembly 20. In some examples, the outer sheath assembly 22, the mid shaft assembly 21, the inner shaft assembly 18, and the nose cone assembly 31 can be configured to retain the implant 70 in a compressed position when they are simultaneously slid along or relative to the rail assembly 20.

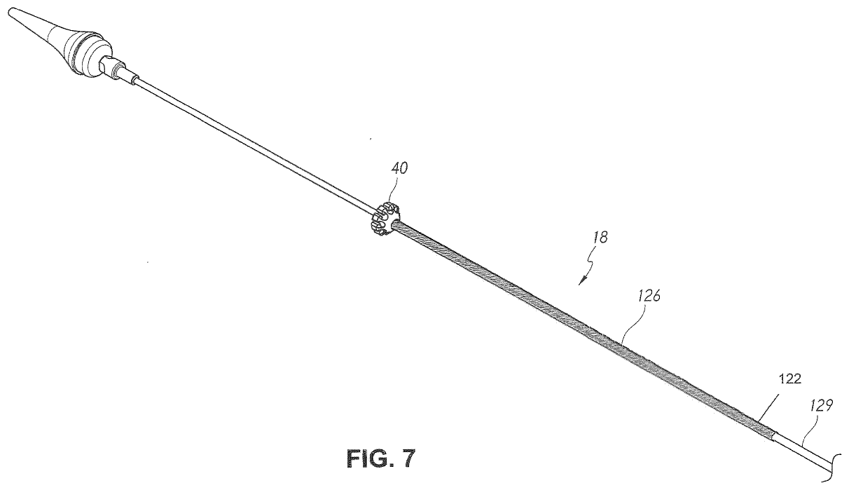

Moving radially inwards, the next assembly is the inner shaft assembly 18. FIG. 7 shows approximately the same view as FIG. 6A, but with the rail assembly 20 removed, thereby exposing the inner shaft assembly 18.

The inner shaft assembly 18 can include an inner shaft 122 generally attached at its proximal end to the handle 14, and an inner retention ring 40 located at the distal end of the inner shaft 122. The inner shaft 122 itself can be made up of an inner proximal shaft 129 directly attached to the handle 14 at a proximal end and a distal section 126 attached to the distal end of the inner proximal shaft 129. Thus, the inner retention ring 40 can be attached generally at the distal end of the distal section 126. These components of the inner shaft assembly 18 can form a lumen for the other subassemblies to pass through.

The inner retention member 40 can be configured as a prosthesis retention mechanism that can be used to engage with an implant, as discussed with respect to FIG. 2A. For example, the inner retention member 40 may be a ring and can include a plurality of slots configured to engage with struts 72 on an implant 70 as shown in FIG. 3E for example. The inner retention member 40 can also be considered to be part of the implant retention area 16, and may be at the proximal end of the implant retention area 16. With struts or other parts of an implant engaged with the inner retention member 40, the outer retention ring 42 can cover both the implant and the inner retention member 40 to secure the implant on the delivery system 10. Thus, an implant can be sandwiched between the inner retention member 40 of the inner shaft assembly 18 and the outer retention ring 42 of the mid shaft assembly 21.

The inner shaft assembly 18 is disposed so as to be individually slidable with respect to the other assemblies. Further, the inner shaft assembly 18 can slide distally and proximally relative to the rail assembly 20 together with the outer sheath assembly 22, mid shaft assembly 21, and nose cone assembly 31.

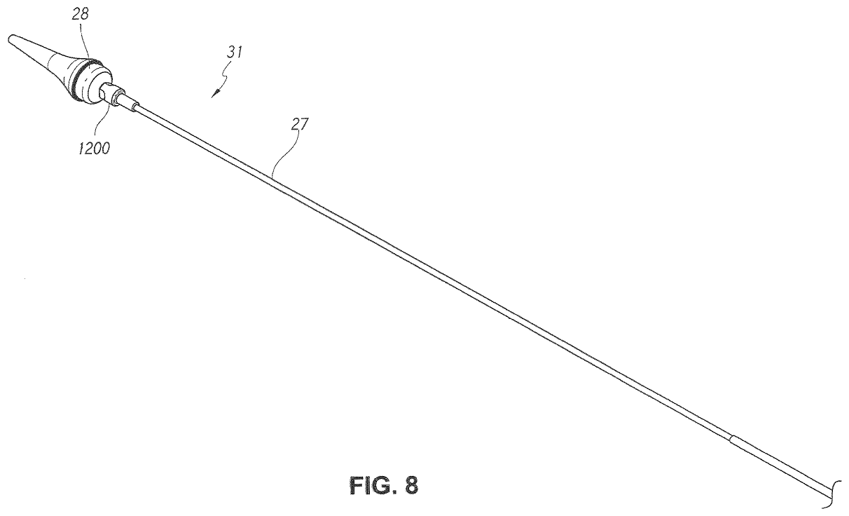

Moving further inwardly from the inner shaft assembly 18 is the nose cone assembly 31 also seen in FIG. 8. This may be a nose cone shaft 27, and in some examples, may have a nose cone 28 on its distal end. The nose cone 28 can be made of polyurethane for atraumatic entry and to minimize injury to venous vasculature. The nose cone 28 can also be radiopaque to provide for visibility under fluoroscopy.

The nose cone shaft 27 may include a lumen sized and configured to slidably accommodate a guide wire so that the delivery system 10 can be advanced over the guide wire through the vasculature. However, examples of the system 10 discussed herein may not use a guide wire and thus the nose cone shaft 27 can be solid. The nose cone shaft 27 may be connected from the nose cone 28 to the handle, or may be formed of different segments such as the other assemblies. Further, the nose cone shaft 27 can be formed of different materials, such as plastic or metal, similar to those described in detail above.

In some examples, the nose cone shaft 27 includes a guide wire shield 1200 located on a portion of the nose cone shaft 27.

The nose cone assembly 31 is disposed so as to be individually slidable with respect to the other assemblies. Further, the nose cone assembly 31 can slide distally and proximally relative to the rail assembly 20 together with the outer sheath assembly 22, mid shaft assembly 21, and inner shaft assembly 18.

In some examples, one or more spacer sleeves (not shown) can be used between different assemblies of the delivery system 10. For example, a spacer sleeve can be located concentrically between the mid shaft assembly and the rail assembly 20, generally between the mid 43 and rail hypotubes 136. In some examples, the spacer sleeve can be generally embedded in the hypotube 43 of the mid shaft assembly 21, such as on an inner surface of the mid shaft assembly 21. In some examples, a spacer sleeve can be located concentrically between the rail assembly 20 and the inner shaft assembly 18, generally within the rail hypotube 136. In some examples, a spacer sleeve can be used between the outer sheath assembly 22 and the mid shaft assembly 21. In some examples, a spacer sleeve can be used between the inner shaft assembly 18 and the nose cone assembly 31. In some examples, 4, 3, 2, or 1 of the above-mentioned spacer sleeves can be used. The spacer sleeves can be used in any of the above positions.

As discussed above, the outer sheath assembly 22, the mid shaft assembly 21, the inner shaft assembly 18, and the rail assembly 20 can contain an outer hypotube 104, a mid shaft hypotube, a distal section 126, and a rail hypotube 136, respectively. Each of these hypotubes/sections/shafts can be laser cut to include a number of slots, thereby creating a bending pathway for the delivery system to follow.

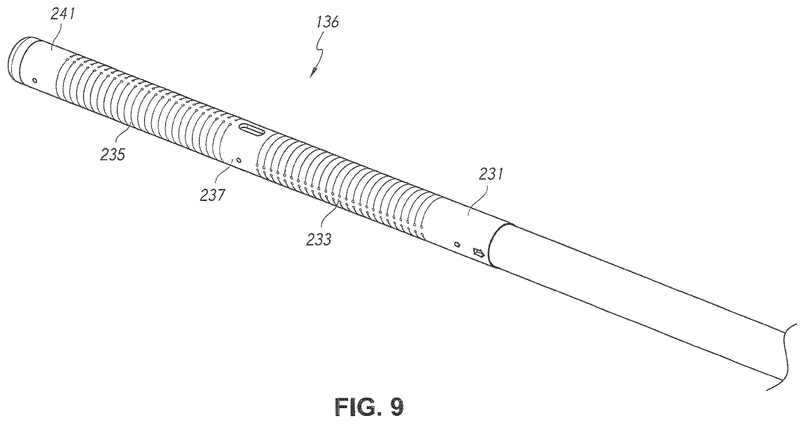

For example, FIG. 9 shows an example of the rail hypotube 136. The rail hypotube 136 can also contain a number of circumferential slots. The rail hypotube 136 can generally be broken into a number of different sections. At the most proximal end is an uncut (or unslotted) hypotube section 231. Moving distally, the next section is the proximal slotted hypotube section 233. This section includes a number of circumferential slots cut into the rail hypotube 136. Generally, two slots are cut around each circumferential location forming almost half of the circumference. Accordingly, two backbones are formed between the slots extending up the length of the hypotube 136. This is the section that can be guided by the proximal pull wires 140. Moving further distally is the location 237 where the proximal pull wires 140 connect, and thus slots can be avoided. This section may be just distal of the proximally slotted section.

Distally following the proximal pull wire connection area is the distal slotted hypotube section 235. This section is similar to the proximal slotted hypotube section 233, but has significantly more slots cut out in an equivalent length. Thus, the distally slotted hypotube section 235 provides casier bending than the proximally slotted hypotube section 233. The proximal and distal slotted hypotube sections 233, 235 may comprise bend portions of the rail shaft. In some examples, the proximal slotted section 233 can be configured to experience a bend of approximately 90 degrees with a half inch radius whereas the distal slotted section 235 can bend at approximately 180 degrees within a half inch. Further, as shown in FIG. 9, the spines of the distally slotted hypotube section 235 are offset from the spines of the proximally slotted hypotube section 233. Accordingly, the two sections will achieve different bend patterns, allowing for three-dimensional steering of the rail assembly 20. In some examples, the spines can be offset 30, 45, or 90 degrees, though the particular offset is not limiting. In some examples, the proximally slotted hypotube section 233 can include compression coils. This allows for the proximally slotted hypotube section 233 to retain rigidity for specific bending of the distally slotted hypotube section 235.

At the distalmost end of the distal slotted hypotube section 235 is the distal pull tether connection area 241 which is again a non-slotted section of the rail hypotube 136.

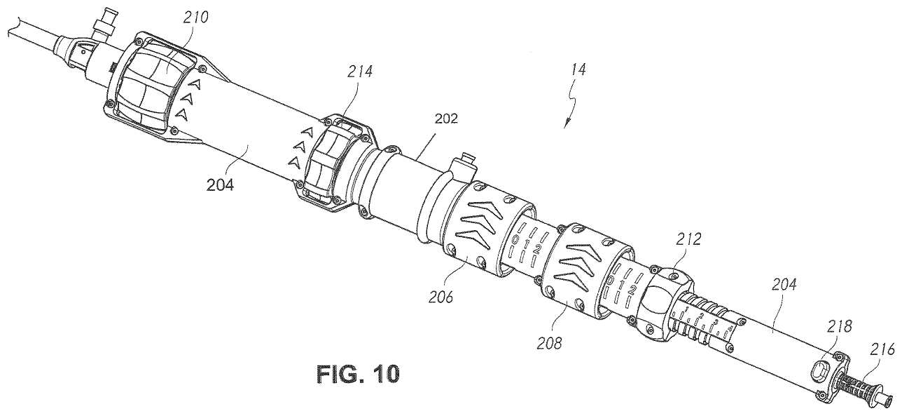

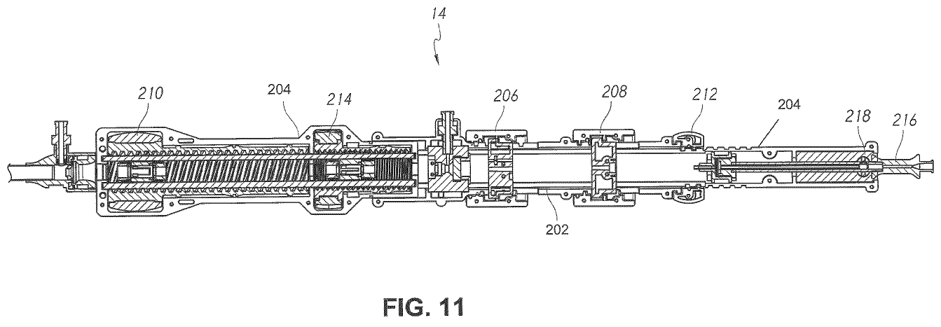

The handle 14 is located at the proximal end of the delivery system 10. An example of a handle 14 is shown in FIG. 10. A cross-section of the handle 14 is shown in FIG. 11. The handle 14 can include a number of actuators, such as rotatable knobs, that can manipulate different components of the delivery system 10. The operation of the handle 14 is described with reference to delivery of a replacement valve prosthesis or implant such as shown in FIGS. 3A-3E, though the handle 14 and delivery system 10 can be used to deliver other devices as well.

The handle 14 is generally composed of two housings, a rail housing 202 and a delivery housing 204, the rail housing 202 being circumferentially disposed around the delivery housing 204. The inner surface of the rail housing 202 can include a screwable section configured to mate with an outer surface of the delivery housing 204. Thus, the delivery housing 204 is configured to slide (e.g., screw) within the rail housing 202, as detailed below. The rail housing 202 generally surrounds about one half the length of the delivery housing 204, and thus the delivery housing 204 extends both proximally and distally outside of the rail housing 202.

The rail housing 202 can contain two rotatable knobs, a distal pull wire knob 206 and a proximal pull wire knob 208. However, the number of rotatable knobs on the rail housing 202 can vary depending on the number of pull wires used. Rotation of the distal pull wire knob 206 can provide a proximal force, thereby providing axial tension on the distal pull wires 138 and causing the distal slotted section of the rail hypotube 136 to bend. The distal pull wire knob 206 can be rotated in either direction, allowing for bending in either direction, which can control anterior-posterior angles. Rotation of the proximal pull wire knob 208 can provide a proximal force, and thus axial tension, on the proximal pull wires 140, thereby causing the proximal slotted section 233 of the rail hypotube 136 to bend, which can control the medial-lateral angle. The proximal pull wire knob 208 can be rotated in either direction, allowing for bending in either direction. Thus, when both knobs are actuated, there can be two bends in the rail hypotube 136, thereby allowing for three-dimensional steering of the rail shaft 132, and thus the distal end of the delivery system 10. Further, the proximal end of the rail shaft 132 is connected on an internal surface of the rail housing 202.

The bending of the rail shaft 132 can be used to position the system, in particular the distal end, at the desired patient location, such as at the native tricuspid valve. In some examples, rotation of the pull wire knobs 206/208 can help steer the distal end of the delivery system 10 to a desired position proximal a valve to be treated, for example a tricuspid or mitral valve.

Moving to the delivery housing 204, the proximal ends of the inner shaft assembly 18, outer sheath assembly 22, mid shaft assembly 21, and nose cone shaft assembly 31 can be connected to an inner surface of the delivery housing 204 of the handle 14. Thus, they can move axially relative to the rail assembly 20 and rail housing 202.

A rotatable outer sheath knob 210 can be located on the distal end of the delivery housing 204, being distal to the rail housing 202. Rotation of the outer sheath knob 210 will pull the outer sheath assembly 22 in an axial direction proximally, thus pulling the capsule 106 away from the implant covered by the capsule and releasing the distal end of the implant. Thus the outer sheath assembly 22 is individually translated with respect to the other shafts in the delivery system 10. A distal end of an implant can be released first, while a proximal end of the implant can remain radially compressed between the inner retention member 40 and the outer retention member 42.

A rotatable mid shaft knob 214 can be located on the delivery housing 204, in some examples proximal to the rotatable outer sheath knob 210, being distal to the rail housing 202. Rotation of the mid shaft knob 214 will pull the mid shaft assembly 21 in an axial direction proximally, thus pulling the outer retention ring 42 away from the implant and uncovering the inner retention member 40 and the proximal end of the implant, thereby releasing the implant. Thus, the mid shaft assembly 21 is individually translated with respect to the other shafts in the delivery system 10.

Located on the proximal end of the delivery housing 204, and thus proximal to the rail housing 202, can be a rotatable depth knob 212. As the depth knob 212 is rotated, the entirety of the delivery housing 204 moves distally or proximally with respect to the rail housing 202 which will remain in the same location. Thus, at the distal end of the delivery system 10, the inner shaft assembly 18, outer sheath assembly 22, mid shaft assembly 21, and nose cone shaft assembly 31 together (e.g., simultaneously) move proximally or distally with respect to the rail assembly 20 while the implant 70 remains in the compressed configuration. In some examples, actuation of the depth knob 212 can sequentially move the inner shaft assembly 18, outer sheath assembly 22, mid shaft assembly 21, and nose cone shaft assembly 31 relative to the rail assembly 20. In some examples, actuation of the depth knob 212 can together move the inner shaft assembly 18, outer sheath assembly 22, and mid shaft assembly 21 relative to the rail assembly 20. Accordingly, the rail shaft 132 can be aligned at a particular direction, and the other assemblies can move distally or proximally with respect to the rail shaft 132 for final positioning while not releasing the implant. The components can be advanced approximately 1, 2, 3, 5, 6, 7, 8, 9, or 10 cm along the rail shaft 132. The components can be advanced more than approximately 1, 2, 3, 5, 6, 7, 8, 9, or 10 cm along the rail shaft 132. An example of this is shown in FIG. 2C. The capsule 106 and outer retention ring 42 can then be individually withdrawn with respect to the inner shaft assembly 18 as discussed above, in some examples sequentially, releasing the implant. The assemblies other than the rail assembly 20 can then be withdrawn back over the rail shaft 132 by rotating the depth knob 212 in the opposite direction.

The handle 14 can further include a mechanism (knob, button, handle) 216 for moving the nose cone shaft 27, and thus the nose cone 28. For example, a knob 216 can be a portion of the nose cone assembly 31 that extends from a proximal end of the handle 14. Thus, a user can pull or push on the knob 216 to translate the nose cone shaft 27 distally or proximally individually with respect to the other shafts. This can be advantageous for proximally translating the nose conc 28 into the outer sheath assembly 22/capsule 106, thus facilitating withdraw of the delivery system 10 from the patient.

In some examples, the handle 14 can provide a lock 218, such as a spring lock, for preventing translation of the nose cone shaft 27 by the knob 216 discussed above. In some examples, the lock 218 can be always active, and thus the nose cone shaft 27 will not move without a user disengaging the lock 218. The lock can be, for example, a spring lock that is always engaged until a button 218 on the handle 14 is pressed, thereby releasing the spring lock and allowing the nose cone shaft 27 to translate proximally/distally. In some examples, the spring lock 218 allows one-way motion, either proximal or distal motion, of the nose cone shaft 27 but prevents motion in the opposite direction.

The handle 14 can further include a communicative flush port for flushing out different lumens of the delivery system 10. In some examples, a single flush port on the handle 14 can provide fluid connection to multiple assemblies. In some examples, the flush port can provide fluid connection to the outer sheath assembly 22. In some examples, the flush port can provide fluid connection to the outer sheath assembly 22 and the mid shaft assembly 21. In some examples, the flush port can provide fluid connection to the outer sheath assembly 22, the mid shaft assembly 21, and the rail assembly 20. In some examples, the flush port can provide fluid connection to the outer sheath assembly 22, the mid shaft assembly 21, the rail assembly 20, and the inner shaft assembly 18. Thus, in some examples, the rail shaft 132, the outer retention ring 42, and the capsule 106 can all be flushed by a single flush port.

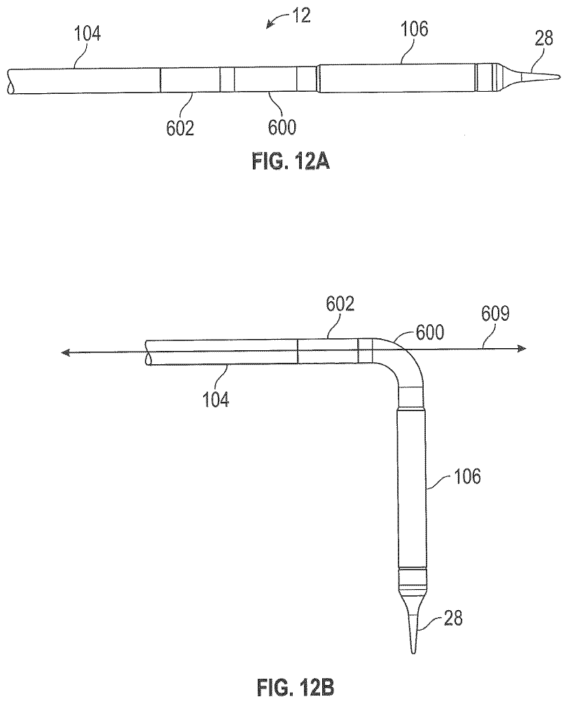

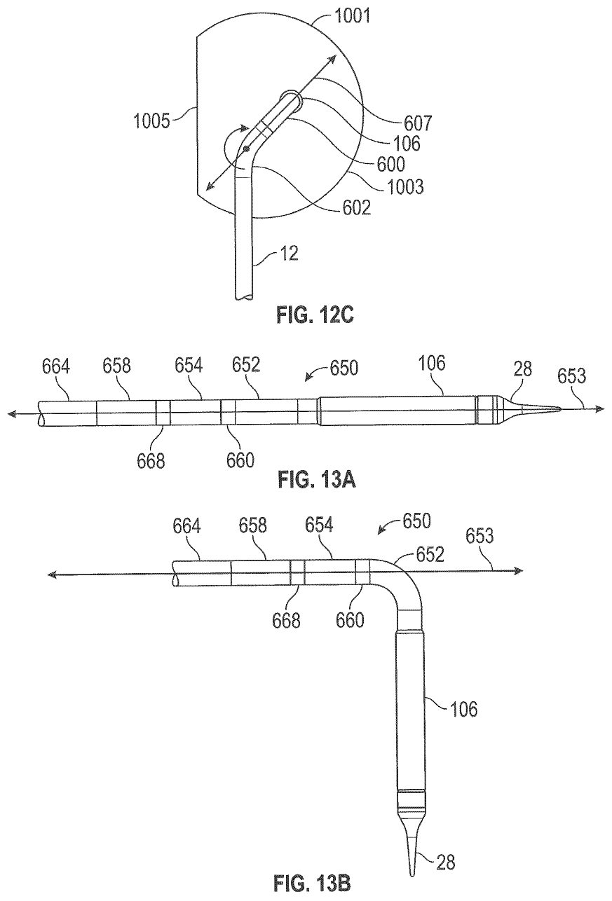

FIG. 12A illustrates a side view of a distal portion of the elongate shaft 12 with the elongate shaft 12 in a straightened configuration. The capsule 106 is shown positioned between the outer hypotube 104 and the nose cone 28.

The elongate shaft 12 may include one or more bend portions, which may allow the elongate shaft 12 to bend at the bend portions. In the example shown in FIG. 12A, for example, the elongate shaft 12 includes two bend portions 600, 602. The bend portion 600 may correspond to the distal rail portion 601 shown in FIGS. 6B and 6C, and the bend portion 602 may correspond to the proximal rail portion 603 shown in FIGS. 6B and 6C. As such, the bend portions 600, 602 may be configured to bend the elongate shaft 12 in planes that are perpendicular from each other, with the bend portion 600 able to bend in what may be called a vertical plane, and the bend portion 602 able to bend in what may accordingly be called a horizontal plane. The bend portions 600, 602 may be configured to bend to orient the capsule 106 in the desired position for deployment of the implant contained therein.

The capsule 106 (and the implant retention area 16 contained therein) may be configured to slide relative to the bend portions 600, 602 in the manners disclosed herein. For example, the outer sheath assembly 22, mid shaft assembly 21, inner shaft assembly 18, and nose cone assembly 31 may be configured to slide relative to the bend portions 600, 602 (as part of the rail assembly 20) to vary a distance or depth of the capsule 106 from the rail assembly 20. The outer sheath assembly 22 may be configured to slide relative to the rail assembly 20 to vary a distance of the implant retention area from a native valve to which the implant is deployed, such as a patient's mitral or tricuspid valve.

Referring to FIG. 12B, the bend portion 600, which is positioned proximal of the capsule 106, and is positioned between the capsule 106 and the bend portion 602, is shown to deflect the distal end of the elongate shaft 12 to a direction (which may be referred to as a downward direction as shown in FIG. 12B). The bend portion 600 deflects the distal end of the elongate shaft 12 in a plane 607 marked in FIG. 12C (which may be referred to as a vertical plane). The bend portion 600 accordingly has varied the orientation of the capsule 106, the distal end of the elongate shaft 12, and the implant retention area 16 positioned within the capsule 106.

FIG. 12C illustrates a top view of the elongate shaft 12 shown in FIGS. 12A and 12B, with the bend portion 602 bent. In FIG. 12C, the elongate shaft 12 is shown positioned within a right atrium of a heart, and positioned above a tricuspid heart valve including anterior 1001, posterior 1003, and septal 1005 portions. In FIG. 12C, the bend portion 602, which is positioned proximal the bend portion 600 is shown to deflect the distal end of the elongate shaft 12 to a direction (which may be referred to as a rightward direction as shown in FIG. 12C). The bend portion 602 deflects the distal end of the elongate shaft 12 in a plane 609 marked in FIG. 12B (which may be referred to as a horizontal plane). The bend portion 602 accordingly has varied the orientation of the capsule 106, the distal end of the elongate shaft 12, and the implant retention area 16 positioned within the capsule 106.

The bend portion 602 may deflect the bend portion 600 and the capsule 106 in a plane that is perpendicular to the plane that the bend portion 600 may deflect the capsule 106. The orthogonal planes of deflection may allow for three-dimensional steering of the capsule 106.

The bend portion 602 as shown in FIG. 12C may be configured to deflect the distal end of the elongate shaft 12 to a rightward direction. Such direction of deflection may be provided by the configuration of pull tethers shown in FIG. 6C.

Control of the elongate shaft 12 may be complex due to the visualization of the elongate shaft 12 during a procedure. The bend portion 600, for example, may deflect in a vertical plane, and the bend portion 602 may deflect in a horizontal plane, which may involve understanding of the geometry of the elongate shaft 12 and the movements of the bend portions 600, 602 to determine how to locate the capsule in a desired position relative to the native valve. For example, to move the capsule 106 to a specific position relative to the anterior 1001, posterior 1003, and septal 1005 portions, the user must determine a movement of both the bend portion 602 and the bend portion 600, with the bend portion 602 providing an arcuate movement that affects the position of the bend portion 600. Control of such arcuate movement may be technical and involved to produce. The deflection of the bend portion 602 affects a direction of deflection of the bend portion 600. Improvements in the case of use of an elongate shaft of a delivery system may be desired.

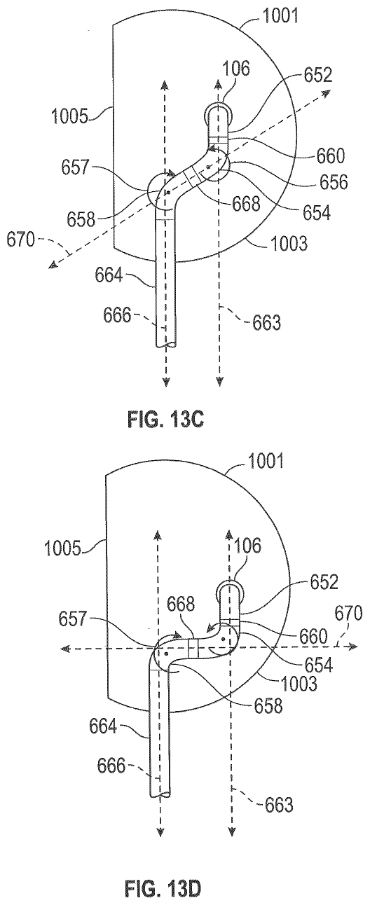

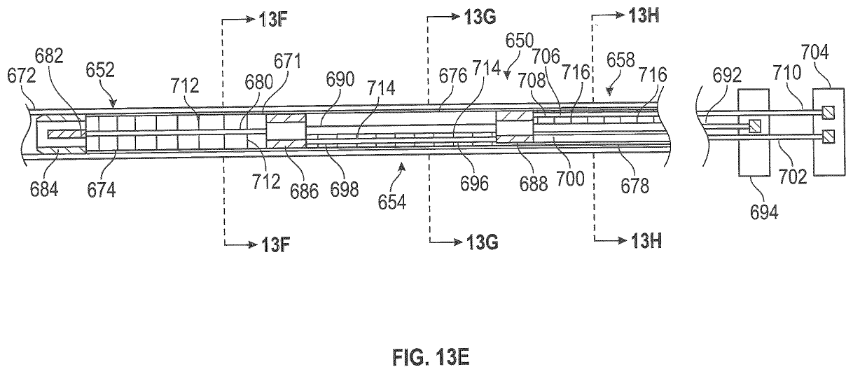

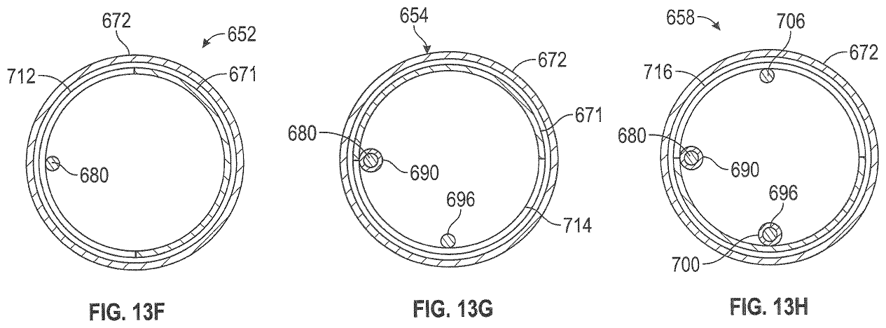

FIG. 13A illustrates an example of an elongate shaft 650 that may be utilized with a delivery system. The delivery system may be configured similarly as examples of delivery systems disclosed herein, or may have another configuration in examples as desired.

The elongate shaft 650 may include a distal end, which may include an implant retention area that may be configured similarly as the implant retention area 16 shown in FIGS. 2A and 2B for example. The implant retention area may include a capsule 106 that may cover the implant retention area and may retract for deployment of the implant retained by the capsule 106. The distal end of the elongate shaft 650 may include a nose cone 28 that may be positioned distal of the capsule 106 and the implant retention area.

The elongate shaft 650 may include a distal or first bend portion 652 that may be configured similarly as the bend portion 600 described in regard to FIGS. 12A-12C. The distal bend portion 652, for example, may be configured to deflect the distal end in a plane such as the plane 607 marked in FIG. 12C (which may be referred to as a vertical plane). The distal bend portion 652 may be configured to deflect the distal end in a downward direction (as shown in FIG. 13B) and may return to align the distal end to be coaxial with a proximal portion of the elongate shaft 650 (as shown in FIG. 13A). The implant retention area within the capsule 106 may be positioned distal of the distal bend portion 652.

The elongate shaft 650 may include an intermediate or second bend portion 654 that may be positioned proximal of the distal bend portion 652. The intermediate bend portion 654 may be configured to rotate in a rotational direction in a plane 653 that may extend transverse to the plane 607. The plane 653 may be a horizontal plane in examples and may extend perpendicular to the plane 607 that the distal bend portion 652 rotates in. FIG. 13C, for example, illustrates the rotation of the intermediate bend portion 654 in the rotational direction (marked by arrow 656). The intermediate bend portion 654 may be configured to deflect the distal bend portion 652, and the distal end of the elongate shaft 650.

The elongate shaft 650 may include a proximal or third bend portion 658 that may be positioned proximal of the intermediate bend portion 654. The proximal bend portion 658 may be configured to rotate in a rotational direction in the plane 653 that the intermediate bend portion 654 rotates in. The proximal bend portion 658 may be configured to rotate in a rotational direction (marked by arrow 657) that may be opposite the rotational direction of the intermediate bend portion 654 in the plane 653. For example, in FIG. 13C, the intermediate bend portion 654 is shown to rotate in a counterclockwise rotational direction and the proximal bend portion 658 is shown to rotate in a clockwise rotational direction. In examples, such a configuration may be reversed (with the intermediate bend portion 654 rotating in a clockwise rotational direction and the proximal bend portion 658 rotating in a counterclockwise rotational direction). The proximal bend portion 658 may be configured to deflect the intermediate bend portion 654 and the distal bend portion 652, and the distal end of the elongate shaft 650.

The elongate shaft 650 may include a distal or first extension portion 660 that may be positioned proximal of the distal bend portion 652 and distal of the intermediate bend portion 654. The distal extension portion 660 may comprise a connecting portion that joins the distal and intermediate bend portions 652, 654 and may have a relatively short length or a long length as desired. The distal extension portion 660, for example, may comprise a divider between the direction of rotation of the distal bend portion 652 relative to the intermediate bend portion 654. The distal extension portion 660 may extend along an axis 663 (as marked in FIG. 13C, for example).

The elongate shaft 650 may include a proximal or second extension portion 664 that may be positioned proximal of the proximal bend portion 658. The proximal extension portion 664 may connect the proximal bend portion 658 to the handle 14, and may have a relatively short length or a long length as desired. The proximal extension portion 664 may extend along an axis 666 (as marked in FIG. 13C, for example).

The elongate shaft 650 may include an intermediate or third extension portion 668 that may be positioned between the proximal bend portion 658 and the intermediate bend portion 654. The intermediate extension portion 668 may be positioned distal of the proximal bend portion 658 and proximal of the intermediate bend portion 654. The intermediate extension portion 668 may comprise a connecting portion that joins the intermediate and proximal bend portions 654, 658 and may have a relatively short length or a long length as desired. The intermediate extension portion 668, for example, may comprise a divider between the direction of rotation of the intermediate bend portion 654 relative to the proximal bend portion 658. The intermediate extension portion 668 may extend along an axis 670 (as marked in FIG. 13C, for example) and may be configured to extend transverse to the axis 663 of the distal extension portion 660 and the axis 666 of the proximal extension portion 664.

The bend portions 652, 654, 658 and the extension portions 660, 664, 668 may be configured to be coaxial with each other, as shown in FIG. 13A, for example. The axis 663 shown in FIG. 13C that the distal extension portion 660 extends along, for example, may be coaxial with the axis 666 that the proximal extension portion 664 extends along. The bend portions 652, 654, 658 may each be straightened.

A deflection mechanism may be utilized to deflect the bend portions 652, 654, 658 to orient a portion of the elongate shaft 650 as desired. A deflection mechanism may be configured to deflect the distal bend portion 652 to deflect the distal end of the elongate shaft 650 in a plane (such as the plane 607 shown in FIG. 12C for example). FIG. 13B illustrates the distal bend portion 652 having deflected the distal end of the elongate shaft 650 in a downward direction. The distal bend portion 652 deflects the distal end of the elongate shaft 650 to extend transverse to the axis 663 of the distal extension portion 660. The distal bend portion 652 may deflect the distal end of the elongate shaft 650 to extend perpendicular to the axis 663 of the distal extension portion 660.

The deflection mechanism may be configured to deflect the intermediate bend portion 654 in the rotational direction (indicated by arrow 656) in the plane 653 and deflect the proximal bend portion 658 in the rotational direction (indicated by arrow 657) in the plane 653 to offset the axis 663 from the axis 666 with the axis 663 extending parallel with the axis 666. FIG. 13C, for example, illustrates such a deflection of the intermediate and proximal bend portions 654, 658. In FIG. 13C, the distal end and capsule 106 are deflected in a downward direction in the plane (such as the plane 607 shown in FIG. 12C). The intermediate bend portion 654 rotates in the rotational direction indicated by arrow 656. The proximal bend portion 658 rotates in the rotational direction indicated by arrow 657. The rotation of the intermediate and proximal bend portions 654, 658 results in the offset of the axis 663 from the axis 666. The intermediate extension portion 668 and the axis 670 extend transverse to the axes 663, 666. The intermediate extension portion 668 extends diagonal relative to the proximal extension portion 664 and the distal extension portion 660.

The offset of the axis 663 from the axis 666 results in a lateral displacement of the position of the distal end of the elongate shaft 650 and the capsule 106 and a retraction of the distal end of the elongate shaft 650 and the capsule 106. Rotation of the capsule 106 and the distal end of the elongate shaft 650 is reduced or eliminated upon the rotation of the intermediate and proximal bend portions 654, 658. The resulting lateral movement of the distal end of the elongate shaft 650 and the capsule 106 may be relative to the proximal extension portion 664 that may extend to the handle 14 of the delivery system. The parallel configuration of the axes 666, 663 may reduce or eliminate the rotation of the capsule and the distal end of the elongate shaft 650.

The intermediate bend portion 654 may be configured to be deflected by the deflection mechanism in the rotational direction (indicated by arrow 656) a same amount as the deflection of the proximal bend portion 658 in the rotational direction (indicated by arrow 657). For example, a same degree or angle of rotation of each of the intermediate and proximal bend portions 654, 658, although in opposite directions, may allow the axis 663 to remain parallel with the axis 666 upon the lateral displacement of the distal end of the elongate shaft 650 and the capsule 106. In examples, the deflection of the intermediate bend portion 654 may be simultaneous and to the same amount as the deflection of the proximal bend portion 658. The deflection mechanism may be configured to produce such simultaneous deflection of the intermediate and proximal bend portions 654, 658 and the same amount of the deflection of the intermediate and proximal bend portions 654, 658. The same amount of deflection of the intermediate and proximal bend portions 654, 658, although in opposite directions, may occur throughout a range of deflection of the intermediate and proximal bend portions 654, 658. An “S” shaped configuration of the elongate shaft 650 may result.

The intermediate and proximal bend portions 654, 658 may be deflected for a desired amount, and may be deflected such that the axis 670 of the intermediate extension portion 668 extends perpendicular with the axes 663, 666 of the respective distal and proximal extension portions 660, 664, and may continue to be deflected beyond such a position if desired, until the intermediate bend portion 654 contacts the proximal extension portion 664 if desired. FIG. 13D illustrates a deflection of the intermediate and proximal bend portions 654, 658 to an extent that the axis 670 extends perpendicular with the axes 663, 666. A lateral displacement of the end of the elongate shaft 650 and the capsule 106 has resulted, to a greater extent than shown in FIG. 13C. The end of the elongate shaft 650 and the capsule 106 has retracted to a greater extent than shown in FIG. 13C.

In examples, the bend portions 652, 654, 658 may be returned to their original undeflected positions for withdrawal of the elongate shaft 650 from a deployment site.

The deflection of the intermediate and proximal bend portions 654, 658 may provide a reduced complexity of the movement of the position of the distal end of the elongate shaft 650 and the capsule 106 than in an example shown in FIG. 12C. For example, a user may determine that an angular deflection of the capsule 106 relative to an axis of a heart valve may occur via the distal bend portion 652, and a lateral displacement of the capsule 106 may occur via the deflection of the intermediate and proximal bend portions 654, 658. Improved positioning of the distal end of the elongate shaft 650 and the capsule 106 may result during a deployment procedure. The direction that the distal bend portion 652 deflects in may also be the same upon the deflection of the intermediate and proximal bend portions 654, 656, whereas in an example shown in FIG. 12C the deflection of the bend portion 602 varies the direction that the bend portion 600 deflects in. The deflection may be utilized to centralize a position within a left or right atrium relative to an implantation site such as a tricuspid or mitral valve.

FIG. 13E illustrates a cross sectional view of the elongate shaft 650 showing components of a deflection mechanism that may be utilized to deflect the bend portions 652, 654, 658. The deflection mechanism, for example, may include a shaft 671 that may be positioned within an outer sheath 672 of the elongate shaft 650. The shaft 671 may include a distal portion 674, an intermediate portion 676, and a proximal portion 678. The position of the distal portion 674 may correspond to the distal bend portion 652, the position of the intermediate portion 676 may correspond to the intermediate bend portion 654, and the position of the proximal portion 678 may correspond to the proximal bend portion 658.

The deflection mechanism may include a first pull tether 680 or pull wire that may be configured to deflect the distal bend portion 652. The first pull tether 680, for example, may have a distal end 682 that may couple to a connection point on the shaft 671. The connection point may be positioned on a distal connector body 684 such as a distal connector ring or other structure of the shaft 671. The first pull tether 680 may extend proximally through an intermediate connector body 686 and a proximal connector body 688 of the shaft 671. For example, the first pull tether 680 may pass through a tether lumen 690 that may pass through the intermediate connector body 686 and the proximal connector body 688. A proximal end 692 of the first pull tether 680 may couple to a pull body 694 that may be configured to be moved to produce tension in the first pull tether 680 to retract the first pull tether 680, and release tension to allow the first pull tether 680 to advance distally.

The deflection mechanism may include a second pull tether 696 or pull wire that may be configured to deflect the intermediate bend portion 654. The second pull tether 696, for example, may have a distal end 698 that may couple to a connection point on the shaft 671. The connection point may be positioned on an intermediate connector body 686 such as an intermediate connector ring or other structure of the shaft 671. The second pull tether 696 may extend proximally through a proximal connector body 688 of the shaft 671. For example, the second pull tether 696 may pass through a tether lumen 700 that may pass through the proximal connector body 688. A proximal end 702 of the second pull tether 696 may couple to a pull body 704 that may be configured to be moved to produce tension in the second pull tether 696 to retract the second pull tether 696, and release tension to allow the second pull tether 696 to advance distally.

The position of the second pull tether 696 relative to the first pull tether 680 may be offset circumferentially to produce a desired direction of deflection of the distal bend portion 652 relative to the intermediate bend portion 654. The circumferential offset of the second pull tether 696 relative to the first pull tether 680 may be ninety degrees or may be another amount as desired. FIG. 13F, for example, illustrates a cross sectional view of the elongate shaft 650 transverse to the central axis of the elongate shaft 650 (along line 13F-13F in FIG. 13E). The circumferential position of the first pull tether 680 is shown. FIG. 13G illustrates a cross sectional view of the elongate shaft 650 along line 13G-13G in FIG. 13G. The circumferential position of the second pull tether 696 relative to the first pull tether 680 is shown.

The deflection mechanism may include a third pull tether 706 or pull wire that may be configured to deflect the proximal bend portion 658. The third pull tether 706, for example, may have a distal end 708 that may couple to a connection point on the shaft 671. The connection point may be positioned on a proximal connector body 688 such as a proximal connector ring or other structure of the shaft 671. The pull tether 706 may extend proximally to a proximal end 710 of the pull tether 706 that may couple to the pull body 704 that may be configured to be moved to produce tension in the pull tether 706 to retract the pull tether 706, and release tension to allow the pull tether 706 to advance distally. The proximal connector body 688 may correspond to the position of the intermediate extension portion 668 and the intermediate connector body 686 may correspond to the position of the distal extension portion 660.

The position of the third pull tether 706 relative to the first and second pull tethers 680, 696 may be offset circumferentially to produce a desired direction of deflection of the proximal bend portion 658 relative to the distal and intermediate bend portions 652, 654. The circumferential offset of the third pull tether 706 relative to the first pull tether 680 may be ninety degrees or may be another amount as desired. FIG. 13H, for example, illustrates a cross sectional view of the elongate shaft 650 transverse to the central axis of the elongate shaft 650 (along line 13H-13H in FIG. 13E). The circumferential position of the third pull tether 706 is shown relative to the first and second pull tethers 680, 696.

The third pull tether 706 may extend parallel with the second pull tether 696 at a position on the elongate shaft 671 that is opposed to the position of the second pull tether 696. The third pull tether 706 may couple to the proximal connector body 688 at a position, and the second pull tether 696 may couple to the intermediate connector body 686 at a position that is opposed to the position of connection of the third pull tether 706 with the proximal connector body 688. As such, the pull of the second pull tether 696 may be co planar with the pull of the third pull tether 706, yet may result in rotation of the respective intermediate and proximal bend portions 654, 658 in opposite directions. The rotation of the intermediate and proximal bend portions 654, 658 may be in a plane that is transverse to the plane of rotation of the distal bend portion 652.

The proximal ends 702, 710 of the respective second and third pull tethers 696, 706 may couple to the pull body 704 to allow the pull body 704 to retract both pull tethers 696, 706 simultaneously. The retraction of the pull body 704 may retract the second and third pull tethers 696, 706 by the same amount, causing the same amount of rotation of the bend portions 654, 658 as shown in FIGS. 13C and 13D for example. A distal advancement of the pull body 704 may reduce the deflection of the intermediate and proximal bend portions 654, 658 and may straighten the intermediate and proximal bend portions 654, 658 as desired. As such, deflection of both intermediate and proximal bend portions 654, 658 may be controlled simultaneously through control of the pull body 704. The distal bend portion 652 may be controlled independently via control of the pull body 694.

In examples, the shaft 671 may include one or more flex cuts to allow for deflection of the bend portions 652, 654, 658 in a desired manner. For example, the distal bend portion 652 may include one or more flex cuts 712 configured to allow the bend portion 652 to deflect. The flex cuts 712 may be positioned on the shaft 671 and may extend circumferentially. The flex cuts 712 may have an arcuate shape and may be spaced from each other axially. The flex cuts 712 may be positioned on a side of the shaft 671 that the pull tether 680 may extend along.

The intermediate bend portion 654 may include one or more flex cuts 714 configured to allow the intermediate bend portion 654 to deflect, and the proximal bend portion 658 may include one or more flex cuts 716 configured to allow the proximal bend portion 658 to deflect. The flex cuts 714 of the intermediate bend portion 654 may be positioned on a portion of the shaft 671 that is opposed to the position of the flex cuts 716 of the proximal bend portion 658. The second pull tether 696 may extend along the flex cuts 714 and the third pull tether 706 may extend along the flex cuts 716. The flex cuts 714, 716 may be configured to allow for a desired deflection of the bend portions 654, 658.

In examples, the tether lumens 690, 700 may comprise compression bodies or compression coils that may apply a distal compression, to reduce the possibility of undesired flexure of a bend portion 654, 658 while tension is produced in one of the pull tethers 680, 696, 706. For example, as tension is produced in the first pull tether 680, a compressive force applied to the tether lumen 690 may reduce the possibility of undesired deflection of the intermediate bend portion 654.

In examples, the outer sheath 672 may extend to the capsule 106, and may be utilized to retract the capsule 106 during deployment of the implant. In examples, the outer sheath 672 may comprise the outer sheath assembly 22 discussed herein. In examples, the shaft 671 may comprise the rail assembly 20 discussed herein and may be utilized in a similar manner as the rail assembly 20. For example, one or more other assemblies may be configured to be advanced or retracted relative to the shaft 671 to vary depth during a deployment operation of the implant. The shaft 671 may be utilized with one or more other assemblies disclosed herein, including an outer sheath assembly 22, an inner shaft assembly 18, a nose cone assembly 31, and/or a mid shaft assembly 21 as disclosed herein.

In examples, the pull bodies 694, 704 may be positioned within a handle of a delivery system, such as a handle 14 discussed in regard to FIGS. 10 and 11. The pull body 694, for example, may be configured to be operated by a distal knob 206, and the pull body 704 may be configured to be operated by a proximal knob 208, among other forms of operation. The pull body 704 may be configured to be retracted within a handle to retract the pull tethers 696, 706 simultaneously.

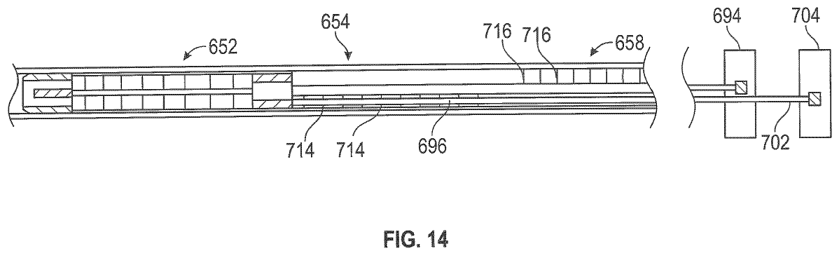

In examples, other forms of deflection mechanisms may be utilized as desired. FIG. 14 illustrates an example in which use of a third pull tether 706 may be excluded, and the second pull tether 696 may be utilized to deflect both the intermediate and proximal bend portions 654, 658. The second pull tether 696, for example, may extend along the intermediate and proximal bend portions 654, 658, with tension in the second pull tether 696 producing deflection of both of the intermediate and proximal bend portions 654, 658. The flex cuts 714, 716 may be configured to produce a desired opposite direction of rotation of the respective intermediate and proximal bend portions 654, 658. In examples, at least one pull tether may be configured to deflect the intermediate and proximal bend portions 654, 658. In examples, at least one pull tether may be configured to deflect the distal bend portion 652. In examples, the number of pull tethers utilized for each bend portion 652, 654, 658 may be varied as desired. For example, double pull tethers, or triple pull tethers for each bend portion 652, 654, 658 may be utilized as desired. Various other modifications of the delivery system may be utilized as desired.

The features of the examples of FIGS. 13A-14 may be utilized solely or in combination with any other example disclosed herein.

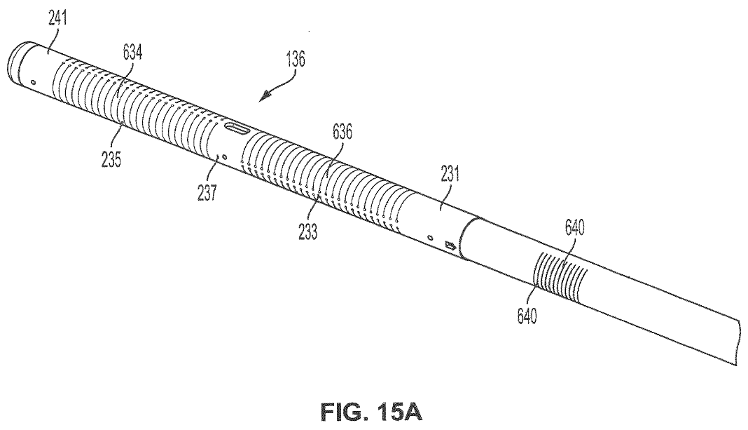

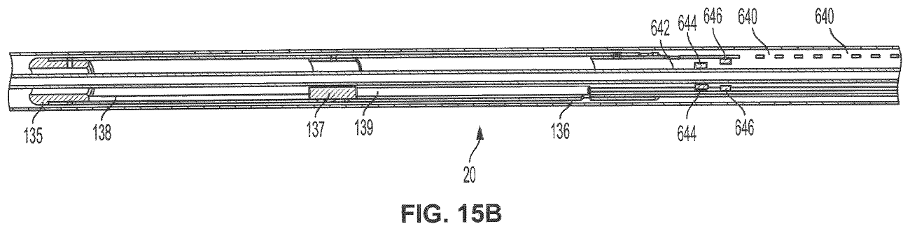

FIGS. 15A-B illustrate an example of a deflection mechanism including cuts 640 in a portion of the elongate shaft 12 and a pull shaft 642 that may be retracted to cause the elongate shaft 12 to deflect at the location of the cuts 640. Referring to FIG. 15A, the cuts 640 may be positioned on the rail shaft 132 at a desired location. Such a location may be proximal the rail hypotube 136 or bend portions 634, 636 of the rail shaft 132. For example, as shown in FIG. 15A, the cuts 640 may be proximal the uncut (or unslotted) hypotube section 231. The bend portion 634 may correspond to the bend portion 600 or 652, and the bend portion 636 may correspond to the bend portion 602 or 654.

The cuts 640 may have a configuration that biases the rail shaft 132 to deflect at the cuts 640 and in a direction that is away from the direction that the bend portion 634 has deflected the distal end of the elongate shaft 12.

Referring to FIG. 15B, a cross sectional view of the rail shaft 132 is shown. A deflection mechanism may include the inner shaft or pull shaft 642, which may be positioned within the rail shaft 132. The pull shaft 642 may be positioned between the rail shaft 132 and an inner shaft such as the inner shaft assembly 18 or the nose cone assembly 31. In other examples, the inner shaft or pull shaft 642 may be provided in other locations.

The inner shaft or pull shaft 642 may include a stopper 644 coupled thereto. The rail shaft 132, and particularly the portion of the rail shaft 132 distal the cuts 640 may include a stopper 646. The deflection mechanism may be configured that as the pull shaft 642 is drawn proximally, the stopper 644 contacts the stopper 646 and applies a proximal force to the rail shaft 132 and particularly the portion of the rail shaft 132 including the cuts 640. The cuts 640, providing a biased direction of deflection, may cause the rail shaft 132 and accordingly the elongate shaft 12 to deflect in this direction of deflection, which is in a direction that is opposed to the direction that the bend portion 634 has deflected the distal end of the elongate shaft 12. The pull shaft 642 may then be moved distally to reduce the force between the stoppers 644, 646 to cause the rail shaft 132 to straighten. FIG. 15B shows the stoppers 644, 646 separate from each other, however, the inner shaft or pull shaft 642 may be drawn proximally for the stoppers 644, 646 to contact each other.

A single pull shaft 642 is shown in FIG. 15B, however multiple pull shafts may be utilized in other examples as desired. For example, if four equally spaced pull shafts (spaced 90° from each other) with corresponding stoppers are utilized, then a combination of movement of the pull shafts may provide a variety of directions of deflection of the elongate shaft 12. The cut pattern may be provided such that a variety of directions of deflection are possible. Other configurations may be utilized to vary the direction of deflection of the elongate shaft 12. The one or more pull shafts accordingly may be configured to deflect the elongate shaft 12 to deflect the bend portions 634, 636 towards a direction that is opposed to the direction that the bend portion 634 has deflected the distal end of the elongate shaft 12, which may include a direction that is directly opposite the direction that the bend portion 634 has deflected the distal end of the elongate shaft 12 (at 180° degrees) and a variety of other directions that are in between and include direct opposition (at 180° degrees) and a perpendicular direction (at 90°) (e.g., 135°, among others).

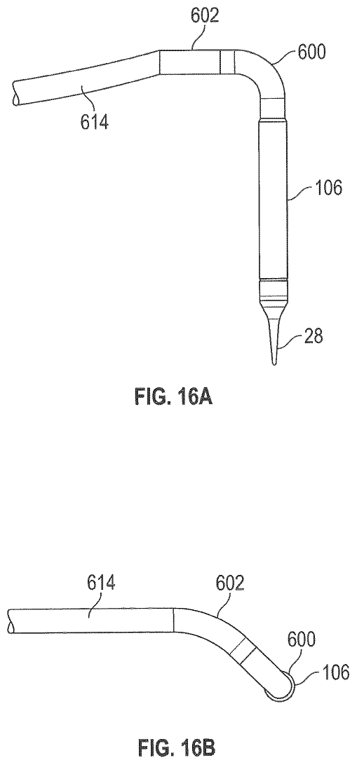

FIGS. 16A-B illustrate an external view of the examples of FIGS. 15A-15B. As shown in FIG. 16A, the bend portion 600 may deflect the distal end of the elongate shaft 12 to a direction. The deflection mechanism shown in FIGS. 15A-15B may deflect the proximal portion 614 of the elongate shaft 12 to deflect the bend portion 600 towards a direction that is opposed to the direction of the distal end of the elongate shaft 12. FIG. 16B illustrates that the bend portions 600, 602 may continue to operate to deflect the respective distal portions of the elongate shaft 12, with the distal bend portion 600 configured to deflect in a vertical plane, and the proximal bend portion 602 deflecting in a horizontal plane.

In examples, a benefit of a configuration of the pull shaft 642 and the stopper 646 on the rail shaft 132 is that as a depth knob 212 shown in FIGS. 10 and 11, for example, is rotated to reduce the depth of the pull shaft 642, the proximal portion 614 of the elongate shaft will deflect as shown in FIG. 16A. Thus, a user attempting to reduce the depth of the capsule 106 relative to an implantation site, for example, will be able to deflect the capsule 106 away from the implantation site upon an attempt to reduce the depth of the capsule 106. Such an operation may be beneficial during an implantation procedure, as a user may prefer that the capsule 106 continues to deflect away from an implantation site upon use of the depth knob 212, as the user was already attempting to retract the capsule 106 from the implantation site.

The deflection mechanisms may be utilized to provide for additional or varied movement of the elongate shaft 12. Such additional or varied movement may be desired for a variety of reasons, which may include a variety of patient anatomies to be navigated with the distal end of the elongate shaft 12 or varied uses of the elongate shaft 12.

The deflection mechanisms may be utilized to move the elongate shaft 12 for delivery of a replacement heart valve, which may include a replacement tricuspid valve. Although many of the examples herein are discussed in regard to a replacement tricuspid valve, the deflection mechanisms may be utilized for a variety of other implementations including delivery of mitral replacement valves, or aortic or pulmonary valves, or for valve repair procedures, including tricuspid or mitral valve repair or aortic or pulmonary valve repair.

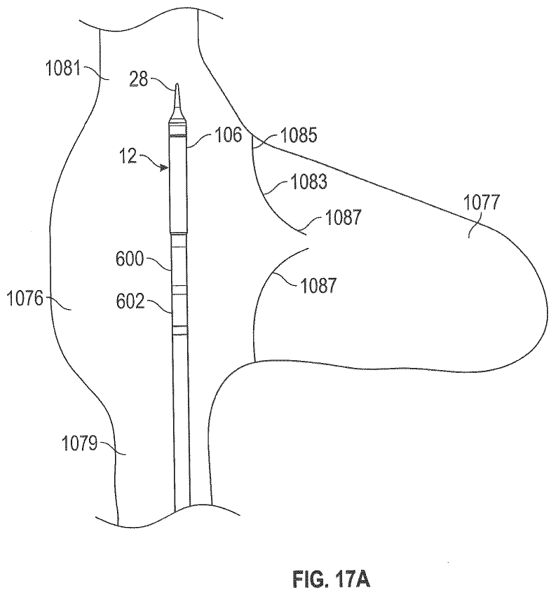

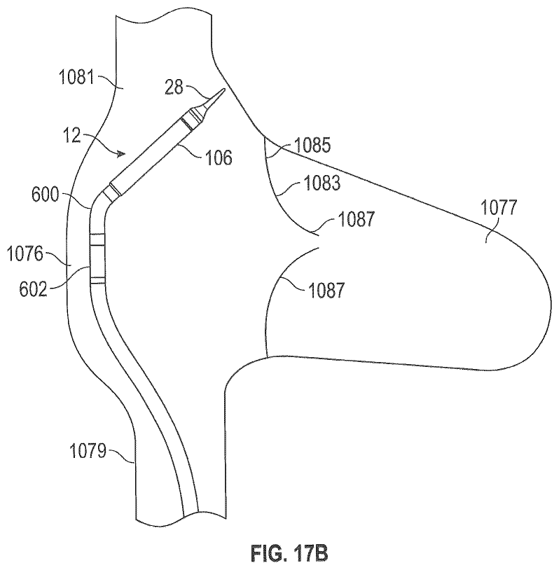

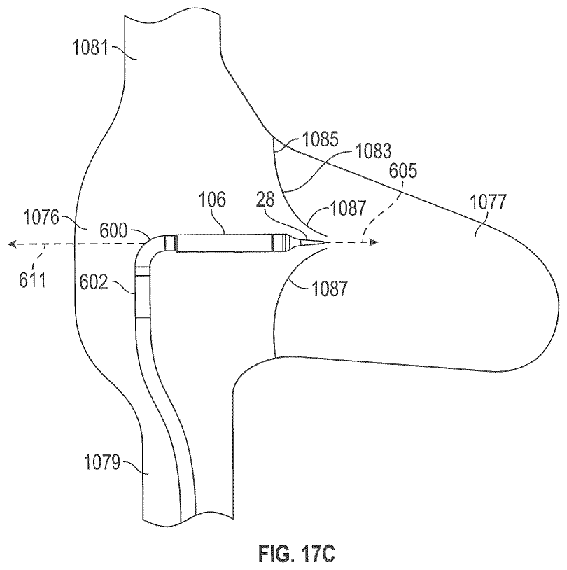

FIGS. 17A-19 illustrate a use of the elongate shaft 12 to treat a patient's tricuspid valve. The elongate shaft 12 may be passed into the patient's body in an endovascular manner, which may include percutaneous entry of the patient's vasculature. For example, the elongate shaft 12 may be entered into the ipsilateral femoral vein and advanced toward the right atrium 1076. Other entry methods may be utilized in other examples, including a transjugular approach, or other approaches including transapical approaches.

As shown in FIG. 17A, the elongate shaft 12 may be advanced through the inferior vena cava 1079 to approach or reach the right atrium 1076 of the patient's heart. The right ventricle 1077, the tricuspid valve 1083 including tricuspid valve leaflets 1087, the tricuspid valve annulus 1085, and the superior vena cava 1081 are also shown.

The delivery system may include use of the deflection mechanisms discussed herein. The elongate shaft 12 may be advanced towards the right atrium 1076, with the distal end of the elongate shaft 12 to be deflected such that the capsule 106 and thus the implant retention area 16 are oriented to deploy the implant contained therein to the tricuspid valve 1083 in the desired manner. As represented in FIG. 17A, the distal end of the elongate shaft 12 may require deflection to a direction towards the tricuspid valve 1083, to align the distal end of the elongate shaft 12 and the capsule 106 (and the deployment port at the distal end of the capsule for the implant to be deployed from) with the central axis of the tricuspid valve 1083. For other methods of deployment, other directions of deflection may be desired.

The bend portions 600, 602 may be utilized to deflect the distal end of the elongate shaft 12 to the desired direction. The bend portions 600, 602 may be configured to deflect the distal end of the elongate shaft in perpendicular planes, to provide two planes of deflection. The bend portions 600, 602 may be configured similarly as shown in FIG. 6C, with the proximal bend portion 602 configured to deflect the distal portions of the elongate shaft 12 in a rightward (or anterior) direction relative to a downward (or ventricular) direction of deflection of the distal bend portion 600. Such a configuration may account for the position of the tricuspid valve 1083 relative to the inferior vena cava 1079 within a human heart.

Additional movement, however, may be provided by the deflection mechanisms disclosed herein. Such a deflection may include deflecting the proximal portion of the elongate shaft 12 and the bend portions 600, 602 in an atrial direction (or providing a height from the tricuspid valve 1083). The capsule 106 and distal end of the elongate shaft 12 may also be deflected in an atrial direction (or providing a height from the tricuspid valve 1083).

The deflection mechanism may be utilized to account for a geometry of the patient's anatomy, which may include the geometry of the right atrium 1076, the size and relative position of the tricuspid valve 1083, and the geometry of the inferior vena cava 1079. For example, as shown in FIG. 17A, the distance of the bend portion 600 to the distal end of the elongate shaft 12 may be such that the bending radius of the elongate shaft 12 distal the bend portion 600 is too large to properly direct the distal end of the elongate shaft 12 to the tricuspid valve 1083, depending on the geometry of the patient's right atrium 1076. The deflection mechanism accordingly may be utilized to deflect the bend portion 600 towards a direction opposed to the direction that the bend portion 600 deflects the distal end of the elongate shaft 12.

Referring to FIG. 17B, a deflection mechanism as discussed in regard to FIGS. 15A and 15B may be utilized to increase the height of the capsule 106 by deflecting a proximal portion of an elongate shaft 12. The deflection of the proximal portion of the elongate shaft 12 may occur wholly or partially (at least partially) within the patient's inferior vena cava 1079. The deflection may move the bend portions 600, 602 to create height from the tricuspid valve 1083 in a direction away from the tricuspid valve. As such, the distal end of the elongate shaft 12 may have greater clearance space for the bend portion 600 to deflect the distal end of the elongate shaft 12 towards the tricuspid valve 1083. As shown in FIG. 17B, the deflection mechanism may form a curve of the proximal portion of the sheath, although other forms of deflection may result. The bend portion 600 has begun deflection of the distal end of the elongate shaft 12 in FIG. 17B.

Referring to FIG. 17C, the bend portion 600 has deflected the distal end of the elongate shaft 12 to a direction 605. The direction may be aligned with the axis of the tricuspid valve 1083 or otherwise may be directed in a desired orientation. The deflection mechanism has deflected the proximal portion of the elongate shaft 12 to deflect the bend portion 600 in a direction 611 that is opposed to the direction 605. As such, the capsule 106 has increased height from the tricuspid valve 1083, to allow for deployment of the implant contained therein.

The deflection mechanisms may be utilized to deflect the proximal portion of the elongate shaft 12 in one or more planes that are not perpendicular to the plane that the bend portion 600 deflects the distal end of the elongate shaft 12.

In examples, a configuration of a delivery system as shown in FIGS. 13A-14 may be utilized to direct the elongate shaft in a desired orientation relative to an implantation site. A deflection mechanism as discussed in regard to FIGS. 13A-14, for example, may be utilized to centralize or align the capsule 106 and distal end of the elongate shaft 12 with an implantation site, such as an annulus of a heart valve (e.g., a tricuspid or mitral annulus). The features of the delivery system as shown in FIGS. 13A-14 may be combined with features as shown in FIGS. 15A-16B if desired.

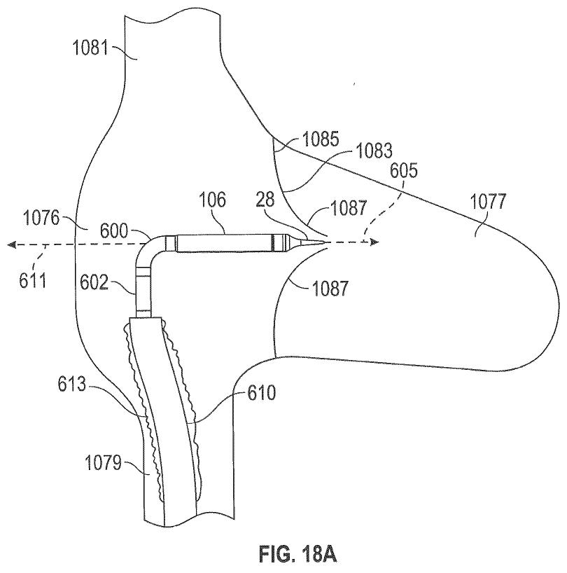

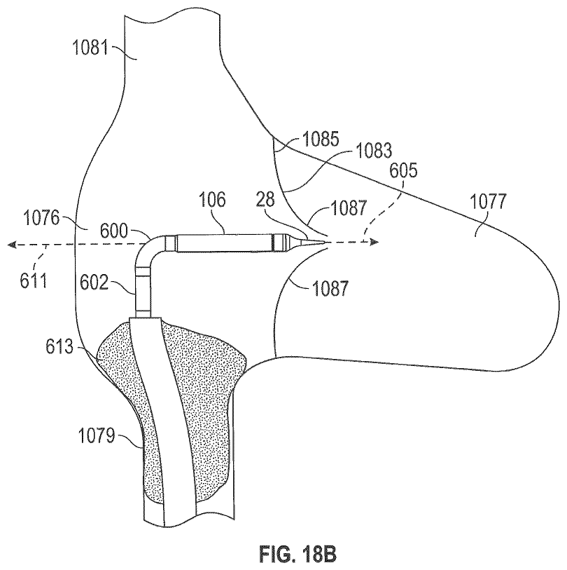

In examples, an inflatable body 613 may be provided that may extend radially outward from the elongate shaft. Referring to FIG. 18A, the inflatable body 613 may be positioned upon a sheath 610 that may extend over the elongate shaft, although in examples the inflatable body 613 may be integral with the elongate shaft. The sheath 610 may be slidable with respect to the elongate shaft 12 in examples.

The inflatable body 613 may be configured to inflate to expand radially outward from the elongate shaft 12. The inflatable body 613, upon expansion, may press against an inner surface of the patient's vasculature to aid in securing the elongate shaft 12 in a desired position during a deployment procedure. FIG. 18A, for example, illustrates the inflatable body 613 in a deflated state within the patient's vasculature, for example, the inferior vena cava 1079 and the right atrium 1076. The elongate shaft 12 is shown in position for deployment of an implant relative to a tricuspid valve 1083.

The inflatable body 613 may be inflated to have an increased diameter as shown in FIG. 18B for example. The inflatable body 613 may press against the inner surface of the vasculature, such as the inner surface of the interior vena cava 1079 and the right atrium 1076. The pressure of the inflatable body 613 against such surfaces may secure the elongate shaft 12 from undesired deflection upon deployment of the implant.

At a desired point, the inflatable body 613 may be deflated and withdrawn from the patient's vasculature. For example, following a deployment of the implant, the inflatable body 613 may be deflated and withdrawn. The features of FIGS. 18A-18B may be utilized with a delivery system as shown in FIGS. 13A-14.

In examples, the inflatable body 613 may include a channel to allow for local fluid flow to pass therethrough. For example, the channel may allow a patient's blood to flow through the inflatable body 613 and through the patient's vasculature during an implantation procedure.

The features of the examples of FIGS. 18A-18B may be utilized solely or in combination with any other example disclosed herein.

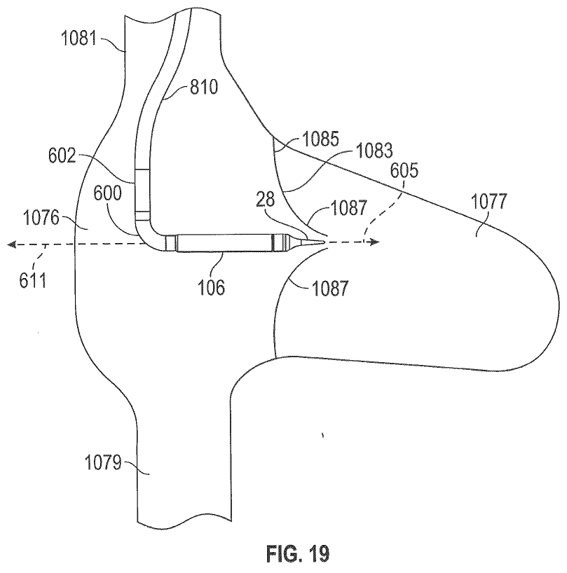

In examples, other approaches to a deployment site may be utilized. FIG. 19, for example, illustrates use of the deflection mechanism in an approach from the superior vena cava 1081. The approach may be a transjugular approach, or via another entry point into the patient's body. The bend portions 600, 602 may be configured similarly as shown in FIG. 6B, with the proximal bend portion 602 configured to deflect the distal portions of the elongate shaft 12 in a leftward (or posterior) direction relative to a downward (or ventricular) direction of deflection of the distal bend portion 600. Such a configuration may account for the position of the tricuspid valve 1083 relative to the superior vena cava 1081 within a human heart. A method may include passing a delivery apparatus for an implant into a patient's right atrium.

The deflection mechanism, similarly as shown in FIGS. 17A-C, may deflect the proximal portion of the elongate shaft 12 to deflect the bend portion 600 in a direction 611 that is opposed to the direction 605 that the bend portion 600 has deflected the distal end of the elongate shaft 12.

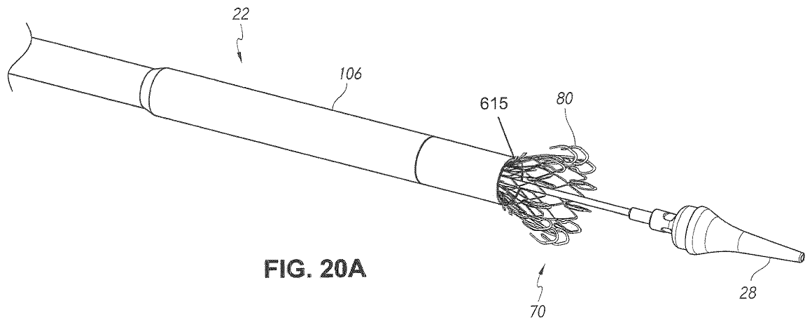

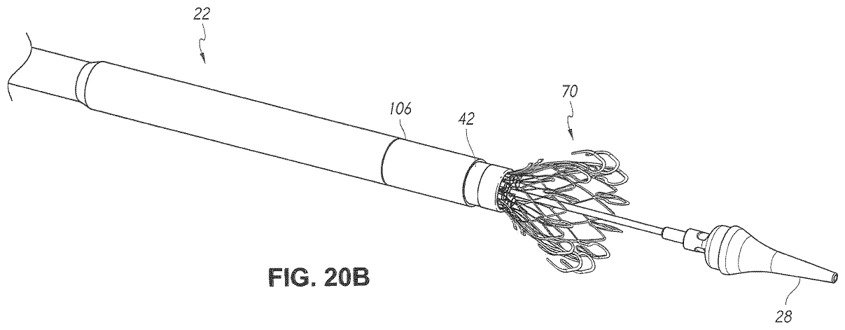

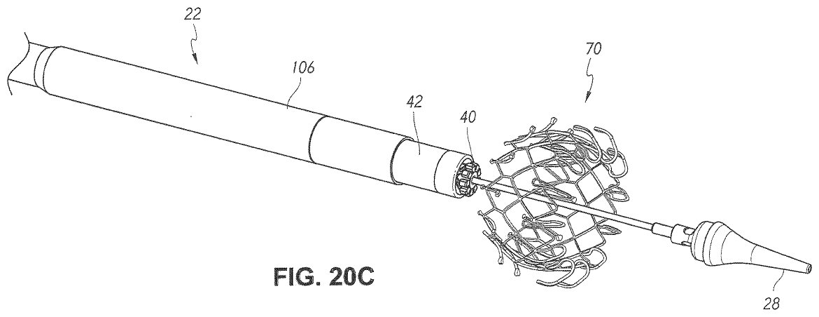

An implant contained within the capsule 106 may be deployed to be positioned within the tricuspid valve annulus 1085, to replace the native tricuspid valve 1083. Referring to FIGS. 20A-C, upon the distal end of the elongate shaft 12 being oriented as desired relative to the native tricuspid valve 1083, a release mechanism may be utilized to deploy an implant such as the implant 70 shown in FIG. 3E, among other forms of implants disclosed herein, from a deployment port 615 at the distal end of the capsule 106. A height of the deployment port 615 relative to the valve may be varied by deflecting the delivery apparatus within an inferior vena cava or a superior vena cava. FIGS. 20A-C illustrate the release mechanism of the delivery system 10. During the initial insertion of the implant 70 and the delivery system 10 into the body, the implant 70 can be located within the system 10, similar to as shown in FIG. 2A. The distal end 303 of the implant 70, and specifically the distal anchors 80, are restrained within the capsule 106 of the outer sheath assembly 22, thus preventing expansion of the implant 70. Similar to what is shown in FIG. 2A, the distal anchors 80 can extend distally when positioned in the capsule. The proximal end 301 of the implant 70 is restrained within the capsule 106 and within a portion of the inner retention member 40 and thus is generally constrained between the capsule 106 and the inner retention member 40.

Once the implant 70 is loaded into the delivery system 10, a user can thread a guide wire into a patient to the desired location. The guide wire passes through the lumen of the nose cone assembly 31, and thus the delivery system 10 can be generally advanced through the patient's body following the guide wire. The delivery system 10 can be advanced by the user manually moving the handle 14 in an axial direction. In some examples, the delivery system 10 can be placed into a stand while operating the handle 14 controls.

Once generally in heart, the user can begin the steering operation of the rail assembly 20, and particularly the bend portions 600, 602 using the distal pull wire knob 206 and/or the proximal pull wire knob 208. By turning either of the knobs, the user can provide flexing/bending of the rail assembly 20 (either on the distal end or the proximal end), thus bending the distal end of the delivery system 10 in one, two, or more locations into the desired configuration. As discussed above, the user can provide multiple bends in the rail assembly 20 to direct the delivery system 10 towards the tricuspid valve. In particular, the bends of the rail assembly 20 can direct a distal end of the delivery system 10, and thus the capsule 106, along the center axis passing through the native tricuspid valve and towards the tricuspid valve. Thus, when the outer sheath assembly 22, mid shaft assembly 21, inner shaft assembly 18, and nose cone assembly 31 are together advanced over the rail assembly 20 with the compressed implant, the capsule 106 proceeds directly in line with the axis for proper release of the implant 70. A configuration of a delivery system as shown in FIGS. 13A-14 may be utilized to direct the elongate shaft in a desired orientation relative to an implantation site.

The user may utilize the deflection mechanism as shown in FIGS. 15A-15B, which may create height from the native tricuspid valve or may otherwise orient the distal end of the elongate shaft 12 as desired. The height of a bend portion of the elongate shaft 12 may be varied from the tricuspid valve.

The system 10 can be positioned to a particular location in a patient's body, such as at the native tricuspid valve, through the use of the bend portions and deflection mechanisms discussed herein or other techniques.

The user can also rotate and/or move the handle 14 itself in a stand for further fine tuning of the distal end of the delivery system 10. The user can continually turn the proximal and/or distal pull wire knobs 208/206, as well as moving the handle 14 itself, to orient the delivery system 10 for release of the implant 70 in the body. The user can also further move the other assemblies relative to the rail assembly 20, such as proximally or distally. Upon the distal end of the elongate shaft 12 being oriented as desired, the user may rotate the depth knob 212. As discussed, rotation of this knob 212 together advances the inner shaft assembly 18, mid shaft assembly 21, outer sheath assembly 22, and nose cone assembly 31 over/through the rail assembly 20 while the implant remains in the crimped configuration within the implant retention area 16. Due to the rigidity of, for example, either the inner shaft assembly 18, the mid shaft assembly 21, and/or the outer sheath assembly 22, these assemblies proceed straight forward in the direction aligned by the rail assembly 20.

Once in the release position, the user can rotate the outer sheath knob 210, which individually translates the outer sheath assembly 22 (and thus the capsule 106) with respect to the other assemblies, in particular the inner assembly 18, in a proximal direction towards the handle 14 as shown in FIG. 20A. By doing so, a distal end 303 of an implant 70 is uncovered in the body, allowing for the beginning of expansion. At this point, the distal anchors 80 can flip proximally and the distal end 303 begins to expand radially outwardly. For example, if the system 10 has been delivered to a native tricuspid valve location, the distal anchors 80 expand radially outwardly within the right ventricle. The distal anchors 80 can be located above the papillary heads, but below the tricuspid valve annulus and tricuspid valve leaflets.

In some examples, the distal anchors 80 may contact and/or extend between the chordae in the right ventricle, as well as contact the leaflets, as they expand radially. In some examples, the distal anchors 80 may not contact and/or extend between the chordae or contact the leaflets. Depending on the position of the implant 70, the distal ends of the distal anchors 80 may be at or below where the chordae connect to the free edge of the native leaflets.

As shown in the illustrated example, the distal end 303 of the implant 70 is expanded outwardly. It should be noted that the proximal end 301 of the implant 70 can remain covered by the outer retention ring during this step such that the proximal end 301 remains in a radially compacted state. At this time, the system 10 may be withdrawn proximally so that the distal anchors 80 capture and engage the leaflets of the tricuspid valve, or may be moved proximally to reposition the implant 70. For example, the assemblies may be proximally moved relative to the rail assembly 20. Further, the deflection mechanisms may be utilized to draw the elongate shaft 12 proximally relative to the tricuspid valve. Further, the system 10 may be torqued, which may cause the distal anchors 80 to put tension on the chordae through which at least some of the distal anchors may extend between. However, in some examples the distal anchors 80 may not put tension on the chordae. In some examples, the distal anchors 80 may capture the native leaflet and be between the chordae without any further movement of the system 10 after withdrawing the outer sheath assembly 22.

During this step, the system 10 may be moved proximally or distally to cause the distal or ventricular anchors 80 to properly capture the native tricuspid valve leaflets. This can be done by moving the outer sheath assembly 22, mid shaft assembly 21, inner shaft assembly 18, and nose cone assembly 31 with respect to the rail assembly 20. In particular, the tips of the ventricular anchors 80 may be moved proximally to engage a ventricular side of the native annulus, so that the native leaflets are positioned between the anchors 80 and the body of the implant 70. When the implant 70 is in its final position, there may or may not be tension on the chordae, though the distal anchors 80 can be located between at least some of the chordae.

The proximal end 301 of the implant 70 will remain in the outer retention ring 42 after retraction of the capsule 106. The capsule 106 may surround the implant retention area and be retracted proximally to deploy the implant. As shown in FIG. 20B, once the distal end 303 of the implant 70 is fully expanded (or as fully expanded as possible at this point), the outer retention ring 42 can be individually withdrawn proximally with respect to the other assemblies, in particular relative to the inner assembly 18, to expose the inner retention member 40, thus beginning the expansion of the proximal end 301 of the implant 70. For example, in a tricuspid valve replacement procedure, after the distal or ventricular anchors 80 are positioned between at least some of the chordae tendineae and/or engage the native tricuspid valve annulus, the proximal end 301 of the implant 70 may be expanded within the right atrium.

The outer retention ring 42 can be moved proximally such that the proximal end 310 of the implant 70 can radially expand to its fully expanded configuration as shown in FIG. 20C. The implant 70 may be deployed to the valve. After expansion and release of the implant 70, the inner shaft assembly 18, nose cone assembly 31, mid shaft assembly 21, and outer sheath assembly 22 can be simultaneously withdrawn proximally along or relative to the rail assembly 20 back to their original position. In some examples, they are not withdrawn relative to the rail assembly 20 and remain in the extended position. Further, the nose cone 28 can be withdrawn through the center of the expanded implant 70 and into the outer sheath assembly 22, such as by proximally translating the knob 216. The system 10 can then be removed from the patient.

In some examples, the implant 70 can be delivered under fluoroscopy so that a user can view certain reference points for proper positioning of the implant 70. Further, echocardiography can be used for proper positioning of the implant 70.

Reference is now made to FIG. 21 which illustrates a schematic representation of a portion of an example of a replacement heart valve (such as implant 70 shown in FIG. 3E) positioned within a native tricuspid valve of a heart 83. A portion of the native tricuspid valve is shown schematically and represents typical anatomy, including a right atrium 1076 positioned above an annulus 1085 and a right ventricle 1077 positioned below the annulus 1085. The right atrium 1076 and right ventricle 1077 communicate with one another through a tricuspid annulus 1085. Also shown schematically in FIG. 21 is a native tricuspid leaflet 1087 having chordae tendineae 1089 that connect a downstream end of the tricuspid leaflet 1087 to the papillary muscle of the right ventricle 1077. The portion of the implant 70 disposed upstream of the annulus 1085 (toward the right atrium 1076) can be referred to as being positioned supra-annularly. The portion generally within the annulus 1085 is referred to as positioned intra-annularly. The portion downstream of the annulus 1085 is referred to as being positioned sub-annularly (toward the right ventricle 1077).

As shown in FIG. 21, the replacement heart valve (e.g., implant 70) can be positioned so that the tricuspid annulus 1085 is located between the distal anchors 80 and the proximal anchors 82. In some situations, the implant 70 can be positioned such that ends or tips of the distal anchors 80 contact the annulus 1085 as shown, for example, in FIG. 21. In some situations, the implant 70 can be positioned such that ends or tips of the distal anchors 80 do not contact the annulus 1085. In some situations, the implant 70 can be positioned such that the distal anchors 80 do not extend around the leaflet 1087.

As illustrated in FIG. 21, the replacement heart valve or implant 70 can be positioned so that the ends or tips of the distal anchors 80 are on a ventricular side of the tricuspid annulus 1085 and the ends or tips of the proximal anchors 82 are on an atrial side of the tricuspid annulus 1085. The distal anchors 80 can be positioned such that the ends or tips of the distal anchors 80 are on a ventricular side of the native leaflets beyond a location where chordae tendineae 1089 connect to free ends of the native leaflets. The distal anchors 80 may extend between at least some of the chordae tendineae 1089 and, in some situations such as those shown in FIG. 21, can contact or engage a ventricular side of the annulus 1085. It is also contemplated that in some situations, the distal anchors 80 may not contact the annulus 1085, though the distal anchors 80 may still contact the native leaflet 1087. In some situations, the distal anchors 80 can contact tissue of the right ventricle 1077 beyond the annulus 1085 and/or a ventricular side of the leaflets.

Although FIG. 21 shows deployment to a tricuspid annulus, other implantation sites may be utilized in examples including a mitral annulus, or an aortic or pulmonary annulus. Other implantation sites may be utilized in examples.

Upon deployment of the implant 70 as desired, the deflection mechanisms disclosed herein may be utilized to deflect the elongate shaft 12 to allow for removal of the elongate shaft 12 from the patient's heart.

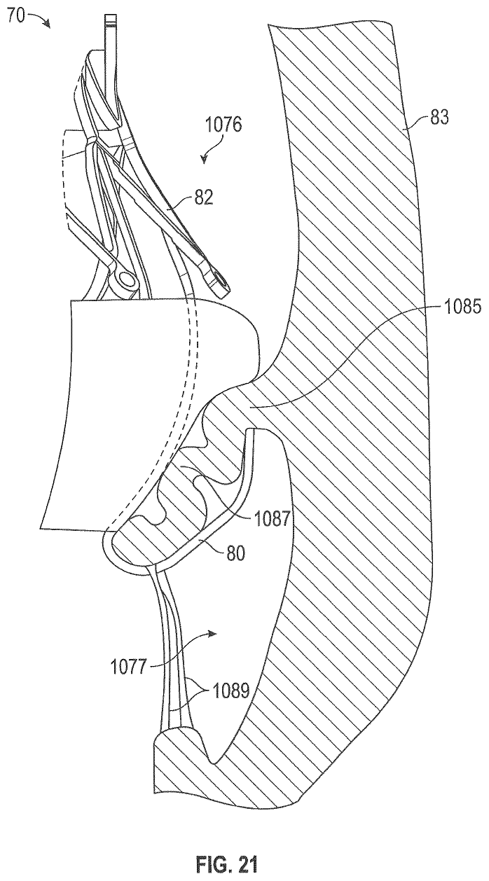

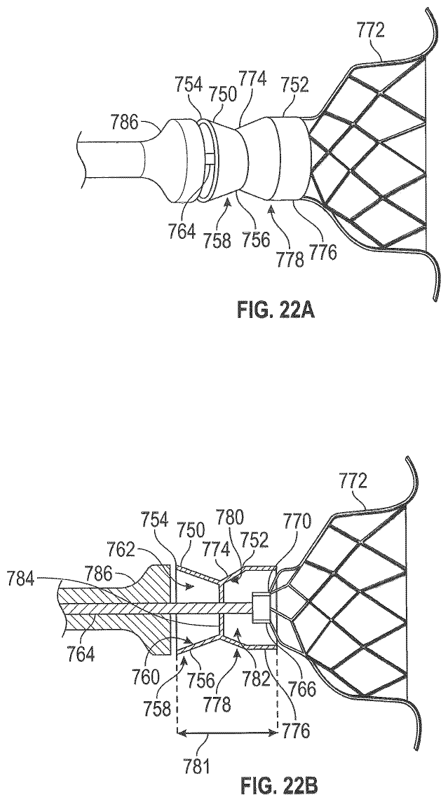

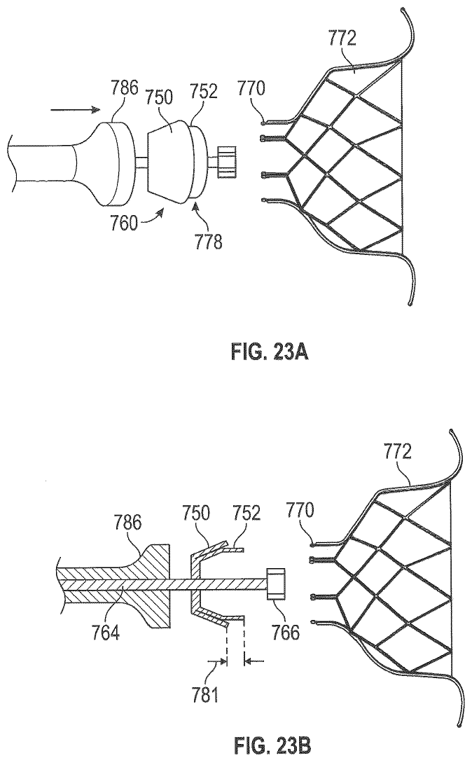

In examples, other forms of deployment mechanisms may be utilized as desired. FIGS. 22A-23B, for example, illustrate an example of a deployment mechanism including a diaphragm 750 that may be utilized for deployment of an implant from an elongate shaft of a delivery system. A retention body 752 may be provided that may be configured to retain an implant. The diaphragm 750 may extend proximally and may be configured to be moved distally to allow the retention body 752 to release from the implant.

Referring to FIGS. 22A and 22B, the diaphragm 750 may include a proximal end portion 754 and a distal end portion 756 and a length between the proximal end portion 754 and the distal end portion 756 of the diaphragm 750. The diaphragm 750 may include an outer surface 758 that may face radially outward and may include an inner surface 760 that may face radially inward and towards an interior cavity 762 of the diaphragm 750. The proximal end portion 754 may comprise an opening for the interior cavity 762.

The proximal end portion 754 of the diaphragm 750 may comprise a wide portion of the diaphragm 750 and the distal end portion 756 of the diaphragm 750 may comprise a narrow portion of the diaphragm 750. The wide portion of the diaphragm 750 may extend proximally from the narrow portion of the diaphragm 750. The diaphragm 750 may have a conical shape such as a conical frustum shape.

The diaphragm 750 may surround an inner shaft 764 that may extend distally to a coupling body 766 or member that may be configured to couple to the implant. The inner shaft 764 may pass through the diaphragm 750 and the retention body 752. The coupling body 766 may be configured to couple to end couplers 770 of the implant 772, similar to the operation of the inner retention member or inner retention ring 40 shown in FIGS. 2A and 2B for example.

The implant 772 may be configured similarly as examples of implants disclosed herein. For example, the end couplers 770 of the implant 772 may be configured similarly as the mushroom-shaped tabs 74 shown in FIG. 3E, or may have another configuration. The implant may comprise a self-expanding implant in examples. The frame of the implant 772 is shown in FIGS. 22A-23B, yet the implant may comprise an implant having features for full deployment of the implant, including prosthetic valve leaflets and any other feature of the implants shown in FIGS. 3A-3E for example.

The retention body 752 may include a proximal end portion 774 and a distal end portion 776 and a length therebetween. The retention body 752 may include an outer surface 778 facing opposite an inner surface 780. The outer surface 778 may face radially outward. The inner surface 780 may face an interior cavity 782 that the coupling body 766 may be positioned within. The retention body 752 may cover the coupling body 766. The retention body 752 may have a conical shape, such as a conical frustum shape, with the wide portion extending distally and the narrow portion extending proximally. Other configurations of retention bodies 752 may be utilized as desired.

The retention body 752 may be configured to apply a sufficient radially compressive force to the implant 772 to prevent the implant 772 from releasing from the coupling body 766 while the retention body 752 extends over the implant 772.

The proximal end portion 774 of the retention body 752 may be coupled to the distal end portion 756 of the diaphragm 750. The retention body 752 and the diaphragm 750 may include a coupling portion 784 that may couple the retention body 752 and the diaphragm 750 to the inner shaft 764 and may allow the retention body 752 and the diaphragm 750 to slide together along the inner shaft 764. The coupling portion 784, for example, may include an aperture between the retention body 752 and the diaphragm 750 that the inner shaft 764 may slide together along.

The retention body 752 and the diaphragm 750 may have a length 781 from the distal end of the retention body 752 to the proximal end of the diaphragm 750 in a configuration as shown in FIGS. 22A and 22B.

In examples, the diaphragm 750 may be configured to be flexible to allow the diaphragm 750 to move upon a compressive force being applied to the diaphragm 750. For example, upon a distal compressive force being applied to the diaphragm 750, the diaphragm 750 may invert and may overlay over the outer surface 778 of the retention body 752. The diaphragm 750 may invert upon being moved distally. In such a configuration, the inner surface 760 of the diaphragm 750 may face radially outward and the outer surface 758 may face radially inward. The wide portion of the diaphragm 750 may extend distally from the narrow portion of the diaphragm 750. FIGS. 23A and 23B, for example, illustrate such a configuration.

In examples, a pusher shaft 786 may be provided that may be configured to be advanced distally to move the diaphragm 750 distally. The pusher shaft 786 may be configured to apply a distal compressive force to the proximal end portion 754 of the diaphragm 750 to move the diaphragm 750. The pusher shaft 786, for example, may be configured to resist compression to apply the compressive force to the proximal end portion 754 of the diaphragm 750.

In operation, and in a configuration as shown in FIGS. 22A and B, the pusher shaft 786 may apply a force distally to the proximal end portion 754 of the diaphragm 750 to cause the diaphragm 750 to convey that force to the retention body 752. As such, the pusher shaft 786 may resist the retention body 752 from moving proximally and thus allowing the retention body 752 to remain in position over the coupling body 766, thus maintaining the coupling of the implant 772 to the coupling body 766.

At a desired point, the pusher shaft 786 may be advanced distally, applying the compressive force to the diaphragm 750 that causes the diaphragm 750 to compress and move distally. The diaphragm 750 may invert as shown in FIGS. 23A and 23B. The force to invert the diaphragm 750 may be greater than the force exerted on the diaphragm 750 by the implant as the implant attempts to self-expand. The movement of the diaphragm 750 may reduce the overall length of the diaphragm 750 and the retention body 752, thus producing space for the diaphragm 750 and the retention body 752 to slide proximally along the inner shaft 764. As such a length 781 from the proximal end of the diaphragm 750 to the distal end of the retention body 752 may be configured to reduce upon the diaphragm 750 being moved distally.

With the space for the diaphragm 750 and the retention body 752 to slide, the retention body 752 may retract proximally to release from the implant, as shown in FIGS. 23A and 23B for example. The retention body 752 may retract from the coupling body 766 upon the diaphragm 750 being moved distally. The implant 772, for example, may comprise a self-expanding implant such as a self-expanding prosthetic valve, which may apply a radially outward force that may push the retention body 752 proximally. Upon release of the implant 772, the components of the delivery system may be retracted from the implantation site.

A delivery system as shown in FIGS. 22A-23B may allow for release of an implant upon a distal compressive force being produced. Such a distal compressive force may reduce the use of tensile bodies in the delivery system that provide a retraction force to release an implant (such as a retraction force within a capsule surrounding an implant retention area). Various modifications of the delivery system may be provided as desired.

Components of the delivery system as shown in FIGS. 22A-23B may be utilized solely or in combination with features of other delivery systems disclosed herein. For example, the components of the delivery system shown in FIGS. 22A-23B may be utilized with other examples having a handle for operation of the delivery system. Proximal portions of the pusher shaft 786 and the inner shaft 764 for example, may be controlled at the handle to allow for release and operation of the delivery system. In examples, a capsule may surround the implant 772 to reduce the diameter of the implant 772 prior to deployment.

The features of the examples of FIGS. 22A-23B may be utilized solely or in combination with any other example disclosed herein.

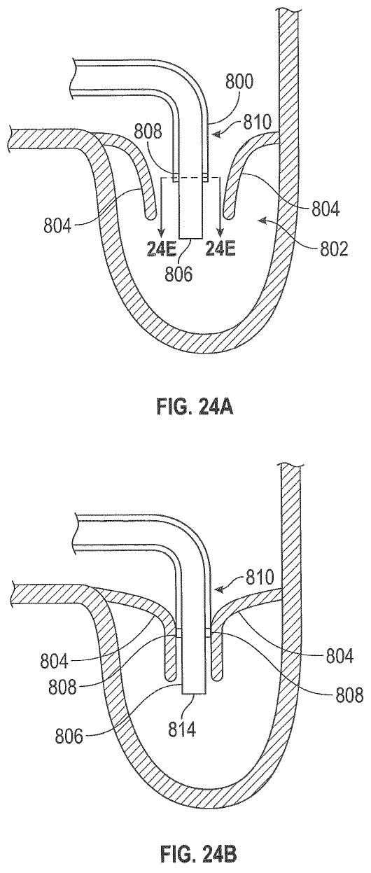

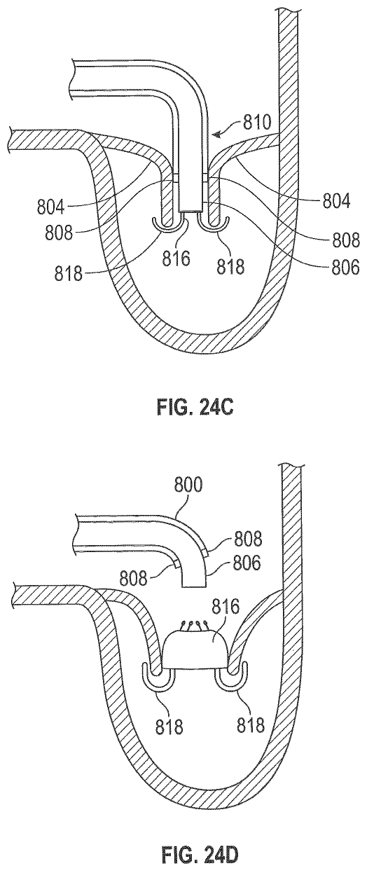

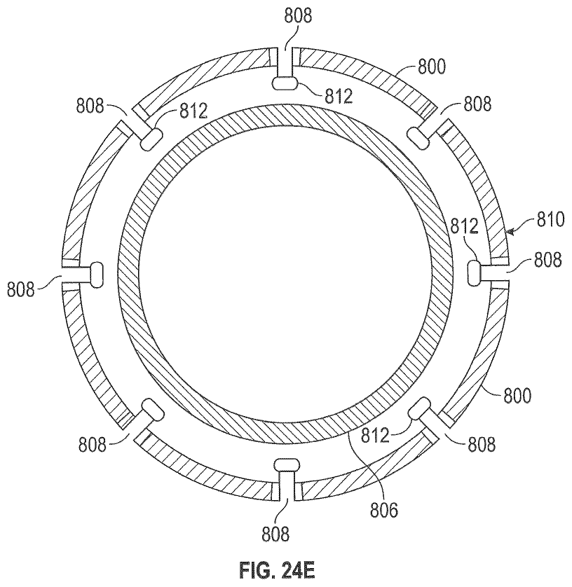

FIGS. 24A-24E illustrate an example of a delivery system including one or more suction ports. The delivery system may include an elongate shaft 800 that may be configured to be advanced to an implantation site, such as a native heart valve 802. The native heart valve 802 may include portions such as native heart valve leaflets 804 that may control opening and closing of the native heart valve 802.

The elongate shaft 800 may be flexible and configured to advance to a desired position relative to the implantation site. The elongate shaft 800 may include an implant retention area surrounded by a capsule 806 that may be configured to be retracted to at least partially deploy an implant from the implant retention area. In examples, the elongate shaft 800 may be configured similarly as other examples of elongate shafts disclosed herein. The elongate shaft 800, for example, may be deflectable to approach the implantation site in a desired manner.

The one or more suction ports 808 may be configured to extend along the elongate shaft 800 and in examples may be coupled to the elongate shaft 800. For example, the elongate shaft 800 in examples may include an outer surface 810 and the one or more suction ports 808 may be positioned on the outer surface 810.

FIG. 24E, for example, illustrates a cross sectional view of the elongate shaft 800 along line 24E-24E in FIG. 24A. A plurality of suction ports 808 may be provided that may be circumferentially spaced from each other. The plurality of suction ports 808 may be circumferentially spaced from each other on the outer surface 810 of the elongate shaft 800. The circumferential spacing of the suction ports 808 may allow the suction ports 808 to apply suction to a portion of a heart valve to draw the portion towards the elongate shaft 800. The suction may be applied about the outer surface 810 of the elongate shaft 800.

In examples, one or more suction conduits 812 may extend along the elongate shaft 800 and may be configured to provide suction for the one or more suction ports 808. The suction conduits 812, for example, may extend interior of the outer surface 810 of the elongate shaft 800 and a distal end of each may be coupled to a respective suction port 808. A proximal end of each of the suction conduits 812 may be coupled to a device, such as a vacuum device (e.g., a syringe or other form of vacuum device) for providing suction. The device may be positioned exterior of the patient's body and may be connected to the suction conduits 812 at a handle or other location as desired.

In examples, the suction ports 808 and the suction conduits 812 may not be coupled to an elongate shaft 800 and may comprise components separate from the elongate shaft 800 that may apply suction in a similar manner as the suction ports 808 coupled to the elongate shaft 800. For example, a separate suction port assembly may be advanced to a location within a patient's body, with the suction port assembly including the suction ports 808.

The one or more suction ports 808 may be configured to apply suction to native heart valve leaflets 804 to draw the native heart valve leaflets 804 radially inward. For example, referring to FIG. 24B, the one or more suction ports 808 may be positioned radially inward of one or more native heart valve leaflets 804 during a deployment procedure for an implant. A distal end 814 of the capsule 806 may be positioned distal of the one or more suction ports 808.

The one or more suction ports 808 may apply the suction to draw the native heart valve leaflets 804 radially inward to contact the outer surface 810 of the elongate shaft 800. The suction from the one or more suction ports 808 may hold the native heart valve leaflets 804 in position against the outer surface 810 of the elongate shaft 800.

The elongate shaft 800 may be configured to at least partially deploy the implant from the implant retention area to the native heart valve 802 with the one or more suction ports 808 applying the suction to the native heart valve leaflets 804. For example, with the suction applied, the capsule 806 may be configured to be retracted to at least partially deploy the implant from the implant retention area.

The implant may comprise a prosthetic heart valve for deployment to the native valve. The implant may be configured similarly as examples of implants disclosed herein, including the implants shown in FIGS. 3A-3E for example. The implant, for example, may include one or more anchors that may be configured to hook over a distal tip of one or more of the native heart valve leaflets 804. FIG. 24C, for example, shows an implant 816 at least partially deployed with distal anchors 818 extending distally from the capsule 806. The distal anchors 818 may hook over the distal tips of the native heart valve leaflets 804.

The suction provided by the suction ports 808 may draw the distal tips of the native heart valve leaflets 804 radially inward, to improve the possibility of the distal anchors 818 hooking over the distal tips of the native heart valve leaflets 804. The position of the distal tips radially inward may reduce the possibility of a missed capture of the distal tips by an anchor 818 failing to hook over the distal tip and being positioned radially inward of the prosthetic valve leaflet 804. Such a configuration may cause the anchor 818 that missed capture of the leaflet to prop open the missed leaflet, and may result in paravalvular leakage. Thus, the suction provided by the suction ports 808 may reduce such possibility and improve the likelihood of capture of the leaflets 804 by the anchors 818.

The suction provided by the suction ports 808 may continue to be applied until the implant 816 is secured to the native heart valve 802. For example, the suction provided by the suction ports 808 may continue to be applied until the distal anchors 818 have hooked over the distal tips of the native heart valve leaflets 804. With the implant 816 secured, the suction provided by the suction ports 808 may be reduced and the implant 816 may be fully deployed to the implantation site. For example, FIG. 24D illustrates the implant 816 deployed to the native heart valve 802 with the suction ports 808 and the elongate shaft 800 being retracted from the implantation site.

The features of the examples of FIGS. 24A-24E may be utilized solely or in combination with any other example disclosed herein. Variations in the delivery system and the methods of utilizing the delivery system may be provided in examples.

Implants as disclosed herein may include one or more anchors that may be utilized to anchor the implant to the implantation site. For example, referring to the implant shown in FIG. 3E, an implant may include a valve body and a plurality of distal anchors 80 and a plurality of proximal anchors 82 for anchoring the implant to the implantation site. Referring to the implant shown in FIGS. 3A-3C, the implant may include a plurality of prosthetic valve leaflets 424, and the valve body 406 may support the plurality of prosthetic valve leaflets. The valve body 406 may include a valve frame with a distal portion and the plurality of distal anchors 430 may each have a proximal portion coupled to the distal portion of the valve frame 416.

Each of the distal anchors, for example, may each have a distal tip and may have a proximal portion configured to couple to a distal portion of the valve frame of the valve body. The implant may be configured to move from a crimped configuration, in which the distal anchors have an elongated shape to a deployed configuration in which the anchors extend radially outward from the valve body. The distal anchors may extend axially with the valve body in the crimped configuration. FIGS. 3A and 3E illustrate an implant in a deployed configuration.

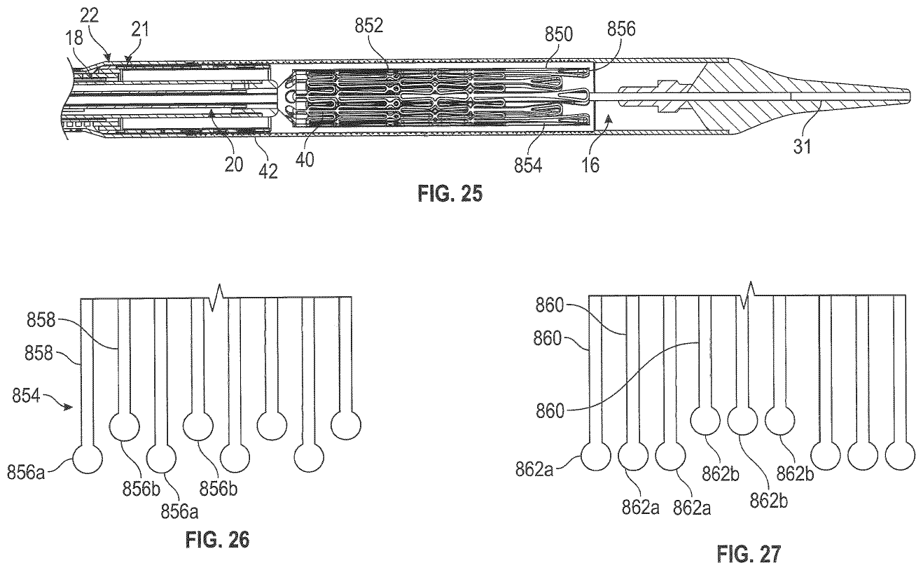

FIG. 25 illustrates an implant 850 in a crimped configuration and positioned within a deployment capsule of a delivery apparatus. The implant 850 may be configured similarly as the implants shown in FIGS. 3A and 3E, and may include a valve body 852 and a plurality of distal anchors 854. Each distal anchor 854 may include a distal tip 856.

At least one distal tip 856a in the crimped configuration may be offset longitudinally from a position of another distal tip 856b in the crimped configuration. Such a configuration may provide a variety of benefits including allowing for a smaller crimping profile or outer diameter due to the distal tips 856a, 856b having an offset position. Sequential deployment of the anchors may also result.

FIG. 26, for example, illustrates a flat profile of the distal anchors 854 of the implant 850 shown in FIG. 25. The distal tips 856a are shown to be offset longitudinally from the position of the distal tips 856b, with the distal tips 856a extending to a greater length that the distal tips 856b. As shown in FIG. 26, the distal tips 856a, b may each have a greater width than a shaft 858 that the respective distal tip 856a, b is coupled to. As such, an offset of the position of the distal tips 856a, b from each other may reduce the total diameter of the distal tips 856a, b when the implant 850 is crimped (as shown in FIG. 25 for example).

In examples, the distal anchors 854 may include a first plurality of distal anchors including distal tips 856a that are offset longitudinally from a second plurality of distal anchors including distal tips 856b. As shown in FIG. 26, for example, the first plurality of distal anchors may alternate circumferentially with the second plurality of distal anchors such that the distal tips 856a alternate circumferentially with the distal tips 856b. The distal tips 856a, b may alternate such that a short distal tip 856b is provided circumferentially adjacent to a long distal tip 856a, and the pattern repeats around the circumference of the crimped implant 850. Other configurations may be provided as desired.

FIG. 27, for example, illustrate a flat profile of distal anchors 860 in which a first plurality of distal anchors and the distal tips 862a are positioned adjacent to each other circumferentially and a second plurality of the distal anchors and distal tips 862b are positioned adjacent to each other circumferentially. The first plurality of distal tips 862a may be offset longitudinally from the position of the second plurality of distal tips 862b. In such a configuration, a reduced diameter of the distal tips 862a, 862b may be provided in a crimped configuration. Further, the short distal tips 862b may be positioned adjacent to each other such that in a deployed configuration the short distal tips 862b may have a reduced possibility of contact with a portion of a patient's anatomy. For example, a reduced possibility of contact with a ventricular wall may be provided, which may reduce a possibility of electrical conduction disturbance produced by the distal tips 862b. Further variations in the pattern of distal tips may be provided as desired.

In examples, the sequence in which the anchors may be deployed may be controlled with the length of the anchors. For example, in examples, longer anchors may be deployed initially and then shorter anchors may be deployed subsequently. In examples, a leaflet may be captured and then subsequently other anchors may be deployed to further capture that leaflet or another leaflet. Other forms of sequential deployment for the anchors may be utilized as desired.

In examples, the implant upon deployment may have a configuration similar to the configuration of the implant shown in FIGS. 3A and 3E, yet with the length of the distal anchors 854 being varied. The features of the implants shown in FIGS. 3A and 3E (e.g., prosthetic valve leaflets and sealing bodies) may be utilized with the implant. The distal anchors in a deployed configuration may be configured to have a hooked shape and may extend radially outward from the valve body. The distal anchors may be configured to hook over a distal tip of a leaflet of a native valve in a deployed configuration.

The features of the examples of FIGS. 25-27 may be utilized solely or in combination with any other example disclosed herein.

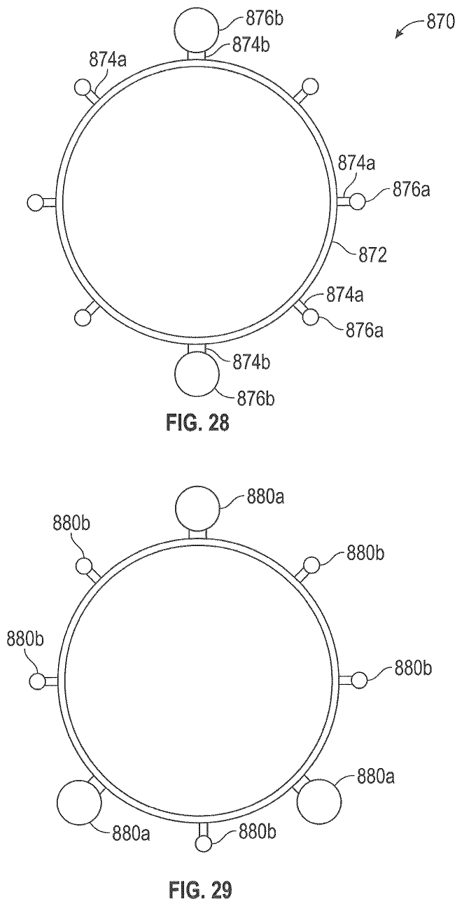

In examples, an implant may be provided that may include at least one distal tip configured to have a greater diameter than another of the distal tips. FIG. 28, for example, illustrates a top cross sectional view of an implant 870 including a valve body 872 and a plurality of distal anchors 874a, b coupled to the valve body 872. Each distal anchor 874a, b may have a respective distal tip 876a, b. The distal tips 876b may have a greater diameter than the distal tips 876a, as shown in FIG. 28 for example. The implant 870 may be otherwise configured similarly as the implants shown in FIGS. 3A and 3E, including a plurality of prosthetic valve leaflets and a valve body supporting the plurality of prosthetic valve leaflets. The distal anchors may be configured to hook over a distal tip of a leaflet of a native valve, with the distal tip of each of the plurality of distal anchors being positioned radially outward of the leaflet.

The distal tips 876b may be spaced circumferentially from the distal tips 876a, and may be spaced circumferentially from other of the larger diameter distal tips 876b. For example, as shown in FIG. 28, the larger diameter distal tips 876b may be positioned at opposite sides of the valve body 872 and along an axis extending through the center of the valve body 872. The smaller diameter distal tips 876a may be positioned circumferentially between the larger diameter distal tips 876b and circumferentially adjacent to each other. Various other configurations of distal tips may be provided as desired.

FIG. 29, for example, illustrates an example in which at least three larger diameter distal tips 880a may be provided that may be spaced circumferentially equidistant from each other. One or more smaller diameter distal tips 880b may be positioned circumferentially between the larger diameter distal tips 880a and may be positioned circumferentially adjacent to each other.

In examples, the larger diameter distal tips and the smaller diameter distal tips may be positioned to provide a desired anchoring at a portion of an implantation site. For example, a larger diameter distal tip may be provided at a portion of an implantation site that may require additional securement or fluid sealing, such as the commissures of native heart valve leaflets. A smaller diameter distal tip may be provided at a portion of an implantation site that may require reduced interference with portions of a patient's body, such as a heart wall. A configuration as shown in FIG. 28, for example, may be utilized with a mitral heart valve having two leaflets and accordingly two commissures. A configuration as shown in FIG. 29, for example, may be utilized with a tricuspid heart valve having two leaflets and accordingly three commissures. The larger diameter distal tips may be positioned at the commissures of the leaflets of the heart valves.

The larger diameter distal tips may have a greater diameter than the smaller diameter distal tips by being formed of material in a manner that produces a greater diameter. For example, the larger diameter distal tips may be formed from a solid material to have a greater diameter. In examples, the larger diameter distal tips may include a covering that increases the diameter of the distal tips. For example, a pad may be provided on the larger diameter distal tips that increases the diameter of the distal tips. The size of the covering or pad may be smaller with the smaller diameter distal tips.

In examples, the diameter of the larger diameter distal tips and/or the smaller diameter distal tips may be variable to adjust a size of the larger diameter distal tips and/or the smaller diameter distal tips. In examples, the variation in the diameter of the larger diameter distal tips and/or the smaller diameter distal tips may be provided by the larger diameter distal tips and/or the smaller diameter distal tips being inflatable.

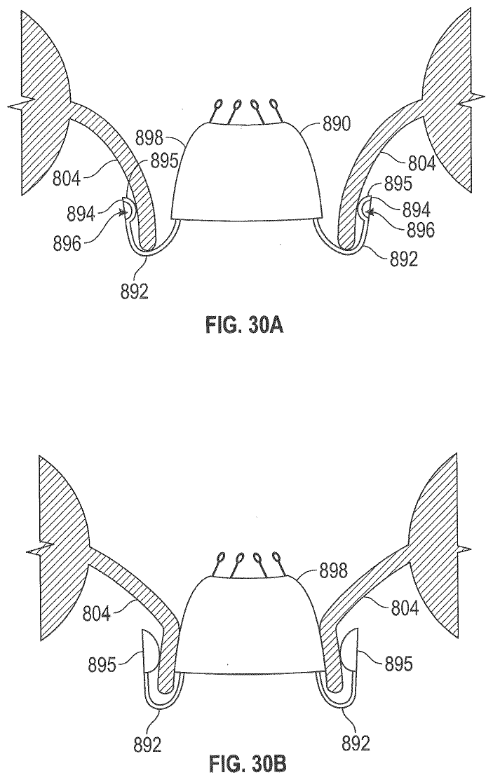

FIG. 30A, for example, illustrates a side cross sectional view of an implant in the form of a prosthetic heart valve 890 having distal anchors 892 including distal tips 894 that are configured to inflate. At least one of the distal tips 894 may be configured to be inflated to have a greater diameter than another of the distal tips 894. The distal tips 894, for example, may each include an inflatable body 895 that may be configured to be inflated with blood surrounding the distal tip 894 from the heart that the prosthetic heart valve 890 is deployed to. The blood may pass through an opening 896 in the respective inflatable body 895 that may cause the inflatable body 895 to inflate and have an increased diameter. FIG. 30B, for example, illustrates the inflatable bodies 895 having been inflated due to blood filling the inflatable bodies 895.

The inflation of the inflatable bodies 895 may improve the anchoring of the distal anchors 892 to the native heart valve. Further, improved sealing with the valve body 898 may result. The inflatable bodies 895 for example may inflate to press a portion of the heart valve, such as the native heart valve leaflets 804 against the valve body 898. The pressure applied by the inflatable bodies 895 of the leaflets 804 against the valve body 898 may improve a seal to reduce fluid flow around the valve body 898.

In examples, a portion of the distal anchors 892 may have inflatable distal tips, such that these inflatable distal tips have a greater diameter than other of the distal tips. For example, a first plurality of the plurality of distal anchors 892 may have distal tips that are configured to have a greater diameter than the distal tips of a second plurality of the distal anchors 892. In examples, all of the distal anchors may include inflatable distal tips that may inflate to a same diameter as desired.

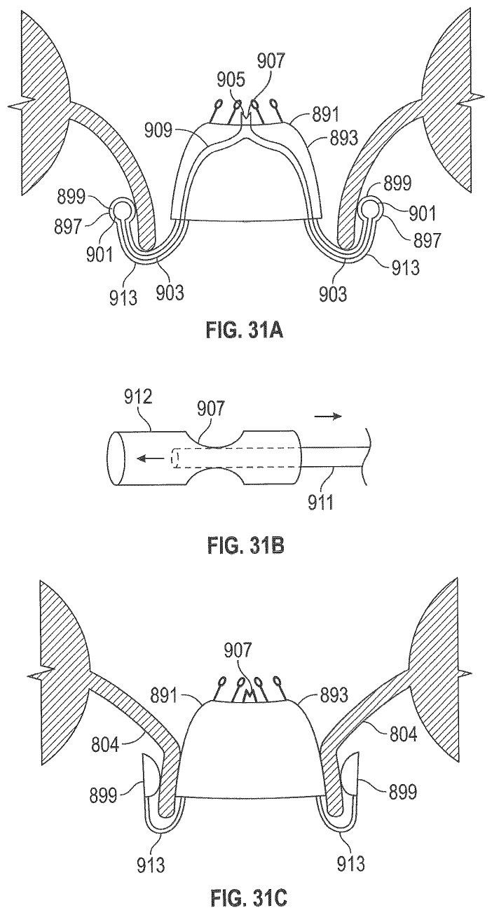

FIGS. 31A-31C illustrate an example of an implant in the form of a prosthetic heart valve 891 having distal anchors 913 including distal tips 897 that are configured to inflate. The distal tips 897, for example, may each include an inflatable body 899 having a bladder 901. The bladder 901 may be configured to be inflated with an inflation material to increase the size of the bladder 901. For example, one or more inflation conduits 903 may be provided that may have a distal end coupled to a respective bladder 901. The inflation conduits 903 may extend along a length of the distal anchors 913 to the respective bladder 901. The inflation conduits 903 may be for inflating a respective bladder 901 with the inflation material.

In examples, a proximal end 905 of the inflation conduits 903 may be coupled to the prosthetic heart valve 891 and may include a valve 907 that may separably couple to a tube for passing the inflation material into the inflation conduit 903. In examples, the inflation conduit 903 may include a manifold 909 for distributing the inflation material to a plurality of the inflation conduits 903. As such, fluid passed through the valve 907 may be distributed to a plurality of the inflatable bodies 899 and the distal anchors 913 via the manifold 909. Other configurations of inflatable bodies and inflation conduits may be utilized as desired.

FIG. 31B illustrates a close up view of a valve 907 that may be utilized to inflate the inflatable bodies 899. The valve 907 may comprise a pinch valve, or another form of valve that may engage with a tube 911 for passing the inflation material into the inflation conduit 903. The valve 907 may be configured such that upon inflation of the inflatable bodies 899, the tube 911 may be retracted from the valve 907 with the valve closing automatically. In examples, other forms of valves, such as a check valve, may be utilized as desired. The tube 911 may be part of a delivery system for the inflatable bodies 899 and may be withdrawn following inflation of the inflatable bodies 899.

The inflatable bodies 899 may inflated to a desired amount and may press the leaflets 804 of the heart valve against the valve body 893 of the prosthetic heart valve 891. FIG. 31C, for example, illustrates the inflatable bodies 899 having been inflated. The inflatable bodies 899 may press the leaflets 804 of the heart valve against the valve body 893 to improve sealing with the valve body 893.

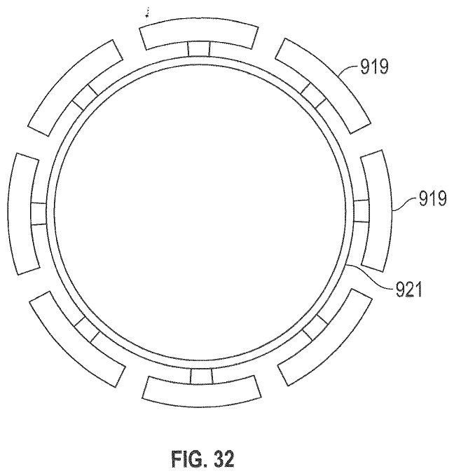

In examples, the inflatable bodies may be shaped as desired to enhance sealing with the native heart valve. FIG. 32, for example, illustrates a top cross sectional view of a plurality of inflatable bodies 919, each having a flattened arcuate shape. For example, the shape may comprise a semi-cylindrical shape that extends around the valve body 921. In such a configuration, the plurality of inflatable bodies 919 may form a ring around the native heart valve leaflets to press the native heart valve leaflets against the valve body 921. Such a configuration may enhance sealing with the native heart valve.

In examples, a portion of the distal tips of the distal anchors may include inflatable bodies configured to inflate and a portion of the distal tips may be non-inflatable. The inflatable bodies accordingly may have a greater diameter then the non-inflatable distal tips. In examples, each of the distal tips of the distal anchors may be inflatable as desired.

An inflation material that may be utilized in examples herein may comprise a fluid such as saline or other forms of fluid. The inflation material may be a liquid, foam, epoxy, gas, or other material. The inflation material may be liquid to result in a hydraulic inflation of the bodies disclosed herein. An inflation material in the form of a gas may comprise carbon dioxide or helium, among other forms of gas.

In examples, the inflation material may comprise a hardenable material. The inflation material may be configured to harden over time to enhance the sealing of the sealing body. The hardenable material that may be introduced into an inflatable body at a first, relatively low viscosity and converted to a second, relatively high viscosity. Viscosity enhancement may be accomplished through a variety of UV initiated or catalyst initiated polymerization reactions, or other chemical system. The end point of the viscosity enhancing process may result in a hardness anywhere from a gel to a rigid structure, depending on the desired performance.

A hardenable material may comprise an epoxy. The epoxy may be hardened by mixing materials that harden when combined. The hardening catalyst may be delivered during implantation or later. The hardenable material may be biocompatible and able to conform to the shape of the local native valve. In examples, the hardenable material may be bioresorbable.

In examples, the inflation material may be radiopaque for visualization during implantation. A radiopaque material may be added during filling, as part of a hardening process for example. In examples, a portion of a sealing body, such as a sealing skirt may be radiopaque to allow for visualization during implantation.

In examples, the inflation material may comprise a gel or a foam, which may be biocompatible, and may be configured to harden over time. A gel or foam may be inserted into the sealing body, or may be provided in capsules that dissolve upon implantation to allow for expansion.

In examples, a gel may be utilized that may be made via polymer precipitation from biocompatible solvents. Various siloxanes may be utilized as inflation gels as well. Other gel systems that may be utilized may include phase change systems that gel upon heating or cooling from their initial liquid or thixotropic state. Gels may also comprise thixotropic material that undergo sufficient shear-thinning so that they may be readily injected through a fluid conduit yet are also gel-like at zero or low shear rates.

In examples, an inflation material may contain a foaming agent. The foaming agent may generate pressure within the inflatable body.

Any of the inflation materials disclosed herein may be biocompatible in examples and may be bioresorbable if desired. A bioresorbable sealing body may improve sealing through tissue adhesion with the native valve.

The features of the examples of FIGS. 28-32 may be utilized solely or in combination with any other example disclosed herein.

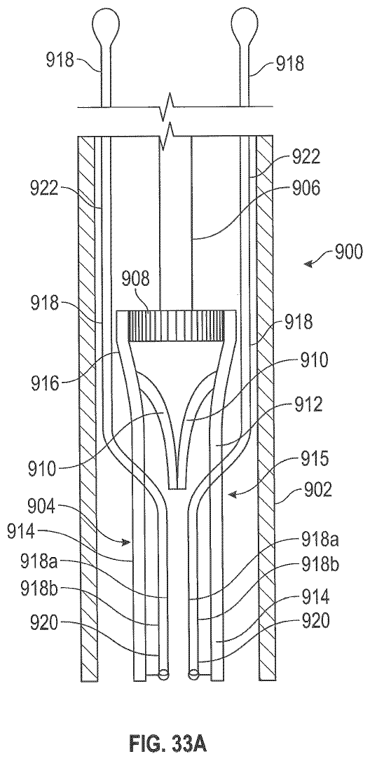

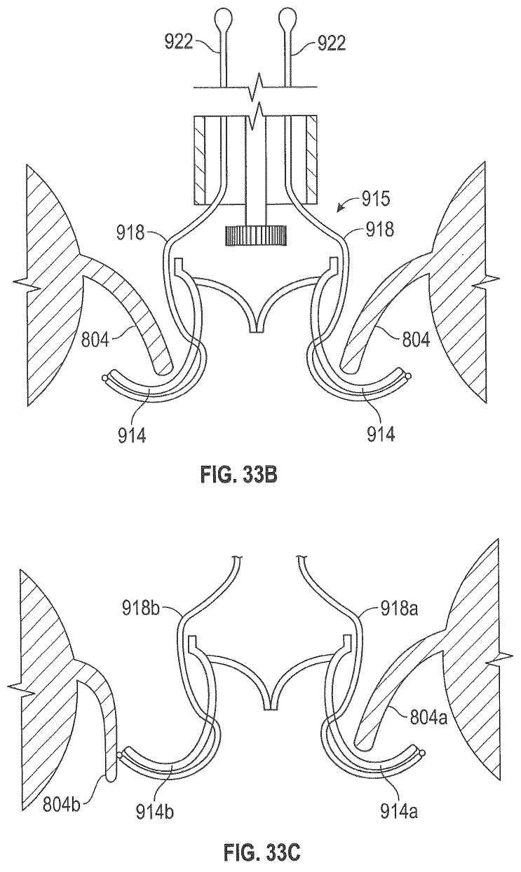

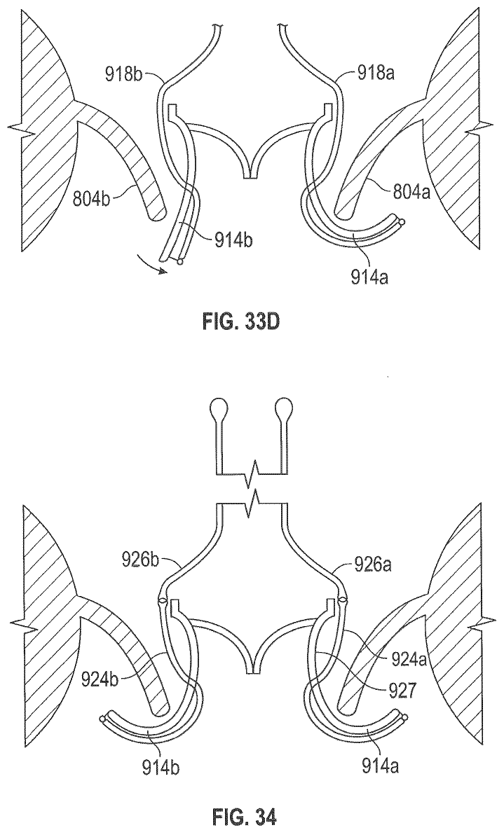

Delivery systems that may be utilized herein may include one or more sutures that may be configured to couple to the implant and apply a compressive force to the implant radially inward. The one or more sutures may be for adjusting a position of at least one distal anchor of the implant. FIG. 33A, for example, illustrates an example of an elongate shaft 900 of a delivery system including a capsule 902 surrounding an implant retention area 904 for retaining an implant. The capsule 902 may surround an inner shaft 906 that may include a coupler 908 that may be configured to couple to a portion of the implant, such as a proximal end of the implant 915. The coupler 908 may be configured to couple to end couplers of the implant 915, similar to the operation of the inner retention member or inner retention ring 40 shown in FIGS. 2A and 2B for example.

The implant 915, in examples, may be configured similarly as the implants shown in FIGS. 3A and 3E. For example, the implant 915 may include a plurality of prosthetic valve leaflets 910 and may include a valve body 912 supporting the plurality of prosthetic valve leaflets 910. A plurality of distal anchors 914 may be coupled to the valve body 912. The valve body 912 may include a valve frame 916 that may be coupled to the plurality of distal anchors 914. The implant 915 is shown in a crimped configuration in FIG. 33A, with a proximal end coupled to the coupler 908.

The one or more sutures 918 may be configured to couple to the implant 915 and apply a compressive force to the implant 915 radially inward. The compressive force may move an anchor radially inward. The one or more sutures 918, for example, may include a first portion 920 that is configured to couple to the implant 915. The first portion 920 may be configured to couple to the body of the implant 915. The sutures 918 as shown in FIG. 33A may be configured to couple to distal anchors 914 of the implant 915. The sutures 918 may be configured to couple to at least one of the plurality of distal anchors 914 and apply a compressive force radially inward. Each suture 918, for example, may include a loop formed by a first suture length 918a and a second suture length 918b, with the loop passing through a portion of a respective distal anchor 914. Each distal anchor 914, for example, may include an aperture or other structure for the loop of a suture 918 to pass through.

In examples, each suture 918 may pass from an interior of the implant 915 to a position radially outward of the implant 915. For example, each suture 918 may include a second portion 922 extending proximally from the first portion 920, with the second portion 922 configured to pass through the valve frame 916 to a position radially outward of the plurality of prosthetic valve leaflets 910. In such a configuration, the possibility of contact between the suture 918 and the prosthetic valve leaflets 910 may be reduced, to reduce the possibility of the suture 918 interfering with the operation of the prosthetic valve leaflets 910. The second portion 922 of the sutures 918 may extend proximally from the distal end of the valve frame 916 to the elongate shaft 900. In examples, the sutures 918 may pass directly through the flow channel of the implant from the distal anchors to the distal end of the implant through the flow channel.

The second portion 922 of the sutures may extend proximally from the distal end of the body of the implant 915 to the elongate shaft 900. The second portion 922 of the sutures may extend proximally through the elongate shaft 900 for access at a proximal portion of the elongate shaft 900. For example, the second portion 922 of the sutures 918 may be accessible at a proximal portion of the elongate shaft 900 for control by a user. A user may be able to control the sutures 918 and retract or release the sutures 918 to control the compressive force applied by the sutures 918 against the distal anchors 914.

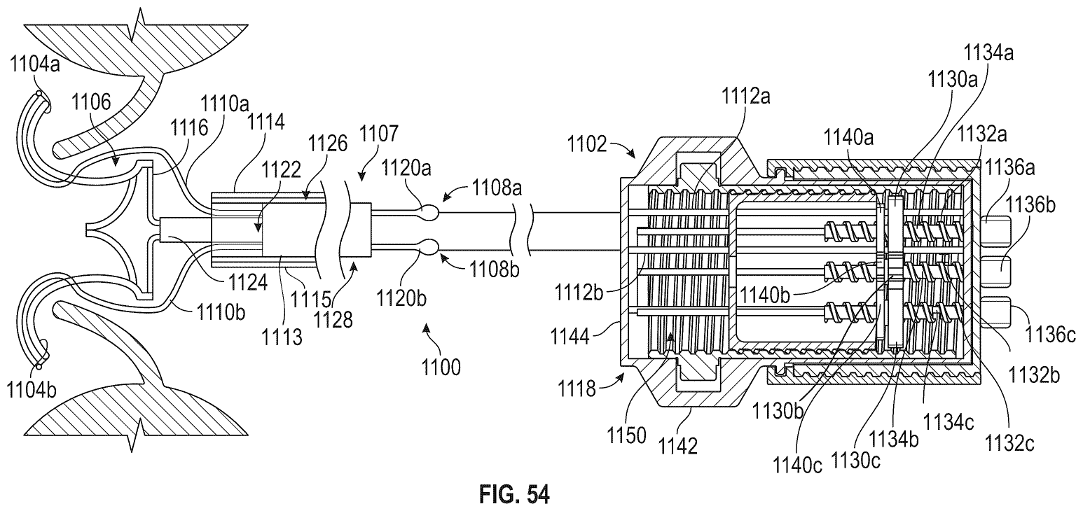

In examples, a control mechanism may be utilized to control a movement or deflection of the anchors. For example, features of a control mechanism as shown in FIGS. 54-63 may be utilized as desired. In examples, one or more actuators may be provided at a proximal portion of a delivery apparatus. A handle of a delivery apparatus, for example, may utilize one or more actuators (e.g., control knobs, or other forms of actuators) for allowing a user to actuate movement of the sutures 918. The one or more actuators, for example, may be coupled to the second portion 922 of the sutures 918. The handle may have a configuration as shown in FIG. 10 or may have another configuration as desired. Other forms of control may be utilized in examples as desired.

A routing of the sutures 918 from an interior of the implant 915 to an exterior of the implant 915 may allow for a proximal force upon the sutures 918 to apply a radially inward compressive force against the implant 915. The radially inward force, in examples, may control deployment of the implant and may control a radial position of the distal anchors 914. For example, retraction of the sutures 918 may compress the distal anchors 914 radially inward and advancement of the sutures 918 may move the distal anchors 914 radially outward. Such movement may be utilized for individual anchor actuation, control of the flow channel size, and/or recapture of the implant as desired.

FIG. 33B, for example, illustrates a deployment of an implant 915 to a native heart valve. The distal anchors 914 may be anchored to heart valve leaflets 804 by the sutures 918 being released, to allow the distal anchors 914 to expand radially outward. The distal anchors 914 may hook over the distal tips of the prosthetic valve leaflets 804.

The distal anchors 914 may be configured to be retracted by the one or more sutures 918 being retracted. For example, the second portion 922 may be retracted proximally to apply a proximal force to the sutures 918, causing one or more sutures 918 to apply the compressive force to the implant 915 radially inward and cause the distal anchors 914 to be drawn radially inward. The second portion 922 may be retracted proximally to cause the sutures 918 to adjust the position of the anchors 914. Such a feature may be utilized for a missed capture of a leaflet or a desired recapture of a leaflet.

FIG. 33C, for example, illustrates an example in which the implant 915 is deployed with an anchor 914a capturing a leaflet 804a and an anchor 914b missing capture of a leaflet 804b. The anchor 914b may prop open the position of the leaflet 804b, which may undesirably lead to leakage around the valve body. The suture 918b may be retracted. Retraction of the suture 918b may move the distal anchor 914b radially inward.

FIG. 33D illustrates the anchor 914b having been retracted, which may allow the heart valve leaflet 804b to move radially inward. The anchor 914b may be moved from a hooked configuration towards an elongated configuration. The suture 918b may then be released, to allow the distal anchor 914b to extend radially outward and hook over the distal tip of the heart valve leaflet 804b and anchor to the leaflet, in a similar configuration as shown in FIG. 33B. In examples, the suture 918b may be retracted independent of the suture 918a. One or more of the distal anchors may be operated independently via control of the one or more sutures. Each suture may move a distal anchor of the implant radially inward independent of another distal anchor of the implant.

In examples, each suture 918a, 918b may be configured as a continuous loop that may extend from the implant to a proximal portion of the delivery apparatus. Control of the sutures 918a, 918b may occur at the proximal portion of the delivery apparatus through tension and release of the proximal portion of the sutures. The looped configuration of the sutures 918a, b may allow each suture 918a, b to be cut and withdrawn through the delivery apparatus to be released from the implant. Other configurations of sutures may be utilized in examples.

FIG. 34 illustrates a configuration of sutures in which a plurality of sutures may be utilized to control each distal anchor 914a, b of an implant 927. A first suture loop 924a may couple to the distal anchor 914a and may pass through the valve body. A second suture loop 926a may be coupled to the first suture loop 924a, and the second suture loop 926a may extend proximally through the elongate shaft of the delivery apparatus. The first suture loop 924b and the second suture loop 926b may be configured similarly as the first suture loop 924a and the second suture loop 926a.

Upon the implant 927 being deployed, the second suture loops 926a, b may be cut and withdrawn from the first suture loops 924a, b. The first suture loops 924a, b may remain in position upon deployment.

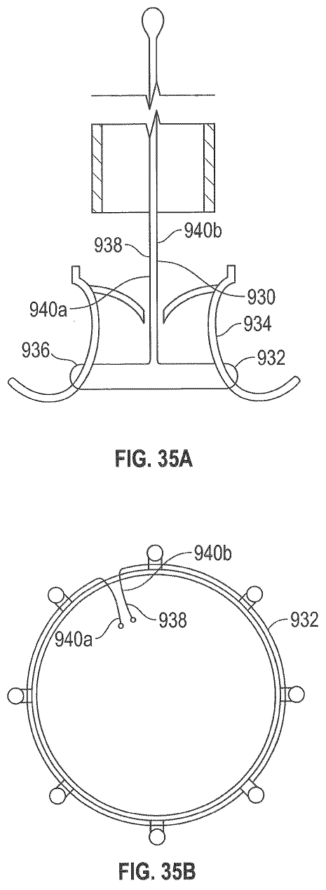

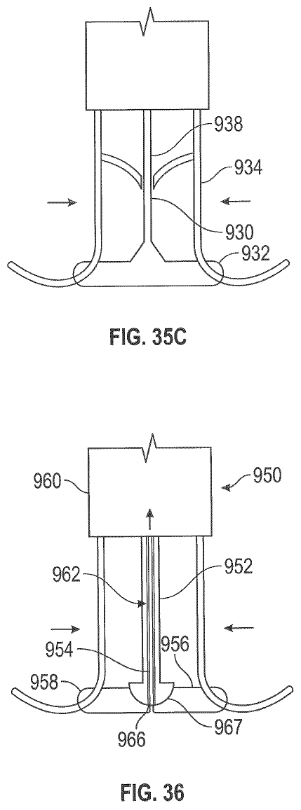

FIG. 35A illustrates an example in which the one or more sutures 930 form a loop 932 extending circumferentially about the implant 934 and configured to apply a compressive force to the implant radially inward. The one or more sutures 930, for example, may extend within the interior of the implant 934 and may then pass radially outward of the implant 934 to form the loop 932. A first portion 936 of the one or more sutures 930 may form the loop 932 and may extend exterior of the implant 934. A second portion 938 may extend interior of the implant 934 and may extend proximally through the delivery apparatus. The second portion 938 may include a first suture length 940a coupling to a first end of the loop 932 and a second suture length 940b coupling to a second end of the loop 932.

FIG. 35B, for example, illustrates a top cross sectional view of the implant 934. The first suture length 940a and second suture length 940b are shown to pass radially outward from a portion of the valve body to form the loop 932 around the valve body. Referring to FIG. 35A, as the second portion 938 of the one or more sutures 930 is retracted, the loop 932 may be drawn radially inward to compress the implant 934.

FIG. 35C illustrates the second portion 938 of the one or more sutures 930 having been retracted and the loop 932 applying a compressive force to draw the implant 934 radially inward. Such a feature may control deployment of the implant 934 and allow the distal anchors to be recaptured or repositioned if desired.

In examples, a control mechanism may be utilized to control a movement or deflection of the anchors. For example, features of a control mechanism as shown in FIGS. 54-63 may be utilized as desired. In examples, one or more actuators may be provided at a proximal portion of a delivery apparatus. A handle of a delivery apparatus, for example, may utilize one or more actuators (e.g., control knobs, or other forms of actuators) for allowing a user to actuate movement of the sutures 930. The one or more actuators, for example, may be coupled to the second portion 938 of the sutures 930. The handle may have a configuration as shown in FIG. 10 or may have another configuration as desired. Other forms of control may be utilized in examples as desired.

The loop 932 may extend around the valve body in examples, or may extend circumferentially around one or more of the distal anchors in examples. Other configurations of sutures may be utilized in examples.

FIG. 36, for example, illustrates an example in which the elongate shaft 950 includes an inner shaft 952 that the second portion 954 of a suture 956 forming a loop 958 may pass through. The inner shaft 952 may extend within a capsule 960 of the elongate shaft 950, for example. The inner shaft 952 may pass through the implant (e.g., through the flow channel) and may have a tip positioned at a central portion of the implant, or a distal portion of the implant, or other position relative to the implant. The inner shaft 952 may be configured to engage the second portion 954 of the one or more sutures 956. The inner shaft 952 may include an interior lumen 962 that the second portion 954 of the suture 956 may pass proximally through.

The second portion 954 of the suture 956 may be configured to be retracted proximally through the inner shaft 952 to cause the suture 956 to apply the compressive force to the implant radially inward. The second portion 954 may be retracted to cause the sutures 956 to adjust the position of the anchors.

In examples, a control mechanism may be utilized to control a movement or deflection of the anchors. For example, features of a control mechanism as shown in FIGS. 54-63 may be utilized as desired. In examples, one or more actuators may be provided at a proximal portion of a delivery apparatus. A handle of a delivery apparatus, for example, may utilize one or more actuators (e.g., control knobs, or other forms of actuators) for allowing a user to actuate movement of the suture 956. The one or more actuators, for example, may be coupled to the second portion 954 of the suture 956. The handle may have a configuration as shown in FIG. 10 or may have another configuration as desired. Other forms of control may be utilized in examples as desired.

In examples, the distal end 967 of the inner shaft 952 may include an opening 966 for the interior lumen 962. The opening 966 may be positioned in a plane with the loop 958 of the suture 956, or may have another location as desired. The opening 966 may be positioned to receive the suture 956, which may pass through the interior lumen 962.

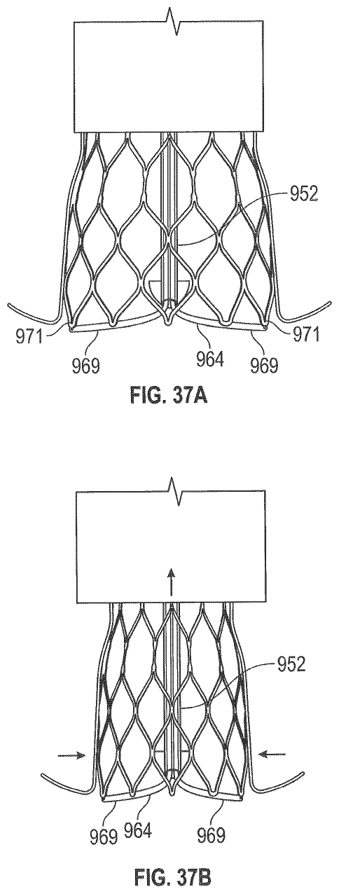

In examples, an inner shaft 952 may be utilized with one or more sutures 964 extending radially outward from the inner shaft 952, to couple to the valve frame. FIG. 37A, for example, illustrates that one or more sutures 964 may extend radially outward from the inner shaft 952 to couple to the valve frame. Distal ends 969 of the sutures 964 may couple to the valve frame. For example, the valve frame may include distal apices 971 that the distal ends 969 of the sutures 964 may couple to. The sutures 964 may be configured to be withdrawn radially inward to compress the valve frame radially inward.

FIG. 37B, for example, illustrates the sutures 964 having been retracted radially inward and into the inner shaft 952, to radially compress the implant.

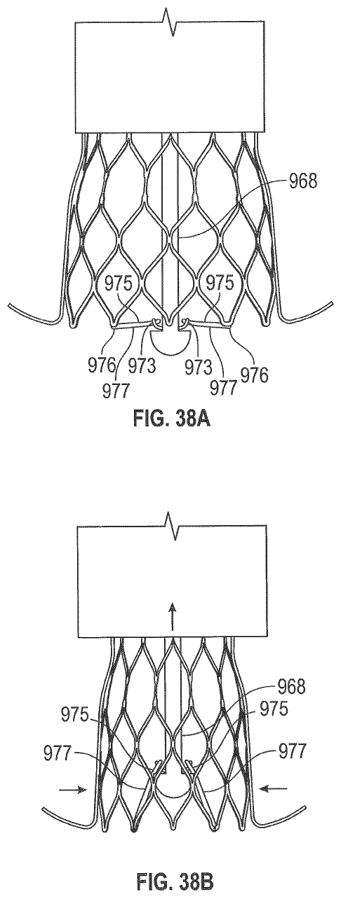

FIGS. 38A-B illustrate an example in which an inner shaft 968 may be configured to couple to one or more sutures 977 such that retraction of the inner shaft 968 causes the one or more sutures 977 to apply the compressive force to the implant radially inward. The inner shaft 968 may be retracted to cause the sutures 977 to adjust the position of the anchors. FIG. 38A, for example, illustrates an inner shaft 968 having one or more couplers 973 that are configured to engage second portions 975 of sutures 977. The sutures 977 may include first portions 976 that may couple to the valve frame. The couplers 973 of the inner shaft 968 may be configured to hook or otherwise engage the second portions 975 of the sutures 977 such that proximal movement of the inner shaft 968 causes the one or more sutures 977 to apply the compressive force to the implant radially inward.

FIG. 38B, for example, illustrates the inner shaft 968 having been retracted, with the couplers 973 having been drawn proximally and thus radially inward. The implant may be compressed radially inward due to the movement of the inner shaft 968.

Upon the implant being deployed to a desired amount, the inner shaft 968 may be advanced distally, which may release the sutures 977 from the inner shaft 968. The second portions 975 of the sutures 977 may release from the inner shaft 968, thus allowing the inner shaft 968 to be separated from the sutures 977 and the implant. Various other configurations of coupling between sutures and an inner shaft may be provided.

In examples, a control mechanism may be utilized to control a movement or deflection of the inner shaft 968 and accordingly the anchors. In examples, one or more actuators may be provided at a proximal portion of a delivery apparatus. A handle of a delivery apparatus, for example, may utilize one or more actuators (e.g., control knobs, or other forms of actuators) for allowing a user to actuate movement of the inner shaft 968. The one or more actuators, for example, may be coupled to a proximal portion of the inner shaft 968. The handle may have a configuration as shown in FIG. 10 or may have another configuration as desired. Other forms of control may be utilized in examples as desired.

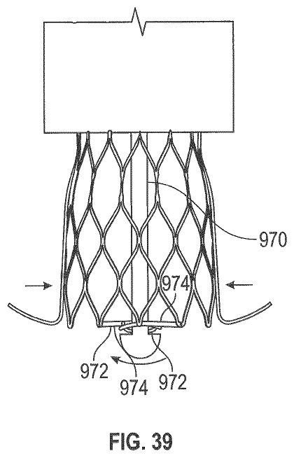

FIG. 39, for example, illustrates an example in which an inner shaft 970 may be rotated to cause one or more sutures 972 to apply the compressive force to the implant radially inward. The inner shaft 970 may be rotated to cause the sutures 972 to adjust the position of the anchors. The one or more sutures 972 may each include a second portion 974 that may couple to the inner shaft 970. The inner shaft 970 may include couplers for engaging the second portions 974 of the one or more sutures 972. The second portion 974 may couple to the inner shaft 970 such that rotation of the inner shaft 970 causes the sutures 972 to apply the compressive force to the implant radially inward. The inner shaft 970 may be rotated to cause the second portion 974 of the one or more sutures 972 to wrap around the inner shaft 970 when the inner shaft 970 rotates, thus applying a compressive force to the implant radially inward. The inner shaft 970 may be rotated in an opposite direction to allow the one or more sutures 972 to expand radially outward and thus allow the implant to expand radially outward.

Upon the implant being deployed to a desired amount, the inner shaft 970 may be released from the one or more sutures 972. For example, the inner shaft 970 may be advanced distally, which may release the sutures 972 from the inner shaft 970 in a similar manner as the inner shaft 968. In examples, a combination of retraction or rotation, or solely retraction or solely rotation may be utilized to cause the one or more sutures to apply the compressive force to the anchors. Retraction or rotation may be utilized in any example disclosed herein, including an example having a loop coupling to the implant.

In examples, a control mechanism may be utilized to control a movement or deflection of the inner shaft 970 and accordingly the anchors. In examples, one or more actuators may be provided at a proximal portion of a delivery apparatus. A handle of a delivery apparatus, for example, may utilize one or more actuators (e.g., control knobs, or other forms of actuators) for allowing a user to actuate movement of the inner shaft 970. The one or more actuators, for example, may be coupled to a proximal portion of the inner shaft 970. The handle may have a configuration as shown in FIG. 10 or may have another configuration as desired. Other forms of control may be utilized in examples as desired.

The radially inward compressive force discussed in regard to FIGS. 33A-39 may be utilized for anchor actuation, inflow expansion, implant release, and/or implant recapture. The radially inward compressive force, for example, may control an implant release by controlling a rate of expansion of an implant, or by controlling a rate of deployment of anchors, among other features. The features of the examples of FIGS. 33A-39 may be utilized solely or in combination with any other example disclosed herein.

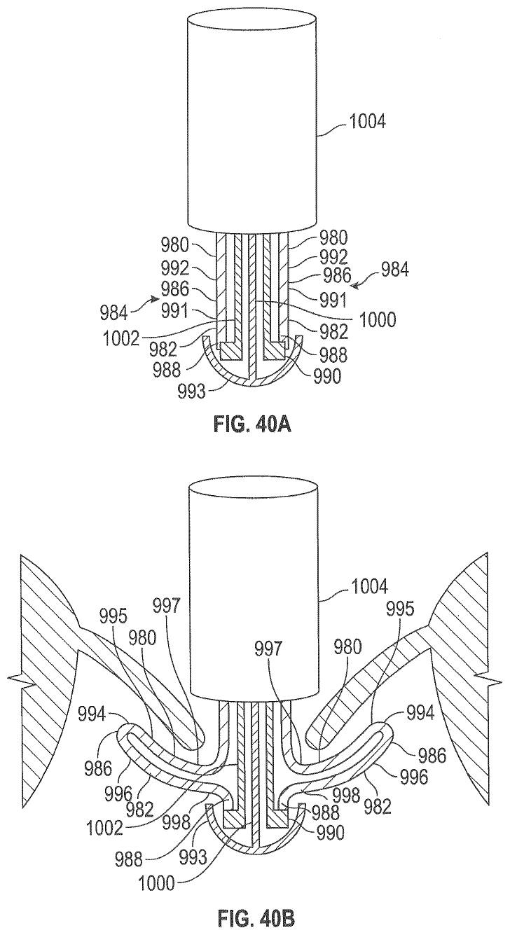

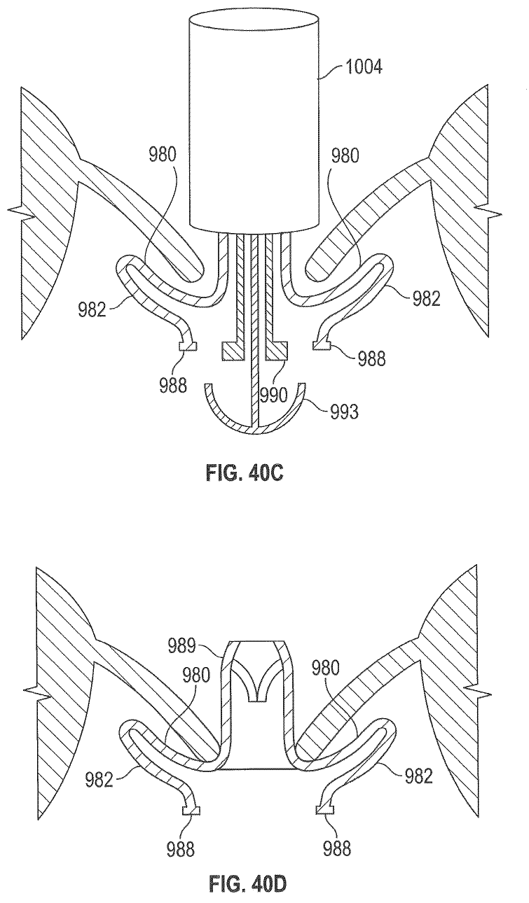

FIGS. 40A-45C illustrate examples of prosthetic valves including one or more anchors each configured to anchor the prosthetic valve to a native valve and each including a first arm and a second arm and configured to extend radially outward to a tip, the tip including a loop coupling the first arm to the second arm and the first arm configured to be moved relative to the second arm to vary a distance between the first arm and the second arm.

FIG. 40A, for example, illustrates a prosthetic valve in a stretched configuration in which a first arm 980 is moved away from the second arm 982. The first arm 980 and second arm 982 may be elongated with the first arm 980 positioned proximal of the second arm 982. A portion 986 of the anchors 984 that is positioned between the first arm 980 and the second arm 982 may form a tip of the respective anchor 984. A loop (shown in FIG. 40B) of the tip may be straightened in FIG. 40A.

The second arm 982 may include a distal end 988 that may be configured to couple to a coupler 990 of a delivery system. The coupler 990, for example, may be configured to couple to tabs at the distal end 988 of the second arm 982. A retaining body 993 may extend over the distal end 988 and retain the distal end 988 of the second arm 982 to the coupler 990. The distal end 988 of the second arm 982 may be configured to expand radially outward when released from the coupler 990.

The second arm 982 may be configured to extend proximally to a proximal portion 991 that may couple to the portion 986 forming the tip in the deployed configuration. The second arm 982 may have an elongate shape from the distal end 988 to the proximal portion 991.

The first arm 980 may have a distal portion 992 that may couple to the portion 986 forming the tip in the deployed configuration. The first arm 980 may extend proximally from the distal portion 992 to couple to a valve body 989 for the implant, as shown in FIG. 40D, for example.

The first arm 980 and the second arm 982 may be flexible and may be configured to move from the stretched configuration to a deployed configuration. The portion 986 forming the tip may be flexible and the first arm 980 and the second arm 982 may be configured to pivot about the portion 986 forming the tip upon movement from the stretched configuration to the deployed configuration.

FIG. 40B, for example, illustrates the anchors having moved from the stretched configuration to the deployed configuration. The first arm 980 and the second arm 982 have moved towards each other. The portion 986 is shown to form a tip 994 of a respective anchor, with the tip 994 including a loop that couples the first arm 980 to the second arm 982. The first arm 980 and the second arm 982 each extend radially inward from the tip 994.

The first arm 980 may be positioned proximal of the second arm 982. The first arm 980 may be positioned radially inward of the second arm 982. In examples, the first arm 980 may extend parallel with the second arm 982.

The first arm 980 may include a radially outward portion 995 that may couple to the loop of the tip 994 of the respective anchor. The first arm 980 may include a radially inward portion 997 that may couple to the valve body.

The second arm 982 may include a radially outward portion 996 that may be coupled to the loop of the tip 944. The second arm 982 may include a radially inward portion 998 that may include the distal end 988 of the second arm 982. The distal end 988 of the second arm 982 may comprise a free end of the second arm 982 that may be coupled to the coupler 990 of the delivery apparatus in FIG. 40B.

The first arm 980 and second arm 982 may have a hooked shape that may allow the anchor to hook around a distal tip of the prosthetic valve leaflet. The first arm 980 and the second arm 982 may comprise a distal anchor configured to hook over a distal tip of a leaflet of the native valve. The first arm 980 and the second arm 982 may extend parallel with each other from the respective radially inward portions 997, 998 of the arms 980, 982 to the respective radially outward portions 995, 996 of the arms 980, 982.

In examples, movement of the anchors from the stretched configuration to the deployed configuration may be controlled with operation of a delivery system. The delivery system, for example, may include the coupler 990 and the retaining body 993. The coupler 990 may be positioned at a distal portion of an inner shaft 1000 that may extend interior of the implant. The retaining body 993 may be positioned at a distal portion of a sheath 1002 and may extend over the distal ends 988 of the implant. The retaining body 993 may hold the distal ends 988 of the implant to the coupler 990 and thus preventing the distal ends 988 from releasing from the coupler 990.

In examples, a control mechanism may be utilized to control a movement or deflection of the anchors. In examples, one or more actuators may be provided at a proximal portion of a delivery apparatus. A handle of a delivery apparatus, for example, may utilize one or more actuators (e.g., control knobs, or other forms of actuators) for allowing a user to actuate movement of the coupler 990 and the retaining body 993. The one or more actuators, for example, may be coupled to a proximal portion of the inner shaft 1000 and sheath 1002. The handle may have a configuration as shown in FIG. 10 or may have another configuration as desired. Other forms of control may be utilized in examples as desired.

The delivery system may be configured such that the inner shaft 1000 may be slid proximally and distally along with the sheath 1002 to maintain a position of the retaining body 993 relative to the coupler 990. The inner shaft 1000 and sheath 1002 may be slid proximally together to axially compress the distal anchors and move the distal anchors from the stretched configuration to the deployed configuration. The inner shaft 1000 and the sheath 1002 may be retracted proximally together to a desired amount to allow the distal anchors to move to the deployed configuration and expand radially outward to a desired amount.

The inner shaft 1000 and the sheath 1002 may be advanced distally together to retract the distal anchors prior to release of the distal anchors from the coupler 990. As such, in the event of a missed capture of a heart valve leaflet the distal anchors may be retracted in an attempt to recapture the heart valve leaflets upon the distal anchors being expanded again. With the distal anchors in a desired position, the retaining body 993 may be advanced relative to the coupler 990 to release the distal end 988 of the implant from the coupler 990.

FIG. 40C, for example, illustrates the retaining body 993 having been advanced relative to the coupler 990. The retaining body 993 may uncover the distal ends 988 of the second arms 982 to release the distal ends 988 from the coupler 990. The distal anchors may hook around the prosthetic heart valve leaflets to anchor the implant to the heart valve. The first arm 980 may have a hooked shape in the deployed configuration and the second arm 982 may have a hooked shape in the deployed configuration.

With the distal anchors deployed, a capsule 1004 surrounding the implant may be retracted to deploy the implant. FIG. 40D, for example, illustrates the capsule 1004 retracted. The implant is deployed to the implantation site with the distal anchors hooking around the prosthetic valve leaflets. The first arms 980 may be integral with the valve body 989.

The implant may be configured similarly as examples of implants disclosed herein, such as the implants shown in FIGS. 3A-3E, and may include a plurality of prosthetic valve leaflets and a valve body 989 supporting the plurality of prosthetic valve leaflets.

In examples, the valve body may include an inner body and an outer body. FIG. 41, for example, illustrates an example in which the radially inward portion of the first arms 1006 are integral with an inner body 1008. An outer body 1010 may be provided that may be coupled to the inner body 1008. The outer body 1010 may comprise a sealing body that may be configured to form a seal with a portion of a native heart valve. In examples, the outer body 1010 may comprise an outer frame and the inner body 1008 may comprise an inner frame of the valve body. The outer frame may be positioned radially outward of the inner frame.

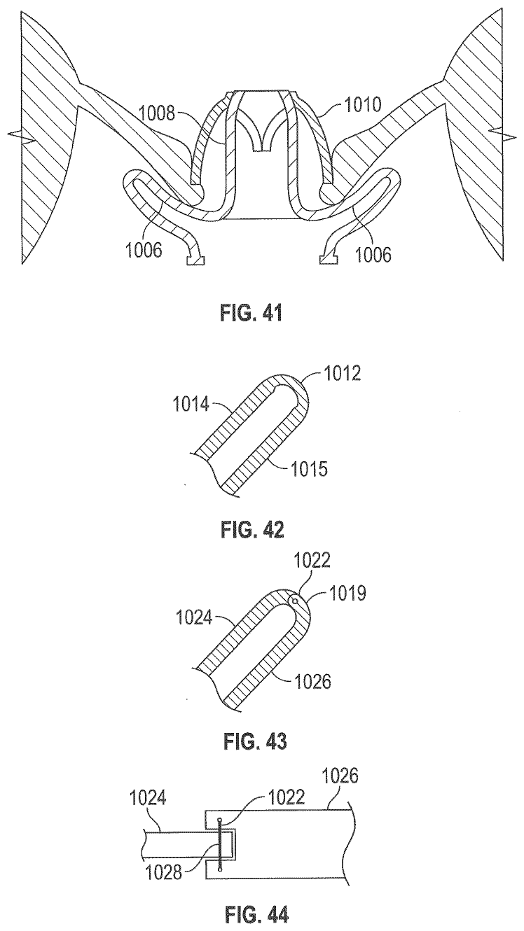

FIG. 42 illustrates an example of a tip 1012 of a distal anchor that may be utilized in examples herein. The tip 1012 may comprise a section positioned between the first arm 1014 and the second arm 1015 that is thinner than the first arm 1014 and/or the second arm 1015. The thinness of the tip 1012 may allow for flexibility of the distal anchor at the tip 1012.

FIG. 43 illustrates an example of a tip 1019 including a hinge 1022 that forms the loop and that couples the first arm 1024 to the second arm 1026. FIG. 44 illustrates a top view of the hinge 1022. The hinge 1022 may include a pivot 1028 that may pass through the first arm 1024 and may couple the first arm 1024 to the second arm 1026. The hinge 1022 may be configured to allow the first arm 1024 to rotate relative to the second arm 1026.

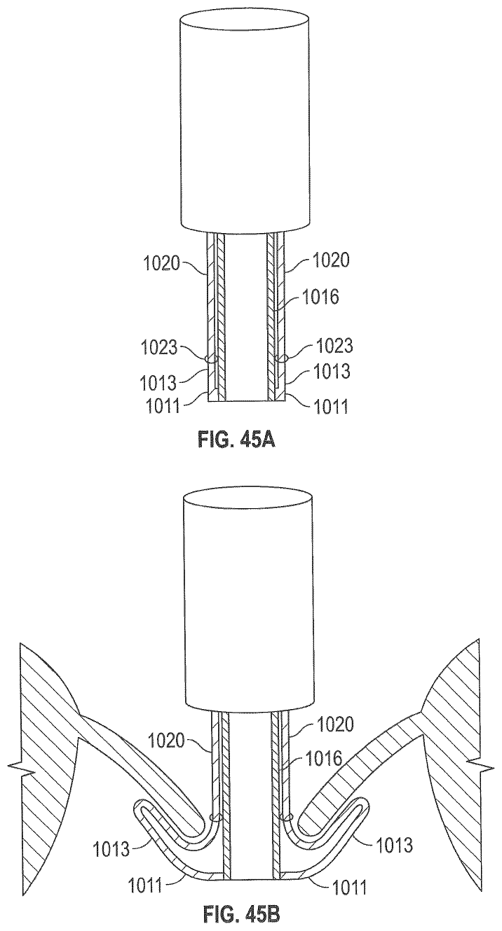

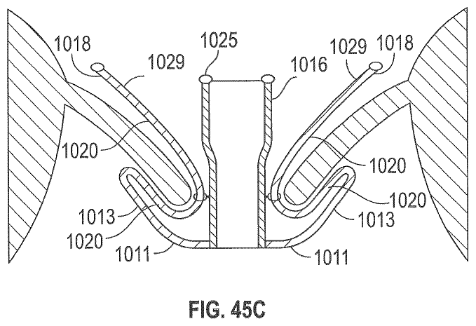

FIGS. 45A-C illustrate an example in which a radially inward portion 1011 of the second arm 1013 may be coupled to a valve body 1016. A radially outward portion of the second arm 1013 may be coupled to the loop of the tip. A proximal end 1018 (marked in FIG. 45C) of the first arm 1020 may comprise a free end of the first arm 1020.

Referring to FIG. 45A, the distal anchors may be in a stretched configuration and may extend axially along the valve body 1016. The valve body 1016 may include guides 1023 extending radially outward from the valve body 1016 that the distal anchors may pass through. The guides 1023, for example, may comprise loops or other structures for guiding the distal anchors. The distal anchors may slide along the valve body 1016 and through the guides 1023 to move to the deployed configuration. The distal anchors may be slid relative to the valve body 1016 by the valve body 1016 being retracted proximally, or the distal anchors being advanced distally.

FIG. 45B, for example, illustrates the distal anchors being deployed. The distal anchors, for example, may be held in position and the valve body 1016 may be retracted proximally. The distal anchors may expand radially outward. The anchors are configured to slide relative to the valve body to move from the stretched configuration to the deployed configuration. Similar to the example shown in FIGS. 40A-D, the distal anchors may also be retracted by controlling a relative sliding movement of the distal anchors. The arms may be controllable such that the one or more anchors are configured to move from the deployed configuration to the stretched configuration. For example, the valve body 1016 may be advanced relative to the distal anchors to cause the distal anchors to retract radially inward and may move the distal anchors to the stretched configuration. The position of the distal anchors may be readjusted until being provided in the desired position. The distal anchors may be repositioned to allow for recapture of leaflets, similar to the example shown in FIGS. 40A-D.

With the distal anchors in a desired position, the first arms 1020 may be released from the delivery system. FIG. 45C, for example, illustrates a configuration of the implant with the first arms 1020 released. A proximal end 1025 of the valve body 1016 may be released from the delivery system.

In examples, a radially inward portion of the first arm 1020 may be integral with a flange 1029 configured to extend radially outward from the valve body 1016. The flange 1029 may comprise a sealing body that may provide a seal with the native heart valve to reduce fluid flow. The flange 1029 may aid in reducing the possibility of the prosthetic valve moving in a distal or ventricular direction. The flange 1029 may comprise an atrial flange in examples that may impede distal or ventricular movement. The flange 1029 may be configured to be positioned on a proximal side of a native valve, with the distal anchors positioned on a distal side of the native valve.

In examples, the arms 1013, 1020 may be independently controllable between the stretched configuration and the deployed configuration. For example, the proximal ends 1018 of the first arms 1020 may be independently controlled to control deployment of each individual anchor. The anchors, for example, may each be configured to move from the stretched configuration to the deployed configuration, and from the deployed configuration to the stretched configuration, independent of another one of the plurality of anchors.

In examples, the arms may be shape set to move from the stretched configuration to the deployed configuration. The arms may self-expand. For example, the arms may be made of a shape memory material and configured to move to the deployed configuration. As such, upon release from the delivery system, the arms may move to a deployed configuration as shown in FIG. 45C, and FIGS. 40D and 41.

In examples, a control mechanism may be utilized to control a movement or deflection of the arms 1013, 1020. In examples, one or more actuators may be provided at a proximal portion of a delivery apparatus. A handle of a delivery apparatus, for example, may utilize one or more actuators (e.g., control knobs, or other forms of actuators) for allowing a user to actuate movement of one or more shafts of the delivery system coupled to the arms 1013, 1020. The one or more actuators, for example, may be coupled to a proximal portion of one or more shafts of the delivery system. The handle may have a configuration as shown in FIG. 10 or may have another configuration as desired. Other forms of control may be utilized in examples as desired.

The features of the examples of FIGS. 40A-45C may be utilized solely or in combination with any other example disclosed herein.

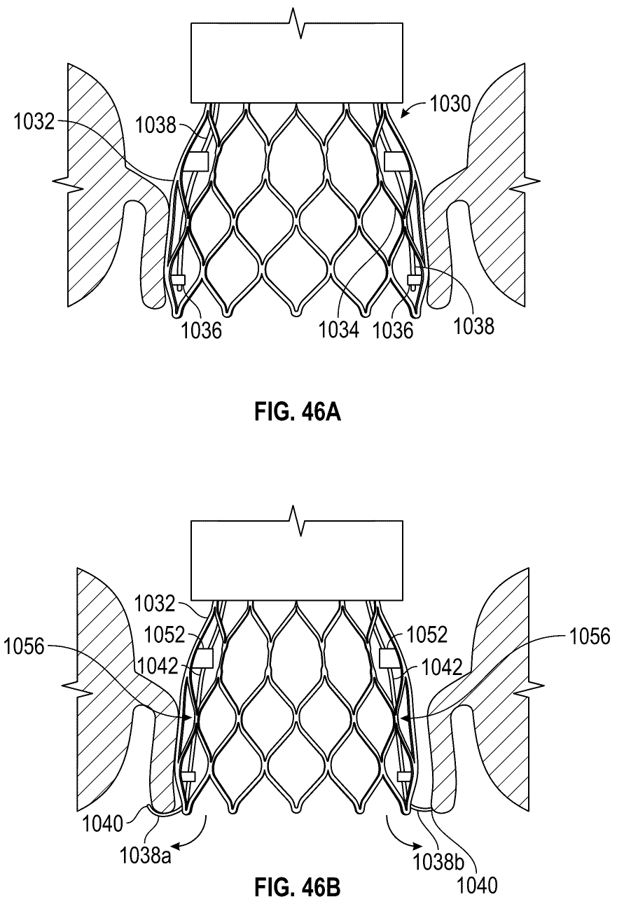

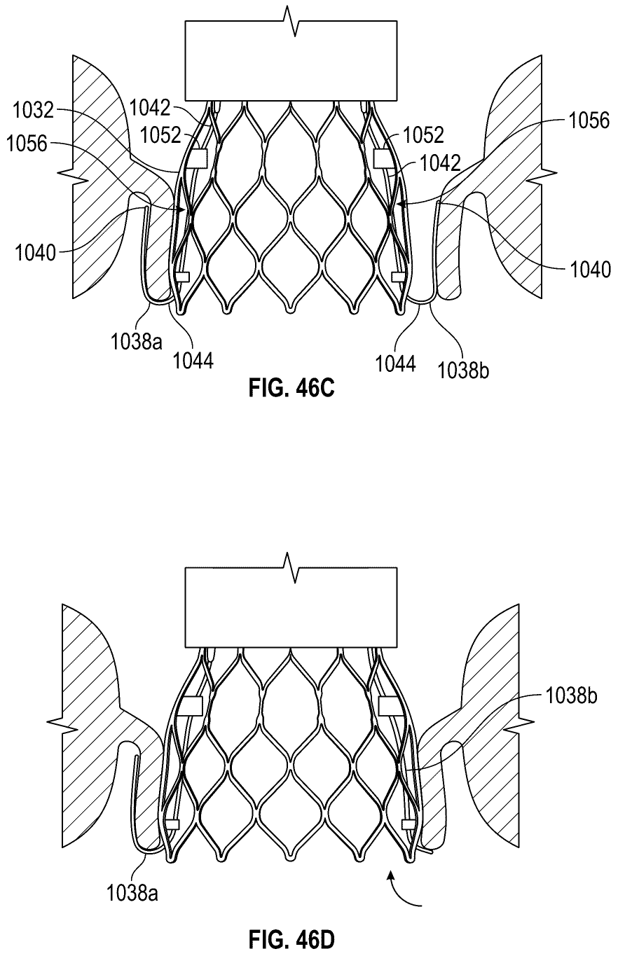

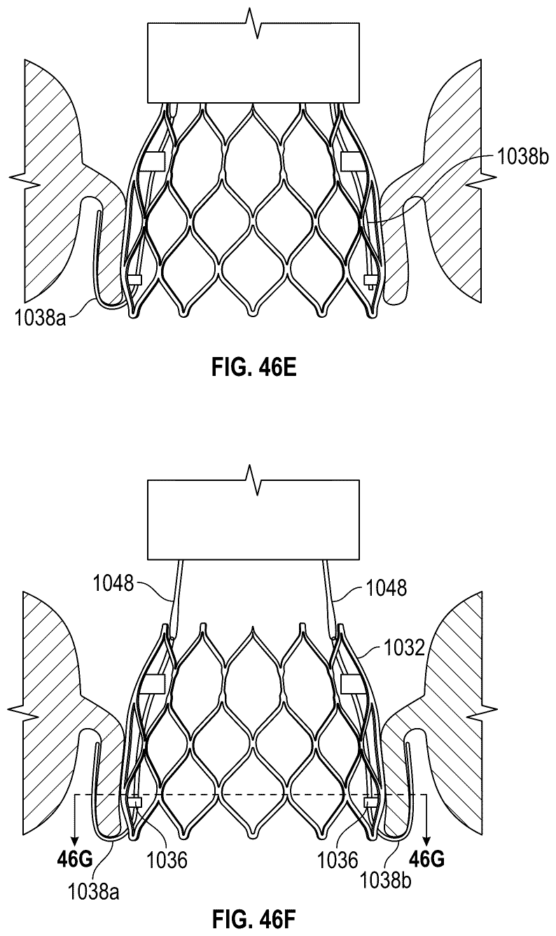

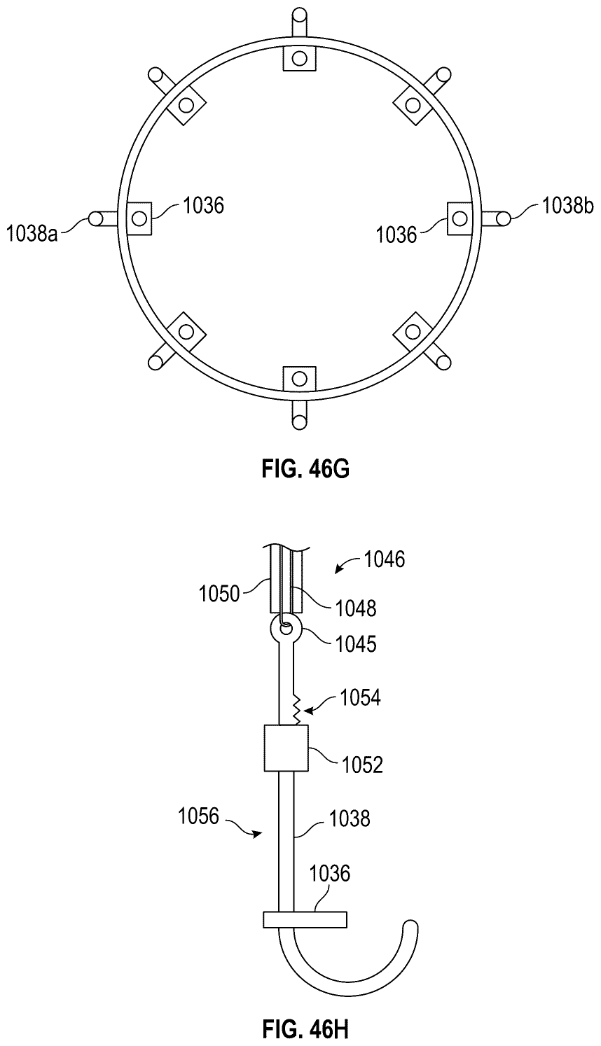

FIGS. 46A-F illustrate an example of a prosthetic valve in which anchors are each configured to anchor the prosthetic valve to a native valve and each configured to slide relative to the valve body. At least one of the anchors may be axially slidable relative to the valve body. FIG. 46A, for example, illustrates a prosthetic valve 1030 including a valve body 1032 having a valve frame 1034. In examples, the valve body 1032 may include one or more guides 1036 that may guide movement of the anchors. The prosthetic valve may be configured similarly as examples of prosthetic valves disclosed herein, and may include a plurality of prosthetic valve leaflets and may include a valve body supporting the plurality of prosthetic valve leaflets.

The anchors may comprise distal anchors 1038 that may comprise elongate arms configured to extend radially outward from the valve body 1032. Referring to FIG. 46C, each distal anchor 1038 may include a tip 1040 and a proximal portion 1042. Each of the anchors 1038 may extend radially outwardly to a tip 1040. The proximal portion 1042 may comprise a proximal arm of the respective anchor and may be slidably coupled to the valve body 1032. A bend portion 1044 may be positioned between the tip 1040 and the proximal portion 1042. The bend portion 1044 may form a hooked shape between the tip 1040 and the proximal portion 1042. Each distal anchor 1038 may be configured to hook over a distal tip or downstream end of a leaflet of a native valve. Each distal anchor 1038 may be configured to extend radially outward to a tip 1040 of the respective anchor.

Each distal anchor 1038 may be flexible and configured to move from an elongate configuration shown in FIG. 46A to a deployed configuration as shown in FIG. 46C. The distal anchors 1038 may each have a hooked shape in the deployed configuration. Each distal anchor 1038 may be configured to slide distally to allow the distal anchor 1038 to pass through the guide 1036 and extend radially outward from the valve body 1032. The proximal portion 1042 of the anchors may be configured to slide along the valve body 1032.

Similar to the examples of implants shown in FIGS. 3A-3E, the valve body 1032 may include a valve frame surrounding a central axis. The valve frame is shown in FIGS. 46A-46F, yet additional features such as prosthetic valve leaflets and a sealing body as discussed in regard to FIGS. 3A-3E may be utilized as desired. The anchors may slide relative to the valve body 1032 to vary an axial position of the tip 1040 of the anchor relative to the valve body 1032. The one or more anchors may be configured to be axially slidable relative to the valve frame. Each anchor may be slidable relative to the valve body for positioning the tip around a leaflet of the native heart valve.

Each distal anchor 1038 may be shape set to form a hooked configuration upon being passed radially outward from the valve body 1032. For example, the distal anchors 1038 may be made of a shape memory material that causes the distal anchor 1038 to form a hooked shape upon being passed radially outward from the valve body 1032. Each of the anchors may be formed with a hook shape. The distal anchors 1038 may move from having an elongate shape to having a hooked shape as shown in FIG. 46C. Each of the anchors 1038 may be shaped to extend around a downstream end of a leaflet of the native heart valve.

Referring to FIG. 46H, each distal anchor 1038 may include a proximal end 1045 that may be configured to couple to a control device 1046 for controlling movement of the anchor 1038. The control device 1046, for example, may include a tether 1048 for controlling retraction of the anchor 1038 and a pusher shaft 1050 for controlling advancement of the anchor 1038. The tether 1048 may be positioned within the pusher shaft 1050 in examples, or may be positioned external to the pusher shaft 1050 as desired. Each distal anchor 1038 may be controlled by a separate control device 1046 in examples.

A lock 1052 may be configured to lock a position of the distal anchor 1038 relative to the valve body 1032. The lock 1052 may secure a position of the distal anchors 1038 relative to the valve body 1032. The lock 1052, for example, may be configured to engage a locking portion 1054 of each distal anchor 1038. The lock 1052 may comprise one or more teeth, or a contoured body, that may engage the locking portion 1054. The locking portion 1054 may have a configuration that engages the lock 1052 and may have a corresponding shape in examples. In examples, the lock 1052 may comprise a ratcheting lock or ratcheting mechanism that allows for movement in one direction and impedes movement in an opposite direction.

Referring to FIG. 46A, the implant may be advanced to the implantation site and the distal anchors 1038 may be held in a retracted position. Upon the implant reaching the implantation site, the distal anchors 1038 may be slid distally to anchor to the implantation site. FIG. 46B, for example, illustrates the distal anchors 1038 having been advanced slightly. FIG. 46C, illustrates the distal anchors 1038 having been slid distally and expanded radially outward from the valve body 1032. The distal anchors 1038 may be slid distally to radially expand and may be slid proximally to be retracted. The distal anchors 1038 may slide such that a portion 1056 of the distal anchors 1038 distal of the locking portion 1054 may slide through the lock 1052. As such, the lock 1052 may avoid engaging the locking portion 1054 during the sliding movement of the distal anchors 1038.

The distal anchors 1038 may be slid distally and proximally relative to the valve body to allow the distal anchors 1038 to hook over a distal tip of the heart valve leaflet. The distance of the anchors from the valve body may be configured to vary. For example, as shown in FIG. 46C, an anchor 1038a may be advanced radially outward to hook over a heart valve leaflet. In addition, an anchor 1038b may be advanced radially outward to hook over a heart valve leaflet and may miss capture of the leaflet.

Referring to FIG. 46D, the distal anchor 1038b may be slid proximally to retract the distal anchor 1038b. The distal anchor 1038b accordingly may be retracted to allow the heart valve leaflet to move to a position for capture by the distal anchor 1038b. FIG. 46E illustrates the distal anchor 1038b retracted. The distal anchor 1038b may be advanced distally again to allow for the possibility of capture of the heart valve leaflet by the distal anchor 1038b. The distal anchor 1038b may be slid distally or proximally, or in a combination of distal and proximal movements, until the heart valve leaflet is captured.

FIG. 46F, for example, illustrates the distal anchor 1038b having been advanced to recapture the heart valve leaflet.

With the distal anchors in a desired position, the distal anchors may be locked in position relative to the valve body 1032. For example, each of the distal anchors 1038a, b may be advanced distally such that the locking portion 1054 engages the lock 1052. With the distal anchors locked in position, the respective tethers 1048 may be cut and withdrawn from the distal anchors. The tethers 1048 may be looped such that cutting an end of the tether may allow for withdrawal through the delivery system.

The anchors may each be configured to slide relative to the valve body independent of another one of the plurality of anchors. Each anchor may be independently controllable. Each anchor may be independently slidable for allowing the anchors to be positioned at different axial locations relative to the valve body 1032. In examples, the anchors 1038 may be provided in groups of two or more, and each group may be axially slidable independently of the other groups.

FIG. 46G illustrates a cross sectional view of the distal anchors 1038a, b along line 46G-46G in FIG. 46F. The heart valve leaflets are excluded from view in FIG. 46G. The distal anchors are shown to pass through the guides 1036 of the valve body.

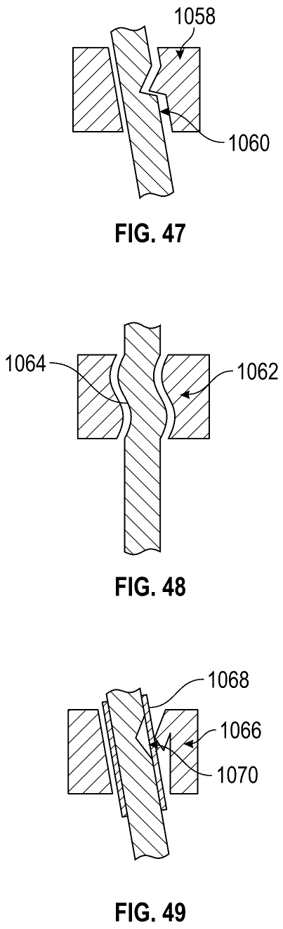

FIG. 47 illustrates an example of a lock 1058 that may be utilized in examples herein. The lock 1058 may comprise a ratcheting lock that engages a locking portion 1060 of a distal anchor. The distal anchor, for example, may be able to slide distally and the lock 1058 may engage the locking portion 1060 to impede proximal movement of the distal anchor.

FIG. 48 illustrates an example of a lock 1062 that may be utilized in examples herein. The lock 1062 may have a contour that may match a locking portion 1064 of a distal anchor having a corresponding contour. The locking portion 1064 may fit into and engage the lock 1062.

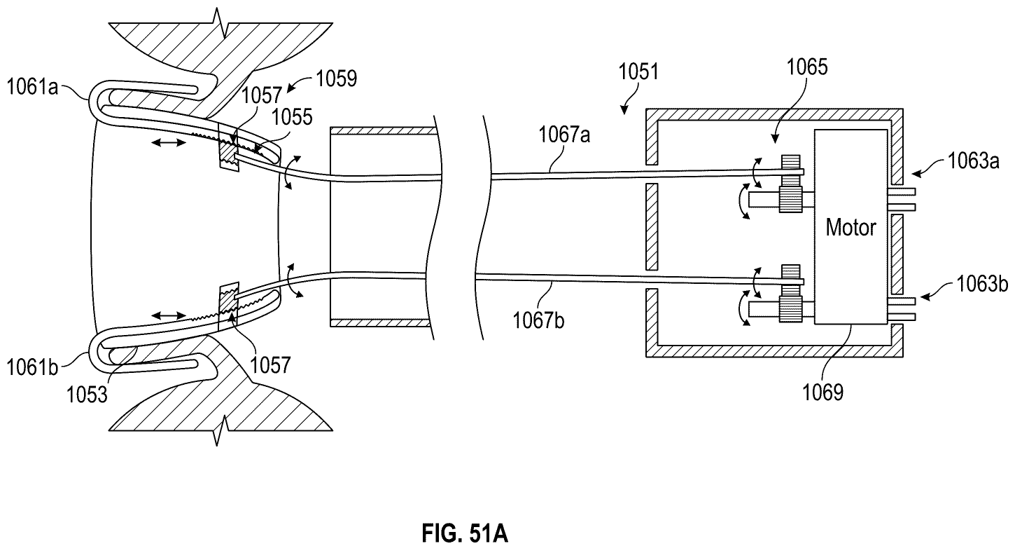

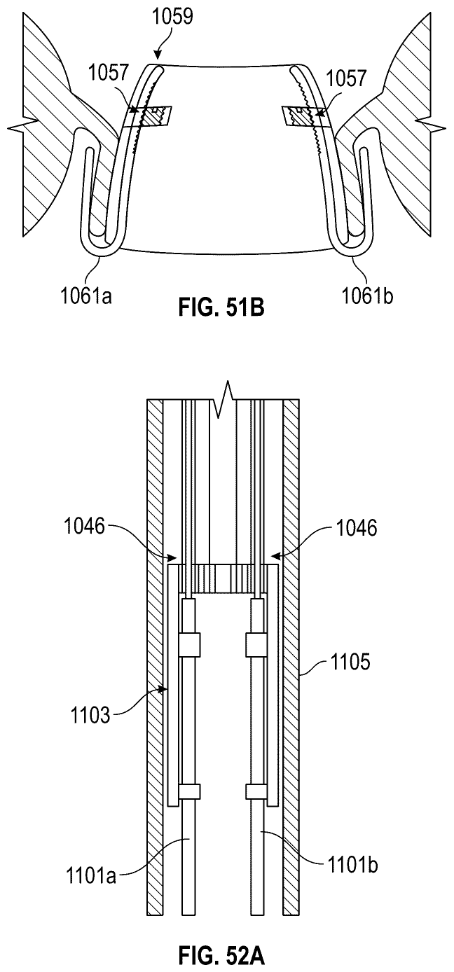

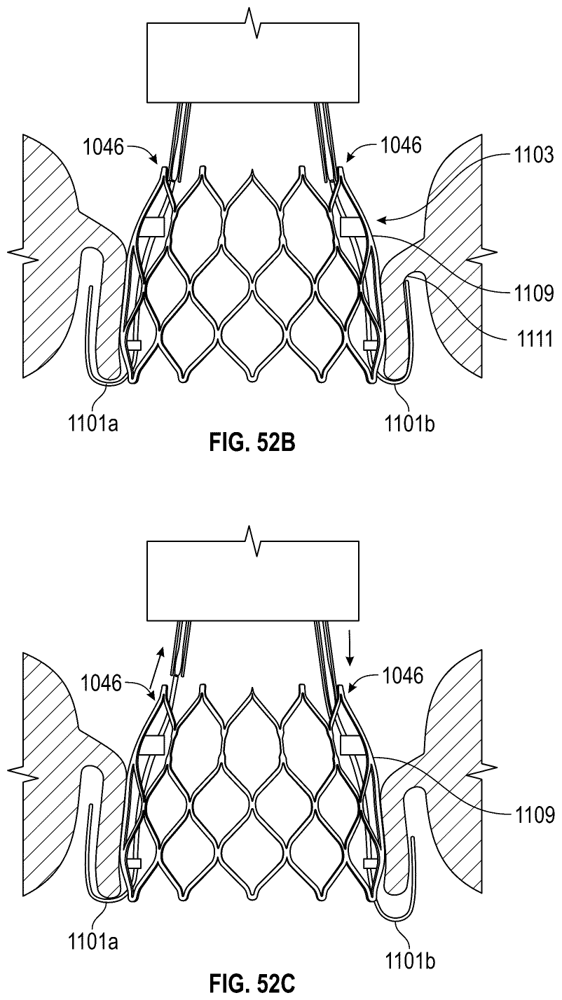

FIG. 49 illustrates an example of a lock 1066 that may be selectively controlled. A sheath 1068, for example, may extend over the distal anchor and may impede the engagement of the lock 1066 with the locking portion 1070. Upon withdrawal of the sheath 1068 at a desired time, the lock 1066 may engage the locking portion 1070 and lock the distal anchor in position. Other forms of locks such as actuating fins or other forms of locks may be utilized in examples. Locks may be selectively controlled in examples or may comprise passive locks in examples. In examples, a control mechanism as disclosed herein may be utilized to operate a lock. The lock may comprise a selectively controlled lock. For example, a control mechanism as shown in FIG. 51 or 52 may be configured to operate a lock, such as a selectively controlled lock as desired.

In examples, a control mechanism may be utilized for actuating at least one of the anchors 1038 to axially slide relative to the valve body 1032. The control mechanism may have a variety of forms. For example, a control mechanism may include features of the control mechanism shown in FIGS. 54-63 as desired. Such a control mechanism may be utilized to slide the tether 1048 and pusher shaft 1050 proximally or distally, for example, to actuate the anchors 1038 to axially slide relative to the valve body 1032. Other forms of control mechanisms may be utilized as desired.

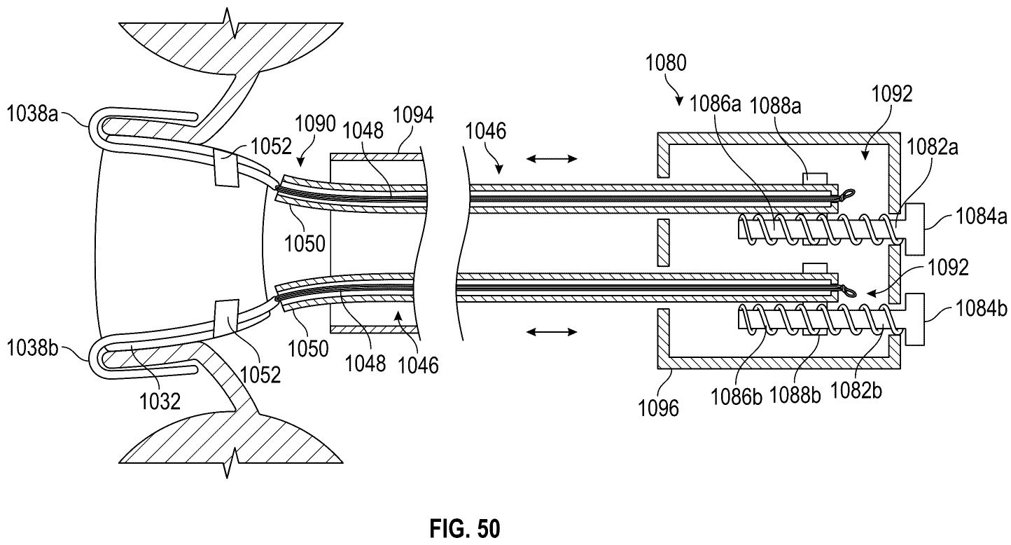

For example, FIG. 50 illustrates a control mechanism 1080 for actuating at least one of the anchors 1038 to axially slide relative to the valve body 1032. The control mechanism 1080 may be operable to actuate at least one of the anchors 1038a, b to slide proximally or distally relative to the valve body 1032. As shown in FIG. 50, the control mechanism 1080 may include one or more of the control devices 1046, which may be in the form of a tether 1048 and pusher shaft 1050 for moving the anchors 1038a, b proximally or distally. The tether 1048 and pusher shaft 1050 may actuate the anchors 1038a, b to axially slide relative to the valve body 1032. Other forms of control devices may be utilized as desired.

The control mechanism 1080 may include one or more actuators 1082a, b that may be operable to axially slide at least one of the anchors 1038a, b. The actuators 1082a, b may be operable by a user to axially slide at least one of the anchors 1038a, b. For example, the actuators 1082a, b may have the form of a control knob that may be manipulated by a user. The control knob may have a portion 1084a, b that is positioned external of a portion of a delivery apparatus, such as a handle of a delivery apparatus. A user may operate the actuators 1082a, b to axially slide at least one of the anchors 1038a, b. Other configurations of actuators 1082a, b may be utilized in examples.

The actuators 1082a, b may be configured to move the control devices 1046 proximally or distally. For example, the actuators 1082a, b may include a threaded portion 1086a, b that may be configured to engage a threading on a respective drive body 1088a, b coupled to a control device 1046. As such, rotation of an actuator 1082a, b in a first direction may slide the control device 1046 proximally and rotation of an actuator 1082a, b in a second opposite direction may slide the control device 1046 distally. The sliding movement of the control devices 1046 accordingly may slide the anchors 1038a, b. In examples, the control mechanism 1080 may be configured to control a lock for the anchors 1038. In an example in which a lock is selectively controlled, as shown in FIG. 49, for example, the control mechanism 1080 may be configured to retract the sheath 1068. Other forms of control may be provided as desired.

The control mechanism 1080 may be operable to actuate the anchor 1038a to axially slide relative to the valve body 1032, and may be operable to actuate the anchor 1038b to axially slide relative to the valve body 1032 independent from the anchor 1038a. As such, independent control of anchors 1038a, b or groups of anchors 1038 may be provided.

The control mechanism 1080 may include an engagement portion 1090 for engaging at least one of the anchors 1038a, b. For example, the engagement portion 1090 may comprise the portion of the tethers 1048 that engage the anchors 1038a, b. The engagement portion 1090 may be configured to release from the anchors 1038a, b upon deployment of the prosthetic valve to the native heart valve. For example, the tethers 1048 may be cut to release from the anchors 1038a, b. FIG. 50 illustrates a knot portion 1092 that may be accessible by a user to cut and release the tethers 1048 upon deployment.

In examples, the control mechanism 1080 may comprise a portion of a delivery apparatus for delivering the prosthetic valve to an implantation site. The control mechanism 1080, for example, may extend along the elongate shaft 1094 of the delivery apparatus. The actuators 1084a, b may be positioned at a proximal end portion of the elongate shaft 1094 or the handle 1096 of the delivery apparatus as desired. Portions of the control mechanism may have other positions in examples as desired. The delivery apparatus may include other features, such as features discussed in regard to the delivery system 10. Other configurations of delivery apparatuses may be utilized in examples.

Other forms of control mechanisms may be utilized in examples. FIG. 51A, for example, illustrates a control mechanism 1051 utilizing a gear drive for actuating the anchors 1061a, b to axially slide relative to the valve body 1053. The gear drive, for example, may include an engagement portion 1055 configured to engage with a displacement mechanism 1057 of the prosthetic valve 1059. The displacement mechanism 1057, for example, may comprise a worm gear or other form of displacement mechanism for actuating the sliding movement of the anchors 1061a, b. A rotation of the worm gear in a first direction may cause a distal slide of one of the anchors 1061a, b and a rotation of the worm gear in a second opposite direction may cause a proximal slide of the respective anchor 1061a, b. The engagement portion 1055 may comprise a distal end portion of the control mechanism 1051 that may engage the worm gear (e.g., mate with a protrusion or recess of the worm gear).

One or more actuators 1063a, b may be provided for actuating the control mechanism 1051. Each actuator 1063a, b for example, may be configured to actuate each anchor 1061a, b individually. A gear system 1065, for example, may rotate respective drive shafts 1067a, b to produce axial movement of the respective anchors 1061a, b.

In examples, the control mechanism 1051 may be motorized, and may include a motor 1069 for actuating movement of the anchors 1061a, b. In examples, the motor 1069 may receive an input from the actuators 1063a, b. The motor 1069 may be configured to independently actuate the anchors 1061a, b. The motor 1069 may operate the gear system 1065 as desired.

Components of the control mechanism 1051 may be positioned in similar locations as with the control mechanism 1080.

Upon deployment of the prosthetic valve 1059 to the native heart valve, the engagement portion 1055 may be configured to release from the anchors 1061a, b. For example, the distal end portions of the drive shafts 1067a, b may be retracted from the displacement mechanism 1057. FIG. 51B illustrates a configuration of the prosthetic valve 1059 deployed, with the drive shafts 1067a, b retracted.

Other forms of control mechanisms may be utilized in examples. For example, hydraulic, electrical, magnetic, or thermal control mechanisms may be utilized as desired. Other forms of mechanical devices (e.g., pulleys, wheel drives, pistons, scissor arms, among others) may be utilized as desired.

In examples, the anchors may be formed with a hook shape such that the hook shape is retained upon axial sliding of the anchors. For example, the anchors may be shape set or otherwise formed into a hook shape that is retained. The anchors may be deployed with the hook shape in examples.

For example, referring to FIG. 52A, the anchors 1101a, b may be elongated in an undeployed or compressed configuration. The undeployed or compressed configuration may comprise a configuration in which the prosthetic valve 1103 is retained within a capsule 1105 of a delivery apparatus for example.

Upon movement to a deployed or expanded configuration, the anchors 1101a, b may expand radially outward to the hook shape. For example, FIG. 52B illustrates the anchors 1101a, b having the hook shape. The anchors 1101a, b, upon proximal or distal movement, may retain the hook shape. The anchors 1101a, b may be slid axially relative to the valve body 1109 utilizing any of the methods disclosed herein.

For example, the anchor 1101b may be slid distally to account for the contact point 1111 with the valve annulus. The anchor 1101a may be slid proximally to adjust the distance of the anchor 1101a from the native valve leaflet. FIG. 52C illustrates a resulting adjustment in the positions of the anchors 1101a, b. As such, a lower position of the anchor 1101b relative to the valve body may be provided upon distal movement of the anchor 1101b, and a raised position of the anchor 1101a may be provided relative to the valve body upon proximal movement of the anchor 1101a.

The anchors 1101a, b may be moved for a variety of purposes, including to address a geometry of the native heart valve or to address failed capture of a leaflet. The anchors 1101a, b may be adjusted to provide a fit of the prosthetic valve 1103 to the native valve, which may be to provide improved scaling with the native valve or to address contact of an anchor with a portion of the native valve. For example, the contact point 1111 of the anchor 1101b to the native valve may result in a conduction disturbance of the native valve, which may be undesirable. The anchor 1101b position may be adjusted to reduce the possibility of such conduction disturbance.

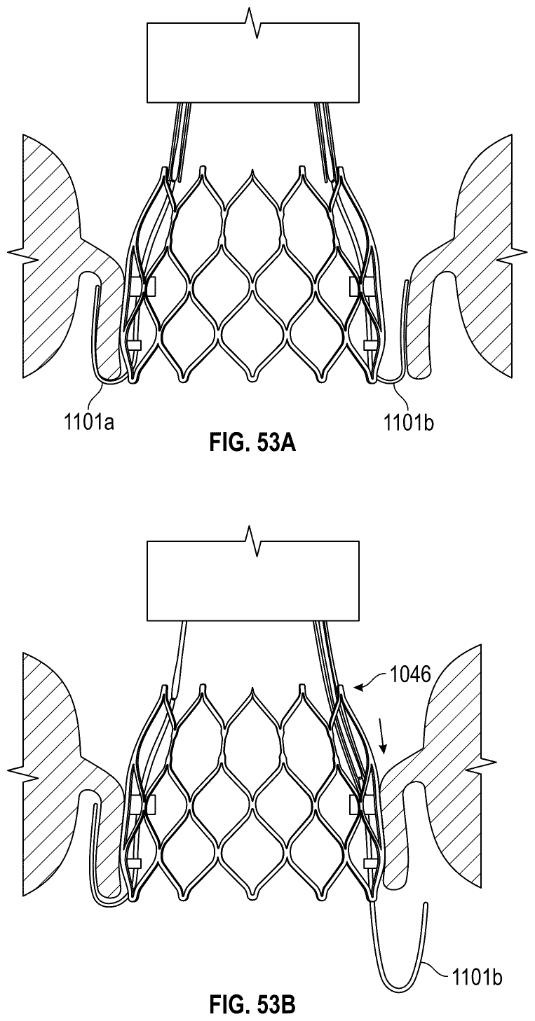

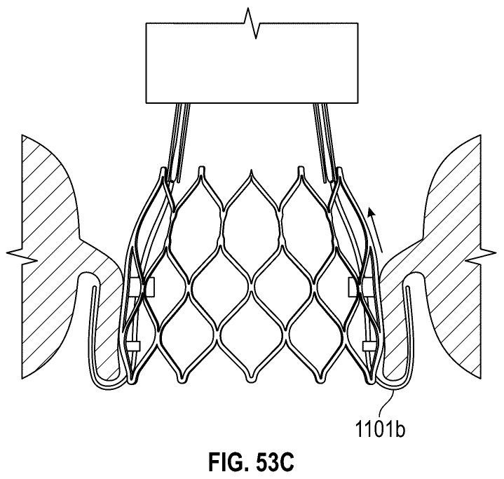

FIG. 53A illustrates an example of a failed capture of a leaflet by the anchor 1101b. Referring to FIG. 53B, the anchor 1101b may be slid distally to allow the leaflet to move radially inward and be recaptured by the anchor 1101b. Referring to FIG. 53C, the anchor 1101b may be retracted proximally for capture of the native valve leaflet. The anchors 1101a, b may retain the hook shape upon proximal or distal movement of the anchors 1101a, b.

The features of the examples of FIGS. 46A-53C may be utilized solely or in combination with any other example disclosed herein.

FIG. 54 illustrates a schematic view of a delivery system 1100 for an implant. The delivery system 1100 may include a control mechanism 1102. The control mechanism 1102 may be configured to control a deflection of at least one distal anchor 1104a of an implant 1106 independent of a deflection of at least one other distal anchor 1104b of the implant 1106.

The implant 1106 may have a variety of forms. For example, the implant 1106 may be configured similarly as the implant 915 shown in FIG. 33B. The implant 1106 may have the form of any other implant disclosed herein. The implant 1106 may have other forms in examples as desired.

The control mechanism 1102 may include a coupler assembly 1107. The coupler assembly 1107 may be configured to couple the anchors 1104a, b of the implant 1106 to a drive assembly 1118 in examples.

The coupler assembly 1107 may comprise a tether assembly in examples and may include one or more tethers 1108a, b that may be utilized to control deflection of the anchors 1104a, b independent from each other. For example, each tether 1108a, b may include a respective distal portion 1110a, b and a proximal portion 1112a, b. A first tether 1108a may be configured to control deflection of the at least one distal anchor 1104a. A second tether 1108b may be configured to control deflection of the at least one other distal anchor 1104b.

The distal portions 1110a, b of the tethers 1108a, b may be configured to couple to a respective portion of the implant 1106. For example, each distal portion 1110a, b may be configured to couple to one or more of the anchors for control of the respective anchor. The distal portion 1110a, for example, may couple to the anchor 1104a. The distal portion 1110b may couple to the anchor 1104b. The distal portion 1110a may be configured to move the anchor 1104a independent from the other anchor 1104b. Similarly, the distal portion 1110b may be configured to move the anchor 1104b independent from the anchor 1104a.

In examples, the distal portion 1110a may be configured to couple to a first plurality of anchors and control the first plurality of anchors independent of a second plurality of anchors. Similarly, the distal portion 1110b may be configured to couple to the second plurality of anchors and control the second plurality of anchors independent of a first plurality of anchors.

The tethers 1108a, b may control the respective anchors 1104a, b by applying a compressive force to the anchors 1104a, b. The tethers 1108a, b may be configured to couple to the respective anchors 1104a, b and apply the compressive force radially inward. Such compressive methods may include methods disclosed herein. For example, the tethers 1108a, b may be routed relative to the implant 1106 such that a compressive force to the respective anchors 1104a, b is caused by proximal tension in the tethers 1108a, b. In examples, an elongate shaft 1114 may be configured for the tethers 1108a, b to pass through. The elongate shaft 1114 may be positioned such that the tethers 1108a, b being pulled through the elongate shaft 1114 may apply the compressive force to the anchors 1104a, b. The proximal portions 1112a, b may be configured to be retracted proximally through the elongate shaft 1114 to cause the tethers 1108a, b to apply the compressive force to the respective anchors 1104 radially inward.

In examples, a portion of the elongate shaft (e.g., an inner shaft 1113) may pass through the implant 1106 or the flow channel of the implant to be positioned co-planar with the anchors 1104a, b. As such a proximal force applied to the tethers 1108a, b may comprise a compressive force applied to the anchors 1104a, b. Other forms of control or routing may be utilized in examples.

The proximal portions 1112a, b of the respective tethers 1108a, b may be configured to couple to a drive assembly 1118 of the control mechanism 1102. The proximal portions 1112a, b may be configured to pass through the elongate shaft 1114, for example, and the distal portions 1110a, b may be configured to couple to the distal anchors 1104a, b of the implant.

The tethers 1108a, b may include materials having a variety of forms. For example, a distal portion 1110a, b of the tethers 1108a, b may comprise a suture. A proximal portion 1112a, b may comprise a wire or may have another form as desired. A respective coupler 1120a, b may be configured to couple the distal portion 1110a, b of the tethers 1108a, b to the proximal portions 1112a, b. The tethers 1108a, b may have other forms in examples.

In examples, the coupler assembly 1107 may have other forms as desired.

The delivery system 1100 may include the elongate shaft 1114. The elongate shaft 1114 may be configured to advance the implant 1106 to the implantation site. The elongate shaft 1114 may be configured similarly as examples of shafts of delivery systems disclosed herein, or may have another configuration as desired. The elongate shaft 1114, for example, may be configured to be deflectable to a desired position to orient the implant 1106 as desired at the implantation site (e.g., the native valve). The elongate shaft 1114 may include an implant retention area for retaining the implant 1106. The implant retention area, for example, may comprise a capsule 1115 for surrounding the implant 1106 or may have another form in examples. In examples, a capsule 1115 may be excluded and other forms of implant retention areas may be utilized.

The elongate shaft 1114 may include a plurality of shafts or sheaths. For example, the elongate shaft 1114 may include a first inner shaft 1113 or tether shaft. The first inner shaft 1113 may include an interior lumen 1122 that the tethers 1108a, b may extend along. A second inner shaft 1124, or coupler shaft, may be positioned within the interior lumen 1122. One or more couplers 1116 may be coupled to the inner shaft 1124 in examples.

The elongate shaft 1114 may include a distal end portion 1126 that the implant 1106 may be deployed at. The elongate shaft 1114 may extend to a proximal end portion 1128. In examples, the proximal end portion 1128 may be configured to be positioned outside of a patient's body during an implantation procedure. The elongate shaft 1114 may extend within the patient's vasculature with the proximal end portion 1128 accessible by a user. In examples, the proximal end portion 1128 may be located within a patient's body during an implantation procedure.

The drive assembly 1118 of the control mechanism 1102 may be utilized to drive the coupler assembly 1107. The drive assembly 1118, for example, may be configured to tension or release one or more tethers 1108a, b. The drive assembly 1118 may be configured to be operated to deflect the at least one distal anchor 1104a of an implant 1106 independent of a deflection of at least one other distal anchor 1104b of the implant 1106. The drive assembly 1118 may have a variety of forms.

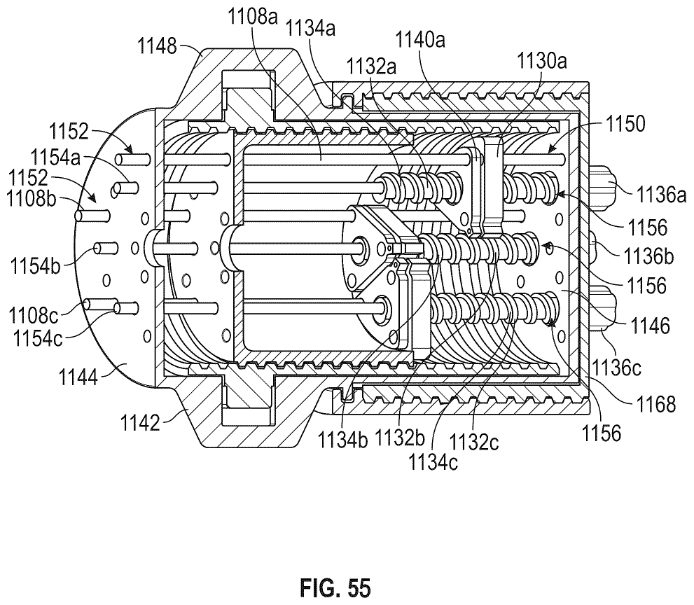

For example, referring to FIG. 54, the drive assembly 1118 may include one or more drive bodies 1130a, b, c. The drive bodies 1130a, b, c may each be configured to couple to a proximal end portion of one of the tethers (a tether 1108c that drive body 1130c may couple to is excluded from view for clarity in FIG. 54 and is marked in FIG. 55). For example, the drive body 1130a may couple to the proximal portion 1112a of the tether 1108a. The drive body 1130b may couple to the proximal end portion 1112b of the tether 1108b. The drive body 1130a may be configured to control deflection of the anchor 1104a. The drive body 1130b may be configured to control deflection of the anchor 1104b. Additional drive bodies may be provided in examples. For example, a drive body may be provided for each corresponding tether if desired (e.g., six drive bodies for six tethers; nine drive bodies for nine tethers, etc.).

The drive bodies 1130a, b, c may each be configured to move distally or proximally. A distal movement may move a respective tether distally, to release the respective tether distally. A proximal movement may move a respective tether proximally, to retract the respective tether proximally. A distal movement of a tether 1108a, for example, may allow the anchor 1104a to expand radially outward or allow for a greater curvature of a hooked shape of the anchor 1104a. The anchor 1104a may move from an elongated configuration to a hooked configuration. The anchors 1104a, b may be biased towards the hooked configuration or the configuration with a greater curvature in examples. A proximal movement of the tether 1108a, for example, may overcome the bias and provide a compressive force to the anchor 1104a or may move the anchor 1104a radially inward. The anchor 1104a may have a lesser curvature of a hooked shape of the anchor 1104a, or may have a more elongate shape. An elongated configuration may be similar to configurations disclosed herein (e.g., towards an elongated configuration shown in FIG. 33A for example). The anchor 1104a may move from a hooked configuration to an elongated configuration.

In examples, the control mechanism 1102 may include at least one actuator 1132a, b, c for controlling the deflection of at least one distal anchor of the implant. The one or more actuators 1132a, b, c, may be provided that may be configured to move a respective one of the drive bodies 1130a, b, c. The actuators 1132a, b, c may have a variety of forms in examples. Referring to FIG. 54, in examples, the actuators 1132a, b, c may comprise a respective drive shaft 1134a, b, c and may include a respective control surface 1136a, b, c. The drive shaft 1134a, b, c may comprise a threaded shaft in examples, and may be configured such that rotation of the drive shaft 1134a, b, c may move the respective drive body 1130a, b, c. The control surface 1136a, b, c may comprise a surface for a user to manipulate to control the drive shaft 1134a, b, c and the respective drive body 1130a, b, c. For example, each control surface 1136a, b, c may comprise a knob that allows for rotation of the respective drive shaft 1134a, b, c or may have another configuration as desired.

In examples, the drive bodies 1130a, b, c may be non-threaded and may each include a respective opening that may allow the drive body 1130a, b, c to move along the drive shaft 1134a, b, c. The drive bodies 1130a, b, c accordingly may lack a threaded engagement with the respective drive shafts 1134a, b, c in examples and may slide along the drive shafts 1134a, b, c.

In examples, adaptors 1140a, b, c may be utilized that transmit force from the drive shaft 1134a, b, c to a respective one of the drive bodies 1130a, b, c. For example, each adaptor 1140a, b, c may include threading that may engage the threading of the drive shaft 1134a, b, c. Each adaptor 1140a, b, c may move longitudinally upon the rotation of the respective drive shaft 1134a, b, c. A proximal movement of an adaptor 1140a, b, c may press the respective drive body 1130a, b, c proximally. A distal movement of an adaptor 1140a, b, c may allow the respective drive body 1130a, b, c to move distally due to a tension force applied by the respective tether.

In examples, the control mechanism 1102 may include a housing 1142. The housing 1142 may be configured to retain components of the drive assembly 1118 or other components as desired. Referring to FIG. 55, the housing 1142 may include a distal surface 1144 and a proximal surface 1146. The housing 1142 may include a side surface 1148 or outer surface. The side surface 1148 or outer surface may be configured for a user to grip in examples. The housing 1142 may include an interior cavity 1150 that may be configured to retain components of the drive assembly 1118.

The distal surface 1144 of the housing 1142 may include one or more openings 1152 that may allow components of the control mechanism 1102 to pass through. For example, the tethers 1108a-c may pass through the distal surface 1144 of the housing 1142. Pivots 1154a, b, c, that the respective drive shafts 1134a, b, c may rotate about may pass through the distal surface 1144 of the housing 1142 as desired.

The proximal surface 1146 of the housing 1142 may include one or more openings 1156. The openings 1156 may be configured for a respective one of the actuators 1132a, b, c to pass through. The openings 1156 may be non-threaded in examples. The respective drive shafts 1134a, b, c may pass through the non-threaded openings such that the control surfaces 1136a, b, c are accessible exterior of the housing 1142.

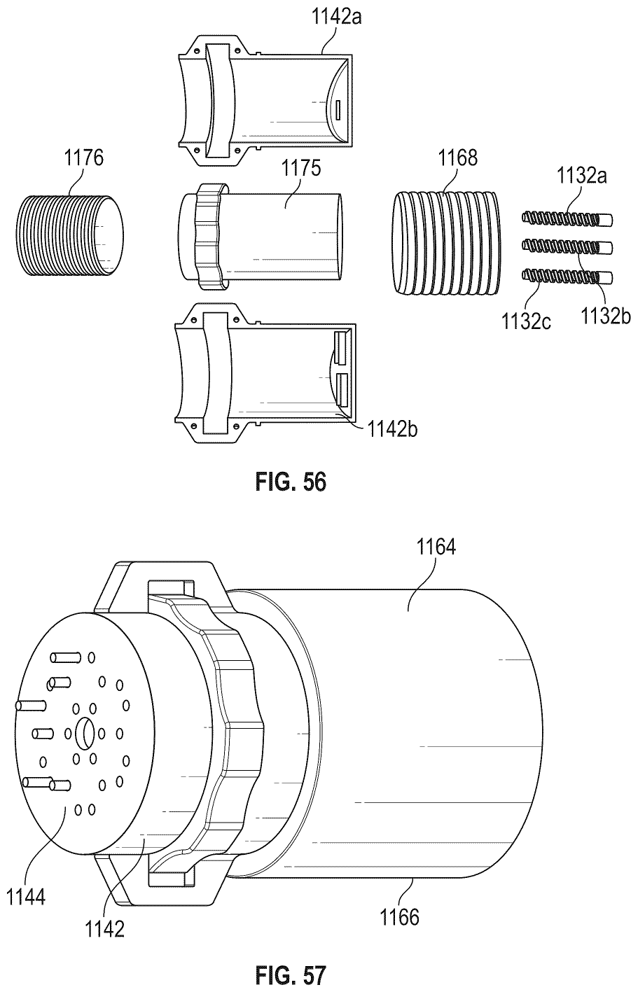

The housing 1142 may be formed of one or more shells that may retain components within the interior cavity 1150. FIG. 56, for example, illustrates an assembly view of components of the control mechanism 1102. Shells 1142a, b of the housing 1142 are shown to comprise halves of the housing 1142.

FIG. 57 illustrates an exterior perspective view of the housing 1142.

The housing 1142 may be positioned to be accessible by a user in examples. For example, the housing 1142 may be configured to be positioned exterior of a patient's body during an implantation procedure. The housing 1142 may be integrated with or coupled to a housing that may be utilized to control other features of the delivery system 1100 such as deflection of the elongate shaft 1114 or release of the implant 1106, among other features. The housing 1142 may be positioned in-line with other components of a housing or handle as shown in FIG. 10, for example. In examples, the housing 1142 may be utilized separate from other components of a housing or handle. Various other positions of the housing 1142 may be utilized.

In operation, the control mechanism 1102 may control deflection of at least one distal anchor 1104a of an implant 1106 independent of a deflection of at least one other distal anchor 1104b of the implant 1106 to account for a missed capture of a leaflet of a native valve or to account for a shape of the implantation site (e.g., a shape of the native valve).

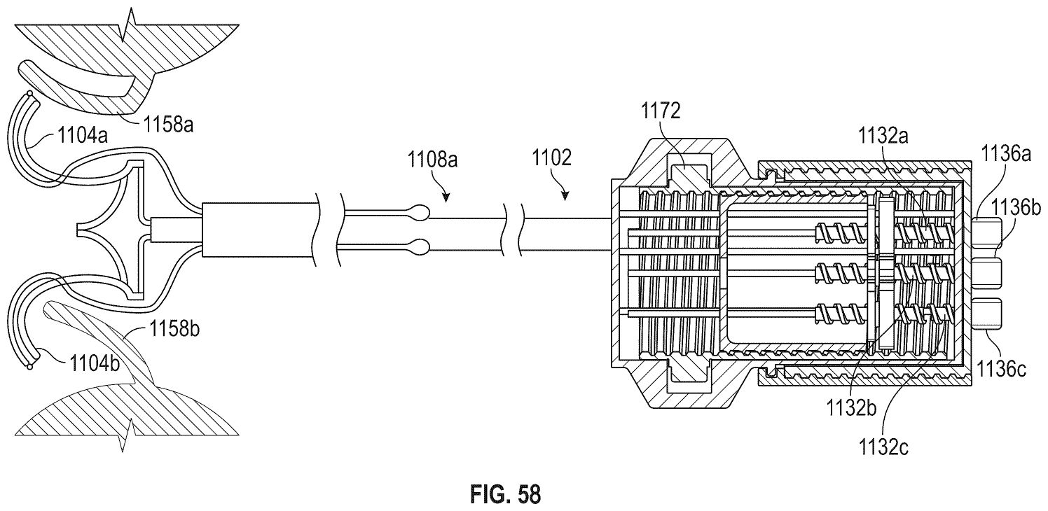

For example, referring to FIG. 58, a deployment of one or more of the anchors 1104a, b may result in a missed capture of the leaflet 1158a and a capture of a leaflet 1158b. At this point in a procedure, a user may need to decide whether to retract or recapture all anchors in an example in which all anchors are controlled together. However, utilizing the control mechanism 1102, independent control of anchors 1104a, b may be provided. As such, a user may deflect the anchor 1104a to attempt to recapture the leaflet 1158a without releasing capture of the leaflet 1158b. Various other benefits to the control mechanism 1102 may be provided in a deployment procedure. For example, a user may adjust a shape of an anchor 1104a to account for a longer shape of a leaflet 1158a while retaining a shorter geometry for the anchor 1104b based on a shorter leaflet 1158b.

FIG. 55 illustrates an exemplary use of the control mechanism 1102 to independently control deflection of at least one anchor 1104a. Referring to FIG. 55, for example, the actuator 1132a has been operated to retract the drive body 1130a. The adaptor 1140a presses proximally against the drive body 1130a to retract the drive body 1130a. The tether 1108a accordingly has been retracted independent of the tethers 1108b, c.

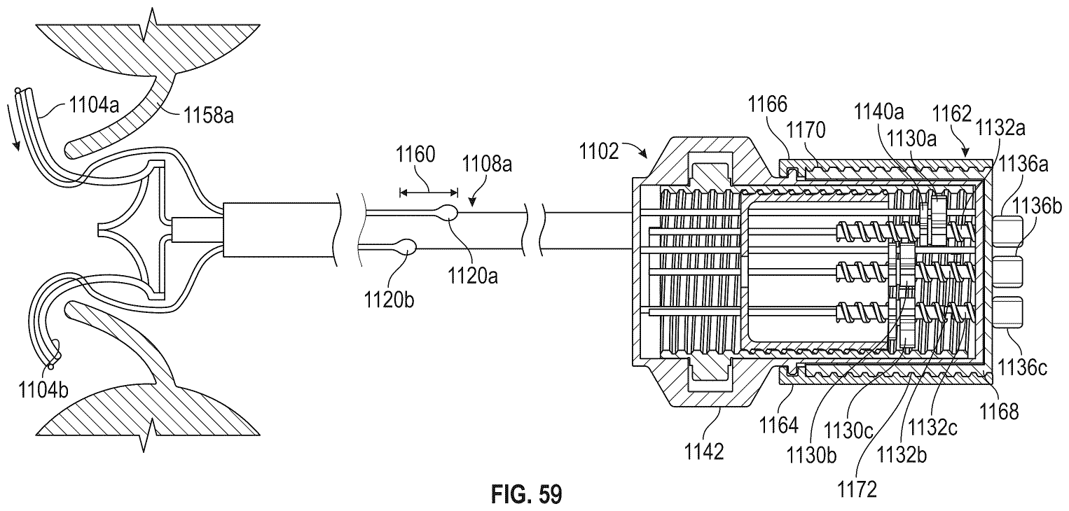

Referring to FIG. 59, the retraction of the tether 1108a by a distance or length 1160 is shown. The anchor 1104a accordingly deflects distally or inward to allow for recapture of the leaflet 1158a. The anchor 1104b may remain in the same position upon movement of the anchor 1104a. An offset in the position of the anchors 1104a, b and tethers 1108a, b may be provided.

A user accordingly may utilize the actuator 1132a to attempt to recapture the leaflet 1158a. The actuator 1132a may accordingly cause the adaptor 1140a to move distally. A distal tension provided by the tether 1108a may cause the drive body 1130a to slide distally and accordingly allow the anchor 1104a to return to a position having a greater curvature as shown in FIG. 58 for example.

In examples, the control mechanism 1102 may include an offset controller 1162. The offset controller 1162 may be configured to control the deflection of at least one of the anchors 1104a simultaneously with the deflection of the at least one other anchor 1104b of the implant with a deflection offset between the at least one distal anchor 1104a and the at least one other distal anchor 1104b. The offset in the position of the anchors 1104a, b may be maintained.

The offset controller 1162, for example, may include an actuator 1164 for moving the drive bodies 1130a, b, c. The offset controller 1162 may be configured to move the drive bodies 1130a, b, c simultaneously. The actuator 1164 may slide the drive bodies 1130a, b, c proximally simultaneously while maintaining an offset in the position of the drive bodies 1130a, b, c. The actuator 1164 may allow the drive bodies 1130a, b, c to slide distally simultaneously while maintain an offset in the position of the drive bodies 1130a, b, c.

The actuator 1164 may include a control surface 1166 and a pressing body 1168. The control surface 1166 may be configured to be operated by a user to actuate the actuator 1164. The control surface 1166, for example, may comprise an outer grip surface upon the housing 1142. The control surface 1166 may be rotated. The control surface 1166 may include threading 1170 that may be configured to engage with threading 1172 on the pressing body 1168. The control surface 1166 may be coupled to the housing 1142 such that the axial position of the control surface 1166 does not vary upon rotation of the control surface 1166.

The pressing body 1168 may be positioned interior of the control surface 1166. The pressing body 1168 may be configured to slide axially along the housing 1142 upon rotation of the control surface 1166. The pressing body 1168 may include an outer surface having the threading 1172. As such, upon rotation of the control surface 1166, the pressing body 1168 may be moved axially proximal or distal.

The pressing body 1168 may be configured to contact the control surfaces 1136a, b, c of the actuators 1132a, b, c. The control surfaces 1136a, b, c of the actuators 1132a, b, c may each have a greater diameter than openings of the pressing body 1168 and as such may be pressed by the surface of the pressing body 1168 upon movement of the pressing body 1168. Notably, the openings of the pressing body 1168 and the openings 1156 of the proximal surface 1146 of the housing 1142 may be unthreaded such that the drive shafts 1134a, b, c may freely slide through the openings upon axial movement of the control surfaces 1136a, b, c.

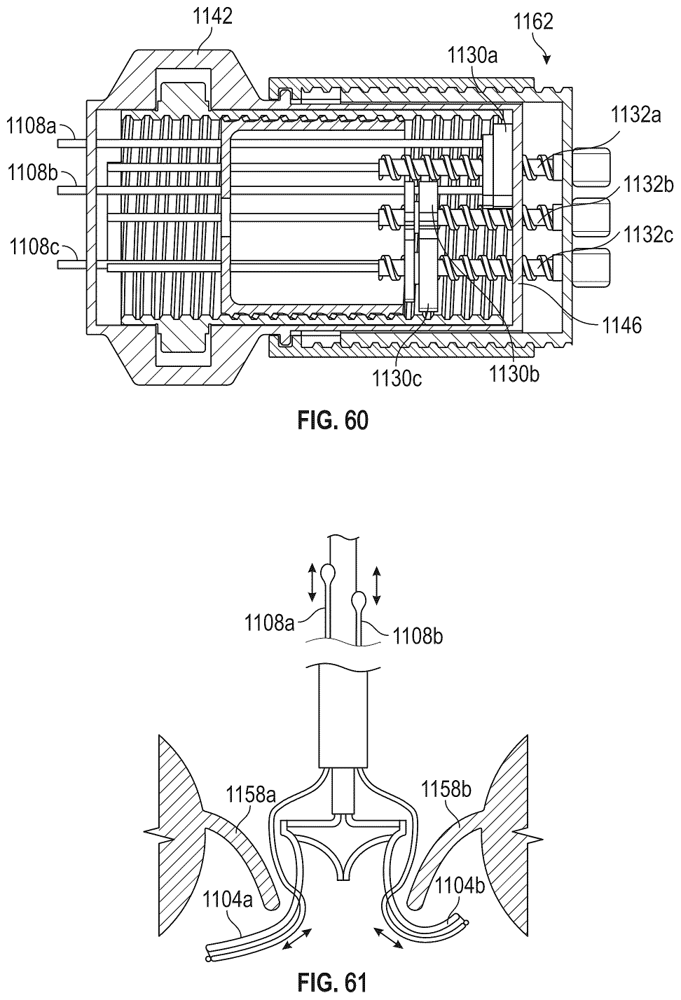

FIG. 60, for example, illustrates a position of the actuators 1132a, b, c upon operation of the offset controller 1162. The actuators 1132a, b, c may each be retracted to correspondingly retract the drive bodies 1130a, b, c and the tethers 1108a, b, c. The offset in position of the drive bodies 1130a, b, c and tethers 1108a, b, c may be maintained from the position shown in FIG. 59 for example. As such, referring to FIG. 61, the anchors 1104a, b may be deflected simultaneously with the offset shown in FIG. 59 maintained. Such an operation may allow for collective capture, recapture, or retraction of the anchors 1104a, b while maintaining an offset. The offset, for example, may account for variations in sizes of the leaflets 1158a, b.

Referring to FIG. 60, the offset controller 1162 may be operated until the proximal surface 1146 of the housing 1142 contacts one of the drive bodies 1130a. Such a feature may reduce the possibility of the offset controller 1162 undesirably varying the offset between the drive bodies 1130a, b, c and between the tethers 1108a, b, c.

In examples, the control mechanism 1102 may include an override mechanism 1173. The override mechanism 1173 may be configured to override a deflection offset between the anchors 1104a, b.

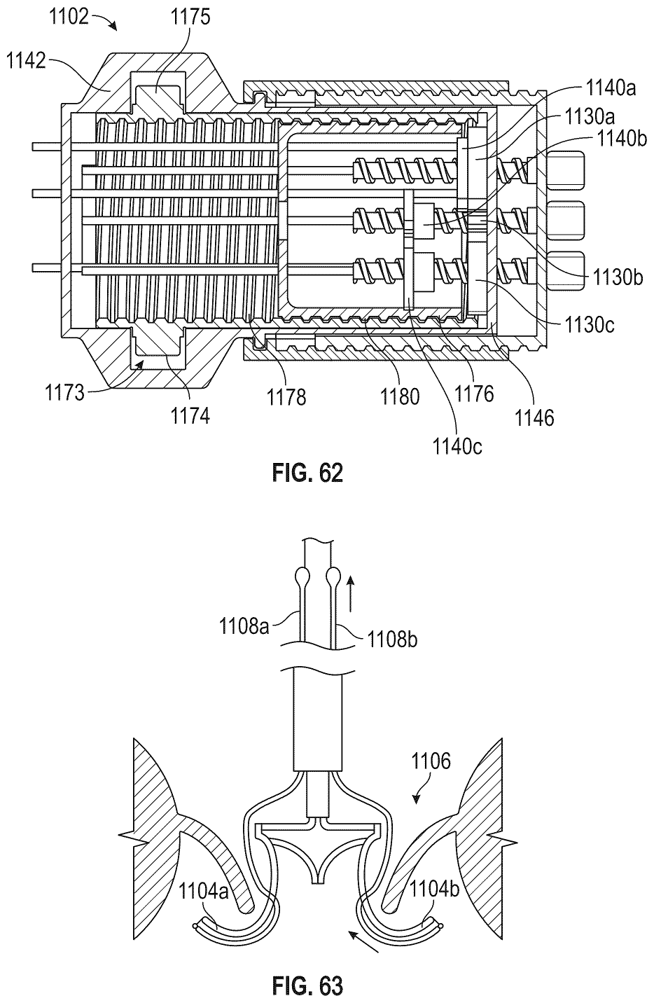

For example, referring to FIG. 62, the override mechanism 1173 may include an actuator 1175 for moving at least one of the drive bodies 1130a, b, c. The actuator 1175 may slide the drive bodies 1130a, b, c relative to each other to override the deflection offset that may exist between the drive bodies 1130a, b, c.

The actuator 1175 may include a control surface 1174 and a pressing body 1176. The control surface 1174 may be configured to be operated by a user to actuate the actuator 1175. The control surface 1174, for example, may comprise an outer grip surface upon the housing 1142. The control surface 1174 may be rotated. The control surface 1174 may include threading 1178 that may be configured to engage with threading 1180 on the pressing body 1176. The control surface 1174 may be coupled to the housing 1142 such that the axial position of the control surface 1174 does not vary upon rotation of the control surface 1174.

The pressing body 1176 may be positioned interior of the control surface 1174. The pressing body 1176 may be configured to slide axially along the housing 1142 upon rotation of the control surface 1174. The pressing body 1176 may include an outer surface having the threading 1180. As such, upon rotation of the control surface 1174, the pressing body 1176 may be moved axially proximal or distal.

The pressing body 1176 may be configured to contact the drive bodies 1130a, b, c. For example, a portion of the drive bodies 1130a, b, c may protrude from the outer profile of the respective adaptors 1140a, b, c to be pressed by the pressing body 1176 without the adaptors 1140a, b, c being pressed. As such, the drive bodies 1130a, b, c may move independent from the respective adaptors 1140a, b, c as shown in FIG. 62. The movement of the drive bodies 1130a, b, c may align the drive bodies 1130a, b, c such that an offset of the drive bodies 1130a, b, c and an offset of the tethers 1108a, b, c is overridden.

For example, FIG. 63 illustrates the tether 1108b being retracted proximally to a same position as the tether 1108a by the override mechanism 1173. Such a feature may allow a user to provide a similar deflection for all the anchors 1104a, b of the implant 1106. The override mechanism may move the tether 1108b a greater distance than the tether 1108a.

Referring to FIG. 62, the override mechanism 1173 may operate until the pressing body 1176 presses all the drive bodies 1130a, b, c against the proximal surface 1146 of the housing 1142. Such a feature may serve to confirm that the offset between the drive bodies 1130a, b, c and the offset between the tethers 1108a, b, c is overridden.

In examples, the actuators 1132a, b, c may be operated without rotation of the control surfaces 1136a, b, c. For example, referring to FIG. 58, the actuator 1132a may be operated by being pulled proximally through the opening 1156 (marked in FIG. 55) of the housing and the pressing body 1168. Such a feature may be accommodated by the opening 1156 (marked in FIG. 55) of the housing and the pressing body 1168 being unthreaded. As such, a user may manually and rapidly retract the tether 1108a to adjust the deflection of the anchor 1104a independently as desired. A tension in the tether 1108a may cause the actuator 1132a to be advanced distally upon release by the user.

Upon the anchors 1104a, b being in a desired position, the tethers may be cut or otherwise released from the implant 1106 to allow the implant to remain in an implanted position.

Other configurations of control mechanisms 1102 may be utilized. Features of the delivery system 1100 and control mechanism 1102 may be utilized solely or in combination with any example disclosed herein.

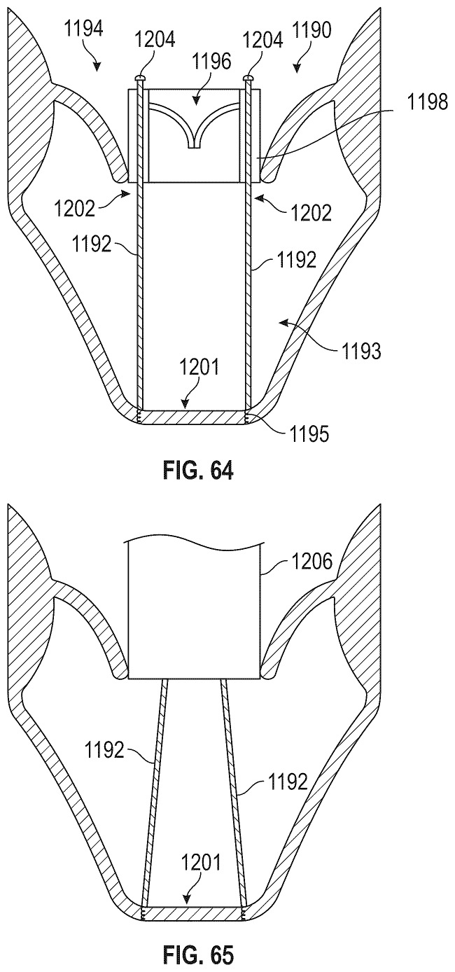

FIG. 64 illustrates an example of a prosthetic valve 1190 including one or more pacemaker leads 1192. The pacemaker leads 1192 may be configured to anchor the prosthetic valve 1190 in position at a native valve 1194.

The prosthetic valve 1190 may include a plurality of prosthetic valve leaflets 1196 and a valve body 1198 supporting the plurality of prosthetic valve leaflets 1196. The prosthetic valve 1190 may be configured to be deployed to the native valve 1194.

The pacemaker leads 1192 may be configured to anchor to an interior heart wall. The pacemaker leads 1192 may be configured to extend from the valve body 1198 to engage an interior surface 1201 of the interior heart wall. The interior heart wall, for example, may comprise a ventricle (e.g., the right ventricle when the valve 1194 is a tricuspid valve, and a left ventricle when the valve 1194 is a mitral valve). The interior surface 1201 may comprise a desired surface for implantation of the pacemaker leads 1192. For example, the interior surface 1201 may comprise a ventricular apex or another portion of a ventricle for implantation.

The pacemaker leads 1192 may include a distal portion 1193 having a tip 1195. The tip 1195 may be configured to penetrate the interior heart wall to anchor to the interior heart wall. Other forms of anchoring may be utilized in examples.

The pacemaker leads 1192 may extend transventricular to the valve body 1198. Proximal portions 1202 of the pacemaker leads 1192 may couple to the valve body 1198. The pacemaker leads 1192 may have sufficient strength to anchor the valve body 1198 in position against forces in an atrial direction. In examples, the pacemaker leads 1192 may have sufficient strength to anchor the valve body 1198 in position against forces in a ventricular direction.

The proximal portions 1202 of the pacemaker leads 1192 may include one or more electrical terminals 1204 for coupling with a pacemaker. The electrical terminals 1204 may be configured to electrically couple to the pacemaker. The pacemaker may be provided for a patient at a time of implantation of the prosthetic valve 1190, or at a later time.

In examples, the pacemaker leads 1192 may be deployed initially and the valve body 1198 may be deployed subsequently. FIG. 65, for example, illustrates a step in a deployment procedure in which the pacemaker leads 1192 may be initially deployed to the interior surface 1201 of the heart wall. The valve body 1198 may remain in an implant retention area of a delivery system such as a capsule 1206. The valve body 1198 may then be deployed from the capsule 1206 as desired. Other forms of implant retention areas may be utilized in examples.

The prosthetic valve 1190 may lack other anchors for anchoring to native leaflets or other portions of the native valve in examples. As such, a reduced possibility of missed capture or tangle with native valve leaflets or chordae may result. A reduced possibility of cutting of chordae may result. Conduction disturbance provided by such anchors may also be reduced. In examples, the prosthetic valve 1190 may include anchors for anchoring to native leaflets or other portions of the native valve, as may be disclosed herein.

The pacemaker leads 1192 may be configured to have an adjustable length. For example, the pacemaker leads 1192 may be configured to slide relative to the valve body 1198. Upon deployment of the valve body 1198, the delivery system may be utilized to tension the pacemaker leads 1192 relative to the valve body 1198. The length of the pacemaker lead 1192 may be adjusted in vivo to account for a varied size of the patient's anatomy.

The pacemaker leads 1192 may be configured to be positioned between inner and outer valve bodies in examples. The pacemaker leads 1192 may be configured to be positioned between inner and outer frames in examples. The configuration of a valve body may be similar to other configurations of valve bodies disclosed herein. Other forms of valve bodies may be utilized. Other positions of the pacemaker leads 1192 may be utilized in examples.

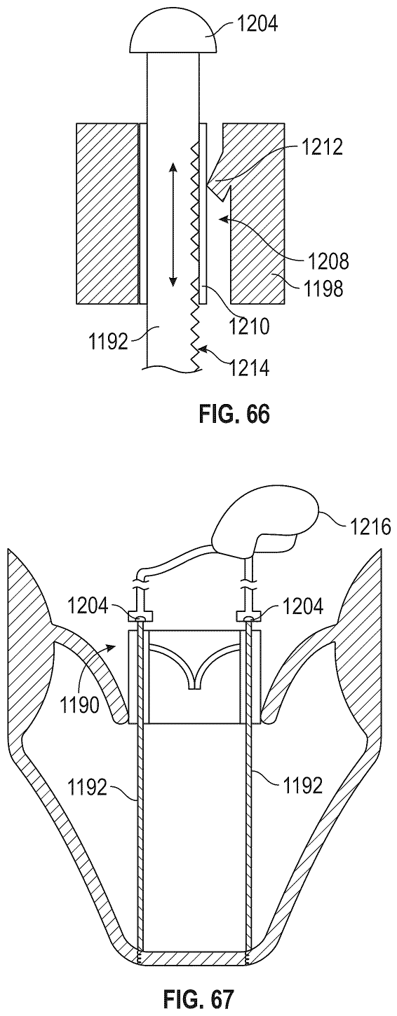

A lock may be utilized for locking the pacemaker lead 1192 in position relative to the valve body 1198. The lock may be activated during deployment and positioning. A length of each pacemaker lead 1192 may be determined. For example, referring to FIG. 66, the lock 1208 may be engaged upon the length of the pacemaker lead 1192 being determined. The lock 1208 may be positioned on a proximal or atrial side of the prosthetic valve in examples. Other positions may be utilized. A lock release 1210 or other mechanism may be utilized to allow the lock 1208 to engage. The lock release 1210 may be activated or removed to allow the lock 1208 to engage. For example, a lock release 1210 in the form of a spacer is shown in FIG. 66. The spacer may be removed to allow the lock to lock in position. The lock 1208 may have a variety of forms. For example, a lock may comprise a protrusion 1212 configured to contact an engagement surface 1214 in examples. Other forms of locks may be utilized as desired (either electrical or mechanical for example).

Upon deployment of the prosthetic valve 1190, a pacemaker 1216 may be provided for the patient. Referring to FIG. 67, the pacemaker 1216 may be provided and coupled to the electrical terminals 1204 of the prosthetic valve 1190. In examples, the pacemaker 1216 may be deployed in a later procedure and may be connected to the electrical terminals 1204 at that time. In examples, the pacemaker 1216 may be provided during the implantation procedure of the prosthetic valve 1190. In examples, the pacemaker 1216 may be connected to the pacemaker leads 1192 and implanted at the same time as the pacemaker leads 1192.

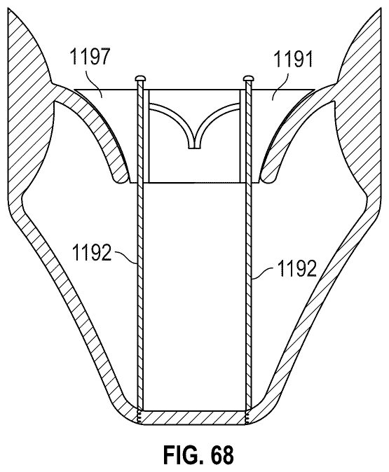

Other configurations of prosthetic valves and pacemaker leads may be utilized. FIG. 68, for example, illustrates a variation of the prosthetic valve 1190 in which the valve body 1191 includes an atrial flange 1197. The atrial flange 1197 may comprise an anchor for resisting ventricular movement of the prosthetic valve. The prosthetic valve may otherwise include the features stated in regard to the prosthetic valve 1190. Features as described in regard to FIGS. 64-68 may be utilized solely or in combination with any example disclosed herein.

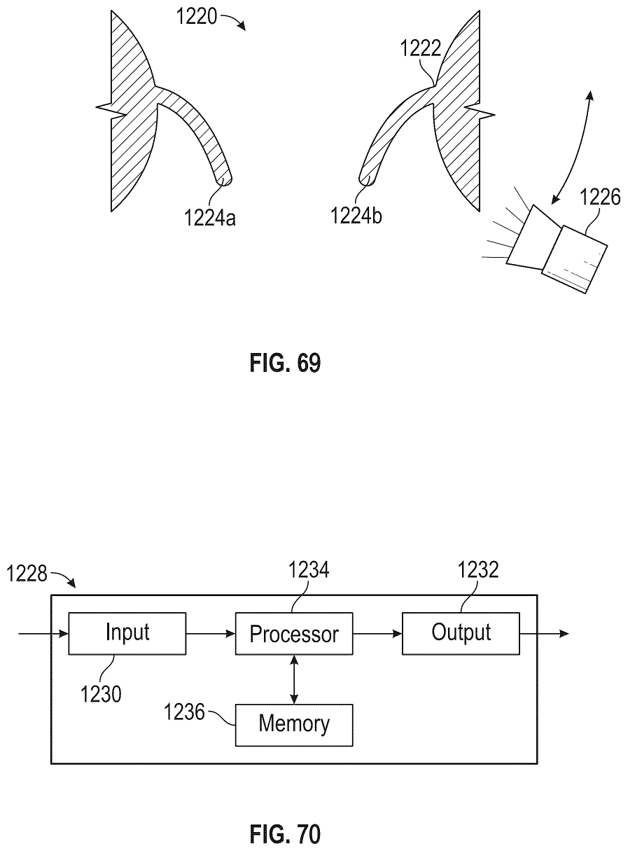

Apparatuses, systems, and methods disclosed herein may be directed to manufacturing at least a portion of a prosthetic heart valve based on an imaging of a native heart valve. FIGS. 69-77 illustrate exemplary apparatuses, systems, and methods that may be utilized, although such apparatuses, systems, and methods are not so limited.

Referring to FIG. 69, for example, a native heart valve 1220 may be imaged. The imaging may have a variety of forms. The imaging may comprise x-ray imaging (e.g., a computerized tomography (CT) scan) or other forms of x-ray imaging. The imaging may comprise ultrasound imaging (e.g., cchocardiograph imaging). In examples, combinations of forms of imaging or other forms of imaging may be utilized.

The imaging may be utilized to determine a shape of the native heart valve 1220. The shape of the native heart valve 1220 may comprise a shape of the annulus 1222 of the native heart valve 1220. The shape of the native heart valve 1220 may comprise a length or position of the native valve leaflets 1224a, b. Other features of the native heart valve 1220 may be determined with the imaging.

The imaging may occur with an imaging apparatus 1226 (e.g., an x-ray or ultrasound scanning device) that may provide a full scan of the native heart valve 1220, or a partial scan as desired. The imaging apparatus 1226 may be moved to a variety of positions to provide a desired scan of the native heart valve 1220 in examples. A three dimensional scan or model of the native heart valve 1220 may be provided, although other forms of scanning (e.g., two dimensional scan or model) may be provided as desired.

The imaging may determine an irregular shape of the native heart valve 1220 (e.g., an irregular shaped annulus 1222) or other features of the native anatomy as desired.

In examples, the imaging may occur prior to a procedure for deploying the prosthetic heart valve. For example, the imaging may occur as a separate procedure, which may occur days or weeks prior to the implantation procedure of the prosthetic heart valve. The imaging may occur for the purpose of determining the shape of the native heart valve 1220 for manufacture of at least a portion of the prosthetic heart valve.

The image of the native heart valve 1220 produced during the imaging procedure may be stored or transmitted to a processing system. FIG. 70, for example, illustrates an exemplary processing system 1228. The processing system 1228 may include an input 1230, an output 1232, a processor 1234, and a memory 1236. Other configurations of processing systems 1228 may be utilized in examples.

The input 1230 may have a variety of forms and may comprise a data port, a wireless transceiver, or an input terminal, among other forms of inputs. The input 1230 may be configured to receive signals or data for use by the processing system 1228.

The output 1232 may comprise a data port, a wireless transceiver, or an output terminal, among other forms of outputs. The output 1232 may be configured to output signals or data provided by the processing system 1228.

The memory 1236 may be configured to store data (e.g., programs, instructions, parameters, etc.) for processing by the processor 1234. The memory 1236 may comprise non-transitory memory for storing such data for use by the processor 1234. The memory 1236 may comprise a hard drive (e.g, mechanical or solid state), flash memory, or random access memory (RAM), among other forms of memory.

The processor 1234 may be configured to perform the processes disclosed herein. The processor 1234 may have a variety of forms and may comprise a microprocessor, a controller, a distributed processing network, among other forms of processors. In examples, components of the processing system 1228 may be remote and may operate utilizing wired or wireless transmissions, the internet, or may comprise a cloud computing environment.

The processor 1234 may be configured to operate based on data received from the memory 1236 or otherwise provided to the processor 1234. The processor 1234 may receive data from the input 1230 for processing, to be provided to the output 1232. The processor 1234 may perform some or all of the processes disclosed herein.

In examples, the processor 1234 may receive image data from the input 1230 that may be produced during the imaging procedure represented in FIG. 69. The image data may comprise a model of the native heart valve 1220 or may comprise data allowing the processor 1234 to form a model of the native heart valve 1220. In examples, the image data may be in a format for the processor 1234 to produce an output 1232 of one or more features of a prosthetic heart valve based on the image data produced during the imaging procedure. The image data, for example, may comprise parameters of a shape of the native heart valve 1220 for use by the processor 1234.

The processor 1234 may be configured to produce an output 1232 for use in manufacture of at least a portion of the prosthetic heart valve. The output 1232, for example, may comprise a shape or other configuration of at least a portion of the prosthetic heart valve, or may comprise a shape or other configuration of a tool utilized to form at least a portion of the prosthetic heart valve. Other forms of output 1232 may be provided.

In examples, the processor 1234 may be configured to qualify a determined shape against one or more limits. For example, a fatigue or crimp strain limit for a frame may be evaluated by the processor 1234 to qualify the frame against. The limits may be previously determined and stored in the memory 1236 and provided to the processor 1234. The processor 1234 may determine such a shape and produce a shape that meets the one or more limits. Finite element analysis may be utilized by the processor 1234. The processor 1234 may be configured to determine whether at least a portion of the prosthetic heart valve complies with limits such as strain limits.

In examples, the processor 1234 may utilize one or more artificial intelligence algorithms to determine an optimized shape of the portion of the prosthetic valve. The artificial intelligence algorithms may be utilized to determine if the shape (of a portion of a prosthetic valve or a tool) is qualified against one or more limits. The artificial intelligence algorithms may utilize the image data to produce a portion of a prosthetic valve or a tool based on the image data. Other inputs may be provided to the artificial intelligence algorithms (e.g., prior data from other prosthetic valves or tools, or other parameters input by a user into the artificial intelligence algorithms). In examples, artificial intelligence algorithms may not be utilized.

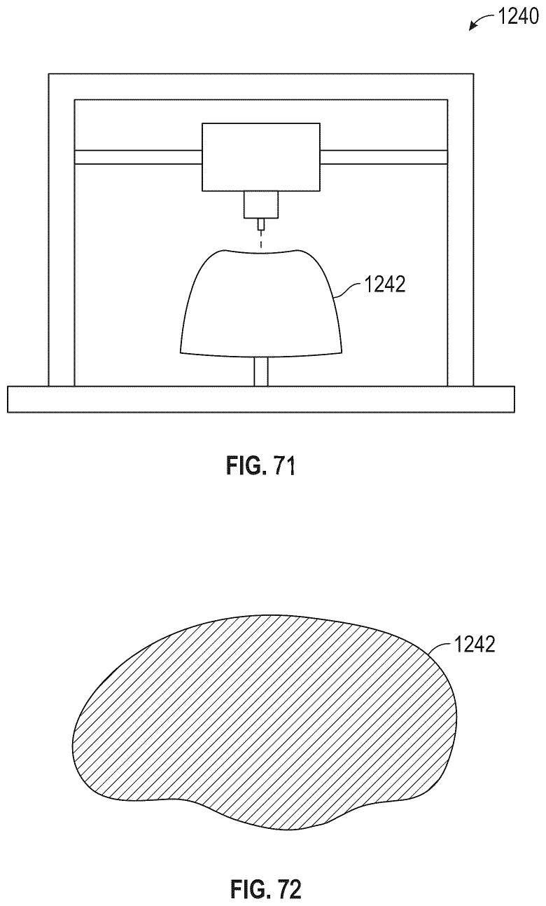

The processing system 1228 may comprise a portion of a fabrication assembly 1240 that may be utilized to fabricate at least a portion of the prosthetic heart valve, or at least a portion of a tool utilized to form at least a portion of the prosthetic heart valve. FIG. 71, for example, illustrates an exemplary fabrication assembly 1240. The fabrication assembly 1240 may be utilized to shape, form, or generate at least a portion of the prosthetic heart valve or a tool utilized to form at least a portion of the prosthetic heart valve. The fabrication assembly 1240 may comprise an automated fabrication assembly in examples. For example, an input may be provided to the fabrication assembly 1240 for the assembly 1240 to perform the fabrication in an automated manner. The input may be provided from the output 1232 of the processing system 1228.

In an exemplary procedure, the fabrication assembly 1240 may be configured to form a tool. The fabrication assembly 1240, for example, may shape, form, or generate the tool. The tool may comprise a mandrel 1242 as shown in FIG. 71. The mandrel 1242 may be configured for at least a portion of a valve body (e.g., a frame of a valve body) to be formed upon.

In examples, the mandrel 1242 may be formed by the fabrication assembly 1240 utilizing additive manufacturing based on the imaging of the native heart valve. Three-dimensional printing or other forms of additive manufacturing may be utilized to form the shape of the mandrel 1242. FIG. 71, for example, illustrates the mandrel 1242 being formed with an additive manufacturing process.

The mandrel 1242 may be formed in a shape based on the output provided by the processor 1234. For example, the processor 1234 may determine a desired shape of the mandrel 1242 based on the image data provided from the imaging procedure. The prosthetic heart valve may include a valve body. The valve body may be configured to support a plurality of prosthetic valve leaflets. The shape of the mandrel 1242 may be determined to produce the valve body based on the shape of the native heart valve 1220.

The shape of the mandrel 1242 may be determined based on the imaged shape of the annulus 1222 of the native heart valve 1220 for example. The mandrel 1242 may be utilized to provide a shape of at least a portion of the valve body based on the imaged shape of the native heart valve. The mandrel 1242 may be shaped to produce a valve body that conforms to the shape of the annulus 1222 in examples. The mandrel 1242 may be formed, shaped, or generated to produce such a shape for the valve body to be formed upon.

The shape may be non-circular in examples. For example, FIG. 72 illustrates a top cross-sectional view of the generated mandrel 1242, showing the outer profile of the mandrel 1242. The outer profile comprises a non-circular shape. The outer profile comprises an irregular shape. For example, the outer profile is a non-uniform ovoid shape. The outer profile may conform to a non-circular and irregular shape of the native heart valve 1220.

The mandrel 1242 may be configured for at least a portion of the valve body to be formed upon, to match the shape of the mandrel 1242.

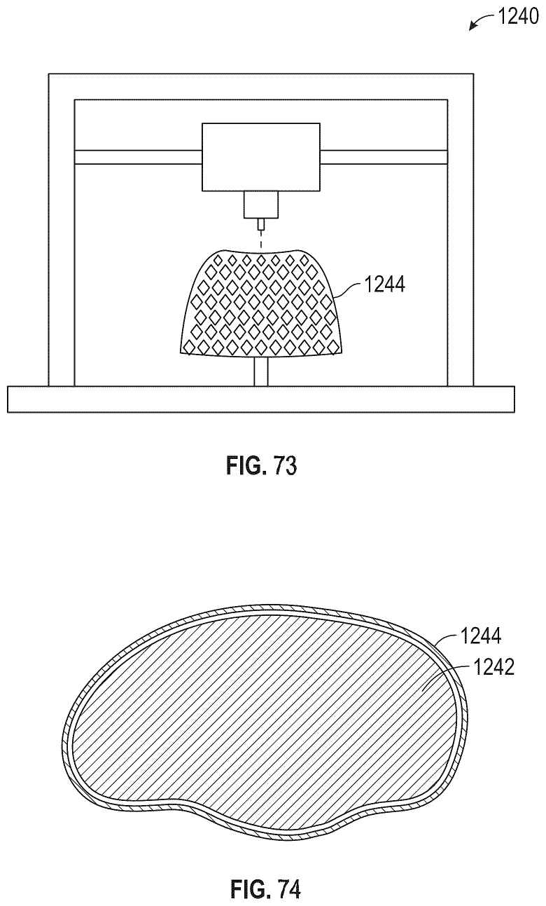

A valve body may be manufactured according to methods disclosed herein. At least a portion of the valve body may be manufactured based on the imaging of the native heart valve. For example, referring to FIG. 73, the fabrication assembly 1240 may be utilized to form, shape, or generate at least a portion of a valve body. The fabrication assembly 1240, for example, may form the valve body 1244 utilizing additive manufacturing. The shape or other configuration of the valve body 1244 may be provided based on the output provided by the processor 1234. For example, the processor 1234 may determine a desired shape of at least a portion of the prosthetic heart valve based on the imaging of the native heart valve.

The processor 1234 may determine the portion of the valve body 1244 based on the image data provided from the imaging procedure. An optimized shape may be determined. The shape of the valve body 1244 may be determined to produce a valve body based on the imaged shape of the native heart valve 1220. The shape of the valve body 1244 may be determined based on the shape of the annulus 1222 of the native heart valve 1220 for example. The valve body 1244 may be shaped to conform to the shape of the annulus 1222 in examples.

The valve body 1244 may have a non-circular or irregular outer profile in examples. Other configurations of valve bodies may be provided in examples.

In examples, the portion of the valve body 1244 may comprise a frame of the valve body. At least a portion of the frame may be manufactured. In examples, the shape of the frame may be determined based on the output provided by the processor 1234. A shape of at least a portion of the frame may be provided based on the imaged shape of the native heart valve. Other features of the frame may be determined based on the output provided by the processor 1234. For example, a cut pattern of the frame may be determined based on the output provided by the processor 1234. The fabrication assembly 1240 may be configured to cut material for the frame (e.g., a laser cut or other form of cutting) based on the output provided by the processor 1234. FIG. 73, for example, illustrates the fabrication assembly 1240 cutting material for the frame in a desired shape.

In examples, the valve body 1244 may be shaped upon the mandrel 1242 that may be formed. FIG. 74, for example, illustrates the valve body 1244 upon the mandrel 1242, and matching the shape provided by the mandrel 1242. The valve body 1244 may be heat set upon the mandrel 1242. In examples, the use of the mandrel 1242 may be excluded and the fabrication assembly 1240 may form the portion of the valve body 1244 directly without shaping upon the mandrel 1242.

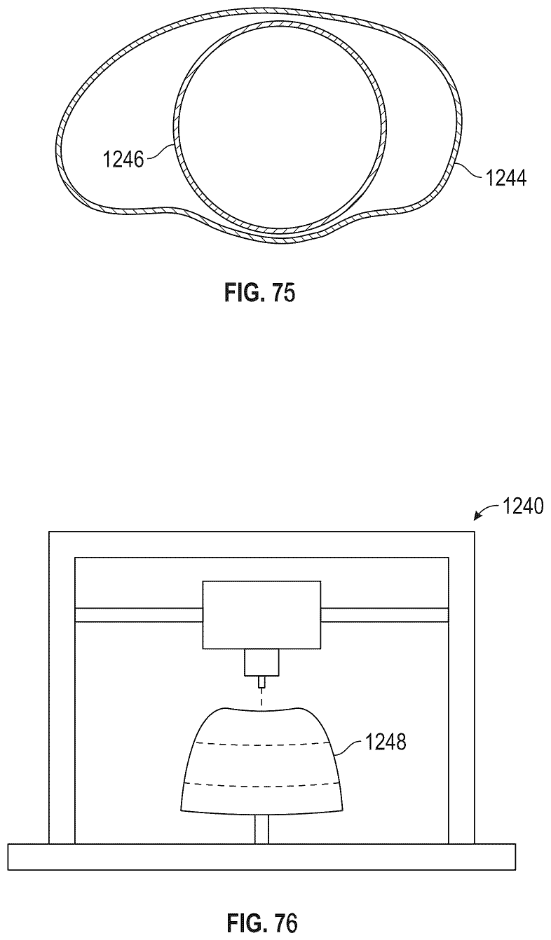

The portion of the valve body 1244 being shaped may comprise an outer valve body in examples. For example, referring to FIG. 75, the outer valve body 1244 may be positioned radially outward of an inner valve body 1246. At least a portion of the outer valve body 1244 may be formed. The outer valve body 1244 may retain the shape determined by the processor 1234. The inner valve body 1246, however, may comprise a different shape and may comprise a circular outer profile or other standard or uniform shape as desired. As such, the outer valve body 1244 may be contoured to a shape of the native heart valve and the inner valve body 1246 may comprise a standard or uniform shape. The inner valve body 1246 accordingly may better support prosthetic valve leaflets and provide a symmetrical flow channel for the native heart valve. The inner valve body 1246 for example may comprise a standard non-custom shape that has been previously qualified to meet fatigue and strain limits, for example.

The outer valve body 1244 may comprise a scaling body for forming a seal with the native heart valve. The outer valve body 1244 may comprise a scaling skirt or may comprise an outer sealing frame or may have another configuration as desired. In examples, a prosthetic valve manufactured may comprise a single frame or frame lacking both an outer frame and an inner frame.

The processing system 1228 may determine if a portion of the prosthetic valve to be manufactured will not meet one or more limits (e.g., strain limits, or fatigue limits). As such, the processing system 1228 may adjust the determined shape of the prosthetic valve or tool to meet the one or more limits.

Any portion of the frame may be self-expanding. In examples, a portion of a frame may be balloon expandable or mechanically expandable as desired.

In examples, the portion of the valve body 1244 may comprise a skirt of the valve body. At least a portion of the skirt may be manufactured based on the imaging of the native heart valve. In examples, the shape of the skirt may be determined based on the output provided by the processor 1234. Other features of the skirt may be determined based on the output provided by the processor 1234. For example, a stitching pattern or suture pattern of the skirt may be determined based on the output provided by the processor 1234. Referring to FIG. 76, the fabrication assembly 1240 may be configured to cut material for the skirt 1248 (e.g., a laser cut or other form of cutting) based on the output provided by the processor 1234. The fabrication assembly may be configured to produce a stitching pattern or suture pattern for the skirt. FIG. 76, for example, illustrates the fabrication assembly 1240 providing a stitching pattern or suture pattern on the skirt 1248.

In examples, the configuration of the skirt may be determined based on the determined configuration of the valve body 1244. For example, the stitching or suture pattern of the skirt may be determined to accommodate the shape of a frame of the valve body 1244.

In examples, a frame of the valve body 1244 may be covered utilizing an electrospinning technology.

One or more features of the prosthetic valve leaflets may be manufactured according to methods disclosed herein. For example, a processor 1234 may determine a desired shape of prosthetic valve leaflets to be utilized with a shape of a valve body 1244. The fabrication assembly 1240 may be utilized to cut or otherwise form one or more of the prosthetic valve leaflets.

The fabrication assembly 1240 may assemble the components of the prosthetic valve in examples. For example, an automated assembly process may be utilized to form the prosthetic valve. The assembly process may be autonomous in examples. A fabrication assembly, for example, may receive an input and entirely manufacture a prosthetic valve based on the input. An autonomous assembly line process may be utilized. In examples, semi-autonomous processes (e.g., with portions of manual assembly) may be utilized.

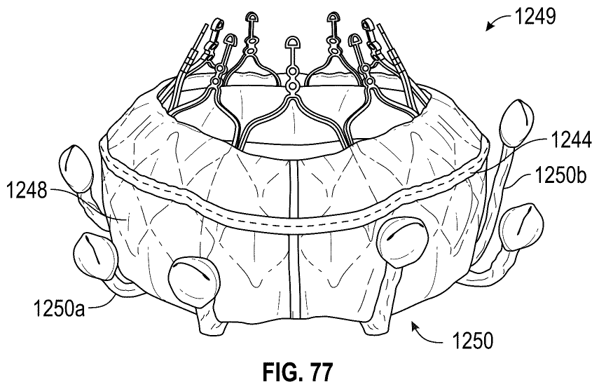

A resulting configuration of a prosthetic valve 1249 may be represented in FIG. 77. Further, a configuration of one or more anchors 1250 may be manufactured according to methods disclosed herein. The configuration of the anchors 1250 may be provided based on the imaging of the native heart valve. For example, a quantity, position, and size of anchors may be manufactured based on the output provided by the processor 1234. The processor 1234, for example, may determine based on image data that an anchor 1250b should have a greater length than the anchor 1250a based on the shape of the native valve (e.g., a length of a native valve leaflet or other configuration of the native valve). The processor 1234, for example, may determine that a position of an anchor should be provided to reduce conduction disturbance to the native heart valve and should be shorter or be positioned in a particular location. The processor 1234 may determine a number of anchors 1250 that should be utilized. The anchors 1250 may comprise distal anchors configured similarly as other forms of distal anchors disclosed herein. In examples, the anchors may comprise other forms of anchors.

The prosthetic valve 1249 produced may be manufactured on a custom basis for the particular heart valve that is imaged. As such, a patient may receive a heart valve that is custom shaped for the particular anatomy of that patient. Customization may provide for enhanced sealing, anchoring, and other functioning at the implantation site. At least a portion of the prosthetic valve 1249 may be manufactured to conform to a shape of the native heart valve. Improved scaling, anchoring, and functioning may occur at implantation sites having non-circular or irregular shapes. A custom fit for the native valve may be provided.

Automated manufacturing processes may increase the speed and efficiency at which the prosthetic valve may be manufactured. In-line rapid prototyping or other forms of high-speed manufacturing processes may be utilized. Rapid manufacture, testing, validation, and shipping may be provided. Artificial intelligence may be utilized. The prosthetic valve may be sterilized and shipped for implantation in the particular patient that has been imaged.

The prosthetic valve 1249 may be designed to be deployed for the particular patient that has been imaged.

The features of FIGS. 69-77 may be utilized solely or in combination with any example disclosed herein.

Although many of the systems and methods disclosed herein have been discussed in regard to implantation of a prosthetic valve implant, it is understood that the systems and methods may be utilized to deliver a variety of implants, including implants for repair of a heart valve. For example, other types of heart valve implants that may be utilized than are shown herein, among other types of implants (e.g., mitral, tricuspid, pulmonary, and aortic valve implants and other repair implants). Any of the implants or valves disclosed herein may comprise valves for implantation at a tricuspid or mitral valve, as well as aortic or pulmonary valves. Other implantation locations may be utilized in examples.

The methods and systems disclosed herein may in certain examples not be limited to delivery of implants, but may extend to any medical intervention or insertion into a patient's body, which may include performing a medical procedure within the body. The methods and systems disclosed herein may be utilized in general use of a catheter as desired. For example, the handle shown herein and components disclosed therein may comprise a general catheter handle in certain examples. Further, the configuration of the delivery apparatus may be modified in other examples. For example, for an aortic valve delivery apparatus, the configuration of the implant retention area and other features of the delivery apparatus may be modified. Delivery apparatuses as disclosed herein may comprise delivery catheters in examples.

The deflection mechanisms and other examples disclosed herein may be utilized for a variety of implementations including delivery of tricuspid or mitral replacement valves, or aortic or pulmonary valves, or for valve repair procedures, including tricuspid or mitral valve repair or aortic or pulmonary valve repair.

From the foregoing description, it will be appreciated that an inventive product and approaches for implant delivery systems are disclosed. While several components, techniques and aspects have been described with a certain degree of particularity, it is manifest that many changes can be made in the specific designs, constructions and methodology herein above described without departing from the spirit and scope of this disclosure.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, along and in various combinations and sub-combinations with one another. The methods, apparatuses, and systems are not limited to any specific aspect or feature or combination thereof, nor do the disclosed examples require that any one or more specific advantages be present or problems be solved. Features, elements, or combinations of one example can be combined into other examples herein.

Example 1: A delivery system for an implant, the delivery system comprising: an elongate shaft having: a distal end, an implant retention area for retaining the implant, a first bend portion configured to deflect the distal end in a first plane, a first extension portion positioned proximal of the first bend portion and extending along a first axis, a second bend portion positioned proximal of the first extension portion and configured to rotate in a first rotational direction in a second plane extending transverse to the first plane, a third bend portion positioned proximal of the second bend portion and configured to rotate in a second rotational direction that is opposite the first rotational direction in the second plane, and a second extension portion positioned proximal of the third bend portion and extending along a second axis; and a deflection mechanism configured to deflect the second bend portion in the first rotational direction in the second plane and deflect the third bend portion in the second rotational direction in the second plane to offset the first axis from the second axis with the first axis extending parallel with the second axis.

Example 2: The delivery system of any example herein, in particular Example 1, wherein the implant retention area is positioned distal of the first bend portion.

Example 3: The delivery system of any example herein, in particular Example 1 or Example 2, wherein the implant retention area includes a capsule configured to retract for deployment of the implant.

Example 4: The delivery system of any example herein, in particular Examples 1-3, wherein the distal end includes a nose conc.

Example 5: The delivery system of any example herein, in particular Examples 1-4, wherein the first bend portion is configured to deflect the distal end to extend transverse to the first axis.

Example 6: The delivery system of any example herein, in particular Examples 1-5, wherein the first bend portion is configured to deflect the distal end to extend perpendicular to the first axis.

Example 7: The delivery system of any example herein, in particular Examples 1-6, wherein the first extension portion and the second extension portion are configured to extend with the first axis coaxial with the second axis.

Example 8: The delivery system of any example herein, in particular Examples 1-7, further comprising a third extension portion positioned between the second bend portion and the third bend portion and configured to extend transverse to the first axis and the second axis.

Example 9: The delivery system of any example herein, in particular Examples 1-8, wherein the deflection mechanism is configured to deflect the second bend portion in the first rotational direction in the second plane and deflect the third bend portion in the second rotational direction in the second plane simultaneously.

Example 10: The delivery system of any example herein, in particular Examples 1-9, wherein the deflection mechanism is configured to deflect the second bend portion in the first rotational direction in the second plane a same amount as a deflection of the third bend portion in the second rotational direction in the second plane.

Example 11: The delivery system of any example herein, in particular Examples 1-10, wherein the deflection mechanism includes at least one pull tether configured to deflect the second bend portion and the third bend portion.

Example 12: The delivery system of any example herein, in particular Examples 1-11, wherein the deflection mechanism includes at least one pull tether coupled to the elongate shaft distal of the second bend portion and configured to deflect the second bend portion and the third bend portion.

Example 13: The delivery system of any example herein, in particular Examples 1-12, wherein the deflection mechanism includes a first pull tether coupled to the elongate shaft and configured to deflect the second bend portion in the first rotational direction in the second plane, and includes a second pull tether coupled to the elongate shaft and configured to deflect the third bend portion in the second rotational direction in the second plane.

Example 14: The delivery system of any example herein, in particular Example 13, wherein the first pull tether is coupled to a first portion of the elongate shaft and the second pull tether is coupled to a second portion of the elongate shaft that is opposed to the first portion.

Example 15: The delivery system of any example herein, in particular Example 14, wherein the first pull tether extends parallel with the second pull tether.

Example 16: The delivery system of any example herein, in particular Examples 13-15, wherein the first pull tether and the second pull tether each include a proximal portion coupled to a first pull body configured to retract the first pull tether and the second pull tether simultaneously.

Example 17: The delivery system of any example herein, in particular Example 16, further comprising a handle at a proximal end of the elongate shaft, wherein the first pull body is configured to be retracted within the handle to retract the first pull tether and the second pull tether simultaneously.

Example 18: The delivery system of any example herein, in particular Examples 11-17, further comprising at least one pull tether configured to deflect the distal end in the first plane.

Example 19: The delivery system of any example herein, in particular Examples 1-18, wherein the second bend portion includes at least one flex cut configured to allow the second bend portion to deflect, and the third bend portion includes at least one flex cut configured to allow the third bend portion to deflect.

Example 20: The delivery system of any example herein, in particular Examples 1-19, wherein the first bend portion includes at least one flex cut configured to allow the first bend portion to deflect.

Example 21: A method comprising: delivering a delivery apparatus for an implant into a portion of a patient's body, the delivery apparatus including: an elongate shaft having: a distal end, an implant retention area retaining the implant, a first bend portion configured to deflect the distal end in a first plane, a first extension portion positioned proximal of the first bend portion and extending along a first axis, a second bend portion positioned proximal of the first extension portion and configured to rotate in a first rotational direction in a second plane extending transverse to the first plane, a third bend portion positioned proximal of the second bend portion and configured to rotate in a second rotational direction that is opposite the first rotational direction in the second plane, and a second extension portion positioned proximal of the third bend portion and extending along a second axis; and a deflection mechanism configured to deflect the second bend portion in the first rotational direction in the second plane and deflect the third bend portion in the second rotational direction in the second plane to offset the first axis from the second axis with the first axis extending parallel with the second axis.

Example 22: The method of any example herein, in particular Example 21, wherein the implant retention area includes a capsule configured to retract for deployment of the implant.

Example 23: The method of any example herein, in particular Example 21 or Example 22, wherein the first bend portion is configured to deflect the distal end to extend perpendicular to the first axis.

Example 24: The method of any example herein, in particular Examples 21-23, wherein the first extension portion and the second extension portion are configured to extend with the first axis coaxial with the second axis.

Example 25: The method of any example herein, in particular Examples 21-24, further comprising a third extension portion positioned between the second bend portion and the third bend portion and configured to extend transverse to the first axis and the second axis.

Example 26: The method of any example herein, in particular Examples 21-25, wherein the deflection mechanism is configured to deflect the second bend portion in the first rotational direction in the second plane and deflect the third bend portion in the second rotational direction in the second plane simultaneously.

Example 27: The method of any example herein, in particular Examples 21-26, wherein the deflection mechanism is configured to deflect the second bend portion in the first rotational direction in the second plane a same amount as a deflection of the third bend portion in the second rotational direction in the second plane.

Example 28: The method of any example herein, in particular Examples 21-27, wherein the deflection mechanism includes at least one pull tether configured to deflect the second bend portion and the third bend portion.

Example 29: The method of any example herein, in particular Examples 21-28, further comprising delivering the implant to a native mitral heart valve or a native tricuspid heart valve.

Example 30: The method of any example herein, in particular Examples 21-29, further comprising deflecting the second bend portion in the first rotational direction in the second plane and deflecting the third bend portion in the second rotational direction in the second plane within a heart atrium.

Example 31: A delivery system for an implant, the delivery system comprising: an elongate shaft having: a retention body configured to retain the implant; and a diaphragm extending proximally and configured to be moved distally to allow the retention body to release from the implant.

Example 32: The delivery system of any example herein, in particular Example 31, wherein the diaphragm is configured to invert upon being moved distally.

Example 33: The delivery system of any example herein, in particular Example 31 or Example 32, wherein the diaphragm has a narrow portion and a wide portion, with the wide portion extending proximally from the narrow portion.

Example 34: The delivery system of any example herein, in particular Example 33, wherein the diaphragm is configured to invert with the wide portion extending distally from the narrow portion.

Example 35: The delivery system of any example herein, in particular Examples 31-34, wherein the diaphragm has a conical shape.

Example 36: The delivery system of any example herein, in particular Examples 31-35, wherein the diaphragm has an interior cavity and an inner surface facing radially inward and facing the interior cavity, and wherein the diaphragm is configured to invert with the inner surface facing radially outward.

Example 37: The delivery system of any example herein, in particular Examples 31-36, wherein the diaphragm includes a distal end portion and the retention body includes a proximal end portion coupled to the distal end portion of the diaphragm.

Example 38: The delivery system of any example herein, in particular Examples 31-37, wherein the retention body is configured to retract proximally to release from the implant.

Example 39: The delivery system of any example herein, in particular Example 38, wherein the retention body includes an outer surface and the diaphragm is configured to invert upon being moved distally to overlay the outer surface of the retention body.

Example 40: The delivery system of any example herein, in particular Examples 31-39, wherein the retention body covers a coupling body configured to couple to the implant, and the retention body is configured to retract from the coupling body upon the diaphragm being moved distally.

Example 41: The delivery system of any example herein, in particular Examples 31-40, wherein the diaphragm includes a proximal end and the retention body includes a distal end, and a length from the proximal end of the diaphragm to the distal end of the retention body is configured to reduce upon the diaphragm being moved distally.

Example 42: The delivery system of any example herein, in particular Examples 31-41, wherein the elongate shaft includes an inner shaft, and the retention body and the diaphragm are configured to slide proximally along the inner shaft.

Example 43: The delivery system of any example herein, in particular Examples 31-42, wherein the elongate shaft includes a pusher shaft configured to be advanced distally to move the diaphragm distally.

Example 44: The delivery system of any example herein, in particular Example 43, wherein the diaphragm includes a proximal end portion, and the pusher shaft is configured to apply a force to the proximal end portion of the diaphragm.

Example 45: The delivery system of any example herein, in particular Examples 31-44, further comprising the implant, wherein the implant comprises a self expanding prosthetic heart valve.

Example 46: A method comprising: delivering a delivery apparatus for an implant into a portion of a patient's body, the delivery apparatus including: an elongate shaft having: a retention body retaining the implant, and a diaphragm extending proximally and configured to be moved distally to allow the retention body to release from the implant.

Example 47: The method of any example herein, in particular Example 46, wherein the diaphragm is configured to invert upon being moved distally.

Example 48: The method of any example herein, in particular Example 46 or Example 47, wherein the diaphragm has a narrow portion and a wide portion, with the wide portion extending proximally from the narrow portion.

Example 49: The method of any example herein, in particular Example 48, wherein the diaphragm is configured to invert with the wide portion extending distally from the narrow portion.

Example 50: The method of any example herein, in particular Examples 46-49, wherein the diaphragm has an interior cavity and an inner surface facing radially inward and facing the interior cavity, and wherein the diaphragm is configured to invert with the inner surface facing radially outward.

Example 51: The method of any example herein, in particular Examples 46-50, wherein the retention body is configured to retract proximally to release from the implant.

Example 52: The method of any example herein, in particular Example 51, wherein the retention body includes an outer surface and the diaphragm is configured to invert upon being moved distally to overlay the outer surface of the retention body.

Example 53: The method of any example herein, in particular Examples 51-52, wherein the diaphragm includes a proximal end and the retention body includes a distal end, and a length from the proximal end of the diaphragm to the distal end of the retention body is configured to reduce upon the diaphragm being moved distally.

Example 54: The method of any example herein, in particular Examples 51-53, wherein the elongate shaft includes a pusher shaft configured to be advanced distally to move the diaphragm distally.

Example 55: The method of any example herein, in particular Examples 51-54, wherein the implant comprises a self expanding prosthetic heart valve.

Example 56: A delivery system for an implant, the delivery system comprising: one or more suction ports configured to apply suction to native heart valve leaflets of a native heart valve to draw the native heart valve leaflets radially inward; and an elongate shaft having an implant retention area and configured to at least partially deploy the implant from the implant retention area to the native heart valve with the one or more suction ports applying the suction to the native heart valve leaflets.

Example 57: The delivery system of any example herein, in particular Example 56, wherein the one or more suction ports are coupled to the elongate shaft.

Example 58: The delivery system of any example herein, in particular Example 57, wherein the elongate shaft includes an outer surface and the one or more suction ports are positioned on the outer surface.

Example 59: The delivery system of any example herein, in particular Example 58, wherein the one or more suction ports include a plurality of the suction ports circumferentially spaced from each other on the outer surface.

Example 60: The delivery system of any example herein, in particular Examples 56-59, further comprising one or more suction conduits extending along the elongate shaft and configured to provide suction for the one or more suction ports.

Example 61: The delivery system of any example herein, in particular Examples 56-60, wherein the elongate shaft includes a capsule surrounding the implant retention area.

Example 62: The delivery system of any example herein, in particular Example 61, wherein the capsule is configured to be retracted to at least partially deploy the implant from the implant retention area.

Example 63: The delivery system of any example herein, in particular Example 61 or Example 62, wherein a distal end of the capsule is positioned distal of the one or more suction ports.

Example 64: The delivery system of any example herein, in particular Examples 56-63, further comprising the implant, wherein the implant comprises a prosthetic heart valve.

Example 65: The delivery system of any example herein, in particular Example 64, wherein the prosthetic heart valve includes one or more distal anchors configured to hook over a distal tip of one of the native heart valve leaflets.

Example 66: A method comprising: applying suction from one or more suction ports to native heart valve leaflets of a native heart valve to draw the native heart valve leaflets radially inward; and at least partially deploying an implant from an elongate shaft having an implant retention area to the native heart valve with the one or more suction ports applying the suction to the native heart valve leaflets.

Example 67: The method of any example herein, in particular Example 66, wherein the one or more suction ports are coupled to the elongate shaft.

Example 68: The method of any example herein, in particular Example 67, wherein the elongate shaft includes an outer surface and the one or more suction ports are positioned on the outer surface.

Example 69: The method of any example herein, in particular Example 68, wherein the one or more suction ports include a plurality of the suction ports circumferentially spaced from each other on the outer surface.

Example 70: The method of any example herein, in particular Examples 66-69, wherein one or more suction conduits extend along the elongate shaft and provide suction for the one or more suction ports.

Example 71: The method of any example herein, in particular Examples 66-70, wherein the elongate shaft includes a capsule surrounding the implant retention area.

Example 72: The method of any example herein, in particular Example 71, further comprising retracting the capsule to at least partially deploy the implant from the implant retention area.

Example 73: The method of any example herein, in particular Example 71 or Example 72, wherein a distal end of the capsule is positioned distal of the one or more suction ports.

Example 74: The method of any example herein, in particular Examples 66-73, wherein the implant comprises a prosthetic heart valve.

Example 75: The method of any example herein, in particular Example 74, wherein the prosthetic heart valve includes one or more distal anchors configured to hook over a distal tip of one of the native heart valve leaflets.

Example 76: A prosthetic valve configured to be deployed to a native valve, the prosthetic valve comprising: a plurality of prosthetic valve leaflets; a valve body supporting the plurality of prosthetic valve leaflets; and a plurality of distal anchors each having a distal tip and configured to move from a crimped configuration to a deployed configuration, at least one of the distal tips in the crimped configuration being offset longitudinally from a position of another of the distal tips in the crimped configuration.

Example 77: The prosthetic valve of any example herein, in particular Example 76, wherein the plurality of distal anchors have a hooked shape in the deployed configuration and have an elongated shape in the crimped configuration.

Example 78: The prosthetic valve of any example herein, in particular Example 76 or Example 77, wherein the plurality of distal anchors extend radially outward from the valve body in the deployed configuration and extend axially with the valve body in the crimped configuration.

Example 79: The prosthetic valve of any example herein, in particular Examples 76-78, wherein the plurality of distal anchors are configured to hook over a distal tip of a leaflet of the native valve in the deployed configuration.

Example 80: The prosthetic valve of any example herein, in particular Examples 76-79, wherein the plurality of distal anchors are configured to be positioned within a deployment capsule of a delivery apparatus in the crimped configuration.

Example 81: The prosthetic valve of any example herein, in particular Examples 76-80, wherein a first plurality of the plurality of distal anchors have distal tips that are offset longitudinally from a position of distal tips of a second plurality of the plurality of distal anchors.

Example 82: The prosthetic valve of any example herein, in particular Example 81, wherein the first plurality of the plurality of distal anchors alternate circumferentially with the second plurality of the plurality of distal anchors.

Example 83: The prosthetic valve of any example herein, in particular Example 81, wherein the first plurality of the plurality of distal anchors are positioned adjacent to each other circumferentially and the second plurality of the plurality of distal anchors are positioned adjacent to each other circumferentially.

Example 84: The prosthetic valve of any example herein, in particular Examples 76-83, wherein the valve body includes a valve frame with a distal portion and the plurality of distal anchors each have a proximal portion coupled to the distal portion of the valve frame.

Example 85: The prosthetic valve of any example herein, in particular Example 84, wherein each of the plurality of distal anchors includes a shaft extending to the respective distal tip of the distal anchor, wherein the distal tip has a greater width than the shaft.

Example 86: A method comprising: deploying a prosthetic valve to a native valve, the prosthetic valve including: a plurality of prosthetic valve leaflets, a valve body supporting the plurality of prosthetic valve leaflets, and a plurality of distal anchors each having a distal tip and configured to move from a crimped configuration to a deployed configuration, at least one of the distal tips in the crimped configuration being offset longitudinally from a position of another of the distal tips in the crimped configuration.

Example 87: The method of any example herein, in particular Example 86, wherein the plurality of distal anchors have a hooked shape in the deployed configuration and have an elongated shape in the crimped configuration.

Example 88: The method of any example herein, in particular Example 86 or Example 87, wherein the plurality of distal anchors extend radially outward from the valve body in the deployed configuration and extend axially with the valve body in the crimped configuration.

Example 89: The method of any example herein, in particular Examples 86-88, wherein the plurality of distal anchors are configured to hook over a distal tip of a leaflet of the native valve in the deployed configuration.

Example 90: The method of any example herein, in particular Examples 86-89, wherein the plurality of distal anchors are configured to be positioned within a deployment capsule of a delivery apparatus in the crimped configuration.

Example 91: The method of any example herein, in particular Examples 86-90, wherein a first plurality of the plurality of distal anchors has distal tips that are offset longitudinally from a position of distal tips of a second plurality of the plurality of distal anchors.

Example 92: The method of any example herein, in particular Example 91, wherein the first plurality of the plurality of distal anchors alternates circumferentially with the second plurality of the plurality of distal anchors.

Example 93: The method of any example herein, in particular Example 91, wherein the first plurality of the plurality of distal anchors are positioned adjacent to each other circumferentially and the second plurality of the plurality of distal anchors are positioned adjacent to each other circumferentially.

Example 94: The method of any example herein, in particular Examples 86-93, wherein the valve body includes a valve frame with a distal portion and the plurality of distal anchors each have a proximal portion coupled to the distal portion of the valve frame.

Example 95: The method of any example herein, in particular Example 94, wherein each of the plurality of distal anchors includes a shaft extending to the respective distal tip of the distal anchor, wherein the distal tip has a greater width than the shaft.

Example 96: A prosthetic valve configured to be deployed to a native valve, the prosthetic valve comprising: a plurality of prosthetic valve leaflets; a valve body supporting the plurality of prosthetic valve leaflets; and a plurality of distal anchors each having a distal tip, at least one of the distal tips configured to have a greater diameter than another of the distal tips.

Example 97: The prosthetic valve of any example herein, in particular Example 96, wherein the plurality of distal anchors are configured to hook over a distal tip of a leaflet of the native valve, with the distal tip of each of the plurality of distal anchors being positioned radially outward of the leaflet.

Example 98: The prosthetic valve of any example herein, in particular Example 96 or Example 97, wherein the at least one of the distal tips is configured to be inflated to have a greater diameter than another of the distal tips.

Example 99: The prosthetic valve of any example herein, in particular Example 98, wherein the at least one of the distal tips is configured to be inflated with blood surrounding the at least one of the distal tips from a heart that the prosthetic valve is deployed to.

Example 100: The prosthetic valve of any example herein, in particular Example 98 or Example 99, wherein the at least one of the distal tips includes a bladder for filling the at least one of the distal tips with an inflation material.

Example 101: The prosthetic valve of any example herein, in particular Example 100, further comprising an inflation conduit for inflating the bladder with the inflation material.

Example 102: The prosthetic valve of any example herein, in particular Example 101, wherein the inflation conduit includes a proximal end coupled to the prosthetic valve, the proximal end including a valve for separably coupling to a tube for passing the inflation material into the inflation conduit.

Example 103: The prosthetic valve of any example herein, in particular Example 102, wherein the inflation conduit includes a manifold for distributing the inflation material to the plurality of distal anchors.

Example 104: The prosthetic valve of any example herein, in particular Examples 96-103, wherein a first plurality of the plurality of distal anchors have distal tips that are configured to have a greater diameter than distal tips of a second plurality of the plurality of distal anchors.

Example 105: The prosthetic valve of any example herein, in particular Example 104, wherein the first plurality are circumferentially spaced from each other, and at least one of the second plurality are positioned circumferentially between the first plurality.

Example 106: A method comprising: deploying a prosthetic valve to a native valve, the prosthetic valve including: a plurality of prosthetic valve leaflets, a valve body supporting the plurality of prosthetic valve leaflets, and a plurality of distal anchors each having a distal tip, at least one of the distal tips configured to have a greater diameter than another of the distal tips.

Example 107: The method of any example herein, in particular Example 106, wherein the plurality of distal anchors are configured to hook over a distal tip of a leaflet of the native valve, with the distal tip of each of the plurality of distal anchors being positioned radially outward of the leaflet.

Example 108: The method of any example herein, in particular Example 106 or Example 107, wherein the at least one of the distal tips is configured to be inflated to have a greater diameter than another of the distal tips.

Example 109: The method of any example herein, in particular Example 108, wherein the at least one of the distal tips is configured to be inflated with blood surrounding the at least one of the distal tips from a heart that the prosthetic valve is deployed to.

Example 110: The method of any example herein, in particular Example 108 or Example 109, wherein the at least one of the distal tips includes a bladder for filling the at least one of the distal tips with an inflation material.

Example 111: The method of any example herein, in particular Example 110, further comprising an inflation conduit for inflating the bladder with the inflation material.

Example 112: The method of any example herein, in particular Example 111, wherein the inflation conduit includes a proximal end coupled to the prosthetic valve, the proximal end including a valve for separably coupling to a tube for passing the inflation material into the inflation conduit.

Example 113: The method of any example herein, in particular Example 112, wherein the inflation conduit includes a manifold for distributing the inflation material to the plurality of distal anchors.

Example 114: The method of any example herein, in particular Examples 106-113, wherein a first plurality of the plurality of distal anchors have distal tips that are configured to have a greater diameter than distal tips of a second plurality of the plurality of distal anchors.

Example 115: The method of any example herein, in particular Example 114, wherein the first plurality are circumferentially spaced from each other, and at least one of the second plurality are positioned circumferentially between the first plurality.

Example 116: A delivery system for an implant, the delivery system comprising: an elongate shaft including an implant retention area for retaining the implant; and one or more sutures configured to couple to at least one of a plurality of distal anchors of the implant and apply a compressive force to the at least one of the plurality of distal anchors radially inward.

Example 117: The delivery system of any example herein, in particular Example 116, further comprising a plurality of the sutures, each suture configured to apply the compressive force to a respective one of the plurality of distal anchors radially inward independent of another of the plurality of distal anchors.

Example 118: The delivery system of any example herein, in particular Example 116 or Example 117, wherein the compressive force moves the at least one of the plurality of distal anchors radially inward.

Example 119: The delivery system of any example herein, in particular Examples 116-118, wherein the compressive force moves the at least one of the plurality of distal anchors from a hooked configuration towards an elongated configuration.

Example 120: The delivery system of any example herein, in particular Examples 116-119, wherein the one or more sutures are configured to form a loop extending circumferentially around the plurality of distal anchors.

Example 121: The delivery system of any example herein, in particular Examples 116-120, wherein the one or more sutures include a first portion configured to couple to the at least one of the plurality of distal anchors and a second portion configured to be retracted proximally to cause the one or more sutures to apply the compressive force to the at least one of the plurality of distal anchors radially inward.

Example 122: The delivery system of any example herein, in particular Example 121, wherein the elongate shaft includes an inner shaft configured to engage the second portion of the one or more sutures, the second portion configured to be retracted proximally through the inner shaft to cause the one or more sutures to apply the compressive force to the at least one of the plurality of distal anchors radially inward.

Example 123: The delivery system of any example herein, in particular Examples 116-122, wherein the one or more sutures include a first portion configured to couple to the at least one of the plurality of distal anchors and a second portion configured to couple to the elongate shaft, the elongate shaft configured to be retracted or rotated to cause the one or more sutures to apply the compressive force to the at least one of the plurality of distal anchors radially inward.

Example 124: The delivery system of any example herein, in particular Examples 116-123, further comprising the implant, wherein the implant comprises a prosthetic heart valve having a body including a proximal end and a distal end and a plurality of distal anchors, and the one or more sutures comprise a plurality of sutures each having a first portion configured to couple to a respective one of the plurality of distal anchors and having a second portion configured to extend proximally from the distal end of the body to the elongate shaft.

Example 125: The delivery system of any example herein, in particular Example 124, wherein the prosthetic heart valve includes a plurality of prosthetic valve leaflets and the body includes a valve frame, and the second portion is configured to pass through the valve frame to a position radially outward of the plurality of prosthetic valve leaflets.

Example 126: A method comprising: utilizing a delivery system to deploy an implant to a portion of a patient's body, the delivery system including: an elongate shaft including an implant retention area for retaining the implant, and one or more sutures configured to couple to at least one of a plurality of distal anchors of the implant and apply a compressive force to the at least one of the plurality of distal anchors radially inward.

Example 127: The method of any example herein, in particular Example 126, wherein a plurality of the sutures is configured to each apply the compressive force to a respective one of the plurality of distal anchors radially inward independent of another of the plurality of distal anchors.

Example 128: The method of any example herein, in particular Example 126 or Example 127, wherein the compressive force moves the at least one of the plurality of distal anchors radially inward.

Example 129: The method of any example herein, in particular Examples 126-128, wherein the compressive force moves the at least one of the plurality of distal anchors from a hooked configuration towards an elongated configuration.

Example 130: The method of any example herein, in particular Examples 126-129, wherein the one or more sutures form a loop extending circumferentially around the plurality of distal anchors.

Example 131: The method of any example herein, in particular Examples 126-130, wherein the one or more sutures include a first portion coupled to the at least one of the plurality of distal anchors and a second portion configured to be retracted proximally to cause the one or more sutures to apply the compressive force to the at least one of the plurality of distal anchors radially inward.

Example 132: The method of any example herein, in particular Example 131, wherein the elongate shaft includes an inner shaft engaging the second portion of the one or more sutures, the second portion configured to be retracted proximally through the inner shaft to cause the one or more sutures to apply the compressive force to the at least one of the plurality of distal anchors radially inward.

Example 133: The method of any example herein, in particular Examples 126-132, wherein the one or more sutures include a first portion coupled to the at least one of the plurality of distal anchors and a second portion coupled to the elongate shaft, the elongate shaft configured to be retracted or rotated to cause the one or more sutures to apply the compressive force to the at least one of the plurality of distal anchors radially inward.

Example 134: The method of any example herein, in particular Examples 126-133, wherein the implant comprises a prosthetic heart valve having a body including a proximal end and a distal end and a plurality of distal anchors, and the one or more sutures comprise a plurality of sutures each having a first portion coupled to a respective one of the plurality of distal anchors and having a second portion extending proximally from the distal end of the body to the elongate shaft.

Example 135: The method of any example herein, in particular Example 134, wherein the prosthetic heart valve includes a plurality of prosthetic valve leaflets and the body includes a valve frame, and the second portion passes through the valve frame to a position radially outward of the plurality of prosthetic valve leaflets.

Example 136: A delivery system for an implant, the delivery system comprising: an elongate shaft including an implant retention area for retaining the implant; and one or more sutures configured to form a loop extending circumferentially about the implant and configured to apply a compressive force to the implant radially inward.

Example 137: The delivery system of any example herein, in particular Example 136, wherein the implant comprises a prosthetic heart valve having a plurality of prosthetic valve leaflets and a body including a valve frame and a plurality of distal anchors, and the loop is configured to extend circumferentially about the body.

Example 138: The delivery system of any example herein, in particular Example 137, wherein the loop is configured to extend circumferentially around the plurality of distal anchors.

Example 139: The delivery system of any example herein, in particular Examples 136-138, wherein the implant is configured to deflect radially inward.

Example 140: The delivery system of any example herein, in particular Examples 137-139, wherein the loop is configured to extend circumferentially around the valve frame.

Example 141: The delivery system of any example herein, in particular Examples 136-140, wherein the one or more sutures include a first portion configured to form the loop about the implant and a second portion configured to be retracted proximally to cause the loop to apply the compressive force to the implant radially inward.

Example 142: The delivery system of any example herein, in particular Example 141, wherein the elongate shaft includes an inner shaft configured to engage the second portion of the one or more sutures, the second portion configured to be retracted proximally through the inner shaft to cause the loop to apply the compressive force to the implant radially inward.

Example 143: The delivery system of any example herein, in particular Examples 136-142, wherein the one or more sutures include a first portion configured to form the loop about the implant and a second portion configured to couple to the elongate shaft, the elongate shaft configured to be retracted or rotated to cause the loop to apply the compressive force to the implant radially inward.

Example 144: The delivery system of any example herein, in particular Examples 136-143, further comprising the implant comprising a prosthetic heart valve having a body including a proximal end and a distal end and a plurality of distal anchors, and the one or more sutures include a first portion configured to form the loop extending circumferentially about the implant and a second portion configured to extend proximally from the distal end of the body to the elongate shaft.

Example 145: A method comprising: utilizing a delivery system to deploy an implant to a portion of a patient's body, the delivery system including: an elongate shaft including an implant retention area retaining the implant, and one or more sutures forming a loop extending circumferentially about the implant and configured to apply a compressive force to the implant radially inward.

Example 146: The method of any example herein, in particular Example 145, wherein the implant comprises a prosthetic heart valve having a plurality of prosthetic valve leaflets and a body including a valve frame and a plurality of distal anchors, and the loop extends circumferentially about the body.

Example 147: The method of any example herein, in particular Example 146, wherein the loop extends circumferentially around the plurality of distal anchors.

Example 148: The method of any example herein, in particular Example 146, wherein the loop extends circumferentially around the valve frame.

Example 149: The method of any example herein, in particular Examples 145-148, wherein the one or more sutures include a first portion forming the loop about the implant and a second portion configured to be retracted proximally to cause the loop to apply the compressive force to the implant radially inward.

Example 150: The method of any example herein, in particular Example 149, wherein the elongate shaft includes an inner shaft engaging the second portion of the one or more sutures, the second portion configured to be retracted proximally through the inner shaft to cause the loop to apply the compressive force to the implant radially inward.

Example 151: The method of any example herein, in particular Examples 145-150, wherein the one or more sutures include a first portion forming the loop about the implant and a second portion coupled to the elongate shaft, the elongate shaft configured to be retracted or rotated to cause the loop to apply the compressive force to the implant radially inward.

Example 152: The method of any example herein, in particular Examples 145-151, wherein the implant comprises a prosthetic heart valve having a body including a proximal end and a distal end and a plurality of distal anchors, and the one or more sutures include a first portion forming the loop extending circumferentially about the implant and a second portion extending proximally from the distal end of the body to the elongate shaft.

Example 153: A delivery system for an implant, the delivery system comprising: an elongate shaft including an implant retention area for retaining the implant and an inner shaft configured to pass through the implant; and one or more sutures including a first portion configured to couple to the implant and apply a compressive force to the implant radially inward and a second portion configured to couple to the inner shaft.

Example 154: The delivery system of any example herein, in particular Example 153, wherein the implant comprises a prosthetic heart valve having a body including a valve frame and a plurality of distal anchors, and the first portion is configured to couple to the body.

Example 155: The delivery system of any example herein, in particular Example 154, wherein the first portion is configured to couple to the valve frame.

Example 156: The delivery system of any example herein, in particular Example 155, wherein the valve frame includes distal apices, and the first portion is configured to couple to at least one of the distal apices.

Example 157: The delivery system of any example herein, in particular Examples 153-156, wherein the one or more sutures are configured to form a loop extending circumferentially about the implant.

Example 158: The delivery system of any example herein, in particular Examples 153-157, wherein the second portion is configured to be retracted proximally through the inner shaft to cause the one or more sutures to apply the compressive force to the implant radially inward.

Example 159: The delivery system of any example herein, in particular Examples 153-158, wherein the second portion is configured to couple to the inner shaft such that proximal movement of the inner shaft causes the one or more sutures to apply the compressive force to the implant radially inward.

Example 160: The delivery system of any example herein, in particular Examples 153-159, wherein the second portion is configured to couple to the inner shaft such that rotation of the inner shaft causes the one or more sutures to apply the compressive force to the implant radially inward.

Example 161: The delivery system of any example herein, in particular Example 160, wherein the second portion of the one or more sutures is configured to wrap around the inner shaft when the inner shaft rotates.

Example 162: A method comprising: utilizing a delivery system to deploy an implant to a portion of a patient's body, the delivery system including: an elongate shaft including an implant retention area retaining the implant and an inner shaft configured to pass through the implant, and one or more sutures including a first portion configured to couple to the implant and apply a compressive force to the implant radially inward and a second portion configured to couple to the inner shaft.

Example 163: The method of any example herein, in particular Example 162, wherein the implant comprises a prosthetic heart valve having a body including a valve frame and a plurality of distal anchors, and the first portion is configured to couple to the body.

Example 164: The method of any example herein, in particular Example 163, wherein the first portion is coupled to the valve frame.

Example 165: The method of any example herein, in particular Example 164, wherein the valve frame includes distal apices, and the first portion is coupled to at least one of the distal apices.

Example 166: The method of any example herein, in particular Examples 162-165, wherein the one or more sutures form a loop extending circumferentially about the implant.

Example 167: The method of any example herein, in particular Examples 162-166, wherein the second portion is configured to be retracted proximally through the inner shaft to cause the one or more sutures to apply the compressive force to the implant radially inward.

Example 168: The method of any example herein, in particular Examples 162-167, wherein the second portion is coupled to the inner shaft such that proximal movement of the inner shaft causes the one or more sutures to apply the compressive force to the implant radially inward.

Example 169: The method of any example herein, in particular Examples 162-168, wherein the second portion is coupled to the inner shaft such that rotation of the inner shaft causes the one or more sutures to apply the compressive force to the implant radially inward.

Example 170: The method of any example herein, in particular Example 169, wherein the second portion of the one or more sutures is configured to wrap around the inner shaft when the inner shaft rotates.

Example 171: A prosthetic valve configured to be deployed to a native valve, the prosthetic valve comprising: a plurality of prosthetic valve leaflets; a valve body supporting the plurality of prosthetic valve leaflets; and one or more anchors each configured to anchor the prosthetic valve to the native valve and each including a first arm and a second arm and configured to extend radially outward to a tip, the tip including a loop coupling the first arm to the second arm and the first arm configured to be moved relative to the second arm to vary a distance between the first arm and the second arm.

Example 172: The prosthetic valve of any example herein, in particular Example 171, wherein each of the one or more anchors comprises a distal anchor configured to hook over a distal tip of a leaflet of the native valve.

Example 173: The prosthetic valve of any example herein, in particular Example 171 or Example 172, wherein the first arm is positioned proximal of the second arm.

Example 174: The prosthetic valve of any example herein, in particular Examples 171-173, wherein the first arm is positioned radially inward of the second arm.

Example 175: The prosthetic valve of any example herein, in particular Examples 171-174, wherein the first arm extends parallel with the second arm.

Example 176: The prosthetic valve of any example herein, in particular Examples 171-175, wherein the second arm includes a radially outward portion coupled to the loop and includes a radially inward portion comprising a free end of the second arm.

Example 177: The prosthetic valve of any example herein, in particular Example 176, wherein the free end is configured to couple to a coupler of a delivery apparatus.

Example 178: The prosthetic valve of any example herein, in particular Examples 171-175, wherein the second arm includes a radially outward portion coupled to the loop and includes a radially inward portion coupled to the valve body.

Example 179: The prosthetic valve of any example herein, in particular Examples 171-178, wherein the first arm includes a radially outward portion coupled to the loop and includes a radially inward portion coupled to the valve body.

Example 180: The prosthetic valve of any example herein, in particular Example 179, wherein the radially inward portion is integral with a flange configured to extend radially outward from the valve body.

Example 181: The prosthetic valve of any example herein, in particular Example 180, wherein the flange is configured to be positioned on a proximal side of the native valve, with the one or more anchors positioned on a distal side of the native valve.

Example 182: The prosthetic valve of any example herein, in particular Examples 179-181, wherein the valve body includes an inner frame and a sealing body having an outer frame positioned radially outward of the inner frame, and the radially inward portion of the first arm is coupled to the inner frame.

Example 183: The prosthetic valve of any example herein, in particular Examples 171-182, wherein the one or more anchors are configured to be in a deployed configuration in which the one or more anchors have a hooked shape and the tip includes the loop, and the one or more anchors are configured to be in a stretched configuration with the loop straightened and the first arm positioned proximal of the second arm.

Example 184: The prosthetic valve of any example herein, in particular Example 183, wherein each of the one or more anchors are configured to move from the stretched configuration to the deployed configuration.

Example 185: The prosthetic valve of any example herein, in particular Example 184, wherein the one or more anchors include a plurality of the anchors each configured to move from the stretched configuration to the deployed configuration independent of another one of the plurality of the anchors.

Example 186: The prosthetic valve of any example herein, in particular Example 184 or Example 185, wherein each of the one or more anchors are configured to slide relative to the valve body to move from the stretched configuration to the deployed configuration.

Example 187: The prosthetic valve of any example herein, in particular Examples 184-186, wherein each of the one or more anchors is shape set to move from the stretched configuration to the deployed configuration.

Example 188: The prosthetic valve of any example herein, in particular Examples 183-187, wherein each of the one or more anchors are configured to move from the deployed configuration to the stretched configuration.

Example 189: The prosthetic valve of any example herein, in particular Examples 183-188, wherein the first arm has a hooked shape in the deployed configuration and the second arm has a hooked shape in the deployed configuration.

Example 190: The prosthetic valve of any example herein, in particular Examples 171-189, wherein the tip includes a hinge that forms the loop.

Example 191: A method comprising: deploying a prosthetic valve to a native valve, the prosthetic valve including: a plurality of prosthetic valve leaflets, a valve body supporting the plurality of prosthetic valve leaflets, and one or more anchors each configured to anchor the prosthetic valve to the native valve and each including a first arm and a second arm and configured to extend radially outward to a tip, the tip including a loop coupling the first arm to the second arm and the first arm configured to be moved relative to the second arm to vary a distance between the first arm and the second arm.

Example 192: The method of any example herein, in particular Example 191, wherein each of the one or more anchors comprises a distal anchor configured to hook over a distal tip of a leaflet of the native valve.

Example 193: The method of any example herein, in particular Example 191 or Example 192, wherein the first arm is positioned proximal of the second arm.

Example 194: The method of any example herein, in particular Examples 191-193, wherein the first arm extends parallel with the second arm.

Example 195: The method of any example herein, in particular Examples 191-194, wherein the second arm includes a radially outward portion coupled to the loop and includes a radially inward portion comprising a free end of the second arm.

Example 196: The method of any example herein, in particular Examples 191-195, wherein the second arm includes a radially outward portion coupled to the loop and includes a radially inward portion coupled to the valve body.

Example 197: The method of any example herein, in particular Examples 191-196, wherein the first arm includes a radially outward portion coupled to the loop and includes a radially inward portion coupled to the valve body.

Example 198: The method of any example herein, in particular Example 197, wherein the radially inward portion is integral with a flange configured to extend radially outward from the valve body.

Example 199: The method of any example herein, in particular Examples 191-198, wherein the one or more anchors are configured to be in a deployed configuration in which the one or more anchors have a hooked shape and the tip includes the loop, and the one or more anchors are configured to be in a stretched configuration with the loop straightened and the first arm positioned proximal of the second arm.

Example 200: The method of any example herein, in particular Example 199, wherein each of the one or more anchors are configured to move from the stretched configuration to the deployed configuration.

Example 201: A prosthetic valve configured to be deployed to a native valve, the prosthetic valve comprising: a plurality of prosthetic valve leaflets; a valve body supporting the plurality of prosthetic valve leaflets; and one or more anchors each configured to anchor the prosthetic valve to the native valve and each configured to slide relative to the valve body.

Example 202: The prosthetic valve of any example herein, in particular Example 201, wherein each of the one or more anchors comprises a distal anchor configured to hook over a distal tip of a leaflet of the native valve.

Example 203: The prosthetic valve of any example herein, in particular Example 201 or Example 202, wherein each of the one or more anchors is configured to extend radially outward to a tip of the respective anchor.

Example 204: The prosthetic valve of any example herein, in particular Examples 201-203, wherein each of the one or more anchors is configured to have a hooked shape.

Example 205: The prosthetic valve of any example herein, in particular Examples 201-204, wherein the valve body includes a valve frame surrounding a central axis, and each of the one or more anchors is configured to slide axially relative to the valve frame.

Example 206: The prosthetic valve of any example herein, in particular Examples 201-205, wherein each of the one or more anchors is configured to slide proximally relative to the valve body.

Example 207: The prosthetic valve of any example herein, in particular Examples 201-206, wherein each of the one or more anchors is configured to slide distally relative to the valve body.

Example 208: The prosthetic valve of any example herein, in particular Examples 201-207, wherein each of the one or more anchors is configured to slide relative to the valve body to vary a distance of the one or more anchors from the valve body.

Example 209: The prosthetic valve of any example herein, in particular Examples 201-208, wherein each of the one or more anchors includes a tip and a proximal portion configured to slide along the valve body.

Example 210: The prosthetic valve of any example herein, in particular Example 209, wherein each of the one or more anchors includes a bend portion positioned between the proximal portion and the tip.

Example 211: The prosthetic valve of any example herein, in particular Example 209 or Example 210, wherein the proximal portion includes a proximal arm of the respective anchor.

Example 212: The prosthetic valve of any example herein, in particular Examples 209-211, wherein each of the one or more anchors is configured to slide relative to the valve body to vary an axial position of the tip of the respective anchor relative to the valve body.

Example 213: The prosthetic valve of any example herein, in particular Examples 201-212, further comprising a lock configured to lock a position of the one or more anchors relative to the valve body.

Example 214: The prosthetic valve of any example herein, in particular Example 213, wherein the lock comprises a ratcheting lock.

Example 215: The prosthetic valve of any example herein, in particular Examples 201-214, wherein the one or more anchors include a plurality of the anchors each configured to slide relative to the valve body independent of another one of the plurality of the anchors.

Example 216: A method comprising: deploying a prosthetic valve to a native valve, the prosthetic valve including: a plurality of prosthetic valve leaflets, a valve body supporting the plurality of prosthetic valve leaflets, and one or more anchors each configured to anchor the prosthetic valve to the native valve and each configured to slide relative to the valve body.

Example 217: The method of any example herein, in particular Example 216, wherein each of the one or more anchors comprises a distal anchor configured to hook over a distal tip of a leaflet of the native valve.

Example 218: The method of any example herein, in particular Example 216 or Example 217, wherein each of the one or more anchors is configured to have a hooked shape.

Example 219: The method of any example herein, in particular Examples 216-218, wherein the valve body includes a valve frame surrounding a central axis, and each of the one or more anchors is configured to slide axially relative to the valve frame.

Example 220: The method of any example herein, in particular Examples 216-219, wherein each of the one or more anchors is configured to slide relative to the valve body to vary a distance of the one or more anchors from the valve body.

Example 221: The method of any example herein, in particular Examples 216-220, wherein each of the one or more anchors includes a tip and a proximal portion configured to slide along the valve body.

Example 222: The method of any example herein, in particular Example 221, wherein each of the one or more anchors includes a bend portion positioned between the proximal portion and the tip.

Example 223: The method of any example herein, in particular Example 221 or Example 222, wherein each of the one or more anchors is configured to slide relative to the valve body to vary an axial position of the tip of the respective anchor relative to the valve body.

Example 224: The method of any example herein, in particular Examples 216-223, further comprising a lock configured to lock a position of the one or more anchors relative to the valve body.

Example 225: The method of any example herein, in particular Examples 216-224, wherein the one or more anchors include a plurality of the anchors each configured to slide relative to the valve body independent of another one of the plurality of the anchors.

Example 226: A delivery system for an implant, the delivery system comprising: an elongate shaft including an implant retention area for retaining the implant; and a control mechanism configured to control a deflection of at least one distal anchor of the implant independent of a deflection of at least one other distal anchor of the implant.

Example 227: The delivery system of any example herein, in particular Example 226, wherein the control mechanism includes a tether configured to control the deflection of the at least one distal anchor of the implant.

Example 228: The delivery system of any example herein, in particular Example 227, wherein the tether comprises a first tether, and the delivery system further comprises a second tether configured to control the deflection of the at least one other distal anchor of the implant.

Example 229: The delivery system of any example herein, in particular Example 227 or Example 228, wherein the tether is configured to couple to the at least one distal anchor of the implant and apply a compressive force to the at least one distal anchor radially inward.

Example 230: The delivery system of any example herein, in particular Examples 227-229, wherein a distal portion of the tether is configured to couple to the at least one distal anchor of the implant, and a proximal portion of the tether is configured to pass through the elongate shaft.

Example 231: The delivery system of any example herein, in particular Example 230, wherein the proximal portion of the tether is configured to be retracted proximally through the elongate shaft to cause the tether to apply a compressive force to the at least one distal anchor radially inward.

Example 232: The delivery system of any example herein, in particular Examples 226-231, wherein the control mechanism includes at least one actuator for controlling the deflection of the at least one distal anchor of the implant.

Example 233: The delivery system of any example herein, in particular Examples 226-232, wherein the control mechanism includes at least one actuator for controlling the deflection of the at least one other distal anchor of the implant.

Example 234: The delivery system of any example herein, in particular Examples 226-233, wherein the control mechanism includes an offset controller configured to control the deflection of at least one distal anchor of the implant simultaneously with the deflection of the at least one other distal anchor of the implant with a deflection offset between the at least one distal anchor of the implant and the at least one other distal anchor of the implant.

Example 235: The delivery system of any example herein, in particular Example 234, wherein the control mechanism includes a first drive body and a second drive body, the first drive body configured to control the deflection of at least one distal anchor of the implant, and the second drive body configured to control the deflection of the least one other distal anchor of the implant.

Example 236: The delivery system of any example herein, in particular Example 235, wherein the offset controller is configured to move the first drive body and the second drive body simultaneously.

Example 237: The delivery system of any example herein, in particular Examples 226-236, wherein the control mechanism includes an override mechanism configured to override a deflection offset between the at least one distal anchor of the implant and the at least one other distal anchor of the implant.

Example 238: The delivery system of any example herein, in particular Example 237, wherein the control mechanism includes a first tether and a second tether, the first tether being configured to control the deflection of the at least one distal anchor of the implant, and the second tether being configured to control the deflection of at least one other distal anchor of the implant, and the override mechanism moves the first tether a greater distance than the second tether.

Example 239: The delivery system of any example herein, in particular Examples 226-238, wherein the control mechanism includes a tether having a distal portion and a proximal portion, the distal portion configured to couple to the at least one distal anchor, and the proximal portion configured to couple to a drive body.

Example 240: The delivery system of any example herein, in particular Example 239, wherein the tether is configured to move the at least one distal anchor of the implant from a hooked configuration towards an elongated configuration.

Example 241: A method comprising: utilizing a delivery system to deploy an implant to a portion of a patient's body, the delivery system including: an elongate shaft including an implant retention area for retaining the implant, and a control mechanism configured to control a deflection of at least one distal anchor of the implant independent of a deflection of at least one other distal anchor of the implant.

Example 242: The method of any example herein, in particular Example 241, wherein the control mechanism includes a tether configured to control the deflection of the at least one distal anchor of the implant.

Example 243: The method of any example herein, in particular Example 242, wherein the tether comprises a first tether, and the delivery system further comprises a second tether configured to control the deflection of the at least one other distal anchor of the implant.

Example 244: The method of any example herein, in particular Example 242 or Example 243, wherein the tether is configured to couple to the at least one distal anchor of the implant and apply a compressive force to the at least one distal anchor radially inward.

Example 245: The method of any example herein, in particular Examples 241-244, wherein the control mechanism includes at least one actuator for controlling the deflection of the at least one distal anchor of the implant.

Example 246: The method of any example herein, in particular Examples 241-245, wherein the control mechanism includes an offset controller configured to control the deflection of at least one distal anchor of the implant simultaneously with the deflection of the at least one other distal anchor of the implant with a deflection offset between the at least one distal anchor of the implant and the at least one other distal anchor of the implant.

Example 247: The method of any example herein, in particular Example 246, wherein the control mechanism includes a first drive body and a second drive body, the first drive body configured to control the deflection of at least one distal anchor of the implant, and the second drive body configured to control the deflection of the least one other distal anchor of the implant.

Example 248: The method of any example herein, in particular Example 247, wherein the offset controller is configured to move the first drive body and the second drive body simultaneously.

Example 249: The method of any example herein, in particular Examples 241-248, wherein the control mechanism includes an override mechanism configured to override a deflection offset between the at least one distal anchor of the implant and the at least one other distal anchor of the implant.

Example 250: The method of any example herein, in particular Examples 241-249, wherein the control mechanism includes a tether having a distal portion and a proximal portion, the distal portion configured to couple to the at least one distal anchor, and the proximal portion configured to couple to a drive body.

Example 251: A prosthetic valve configured to be deployed to a native valve, the prosthetic valve comprising: a plurality of prosthetic valve leaflets; a valve body supporting the plurality of prosthetic valve leaflets; and one or more pacemaker leads configured to anchor the valve body in position at the native valve.

Example 252: The prosthetic valve of any example herein, in particular Example 251, wherein the one or more pacemaker leads are configured to anchor to an interior heart wall.

Example 253: The prosthetic valve of any example herein, in particular Example 252, wherein the one or more pacemaker leads include a tip configured to penetrate the interior heart wall to anchor to the interior heart wall.

Example 254: The prosthetic valve of any example herein, in particular Examples 251-253, wherein the one or more pacemaker leads are configured to extend transventricular.

Example 255: The prosthetic valve of any example herein, in particular Examples 251-254, wherein the one or more pacemaker leads are configured to anchor to a ventricular apex.

Example 256: The prosthetic valve of any example herein, in particular Examples 251-255, wherein the prosthetic valve lacks one or more anchors for anchoring to a native leaflet of the native valve.

Example 257: The prosthetic valve of any example herein, in particular Examples 251-256, wherein the one or more pacemaker leads have an adjustable length.

Example 258: The prosthetic valve of any example herein, in particular Examples 251-257, wherein at least a portion of the one or more pacemaker leads is configured to slide relative to the valve body.

Example 259: The prosthetic valve of any example herein, in particular Example 258, further comprising a lock for locking the one or more pacemaker leads in position relative to the valve body.

Example 260: The prosthetic valve of any example herein, in particular Examples 251-259, wherein a proximal portion of the one or more pacemaker leads includes an electrical terminal for coupling with a pacemaker.

Example 261: A method comprising: deploying a prosthetic valve to a native valve, the prosthetic valve including: a plurality of prosthetic valve leaflets; a valve body supporting the plurality of prosthetic valve leaflets; and one or more pacemaker leads configured to anchor the valve body in position at the native valve.

Example 262: The method of any example herein, in particular Example 261, further comprising anchoring the one or more pacemaker leads to an interior heart wall.

Example 263: The method of any example herein, in particular Example 262, wherein the one or more pacemaker leads include a tip configured to penetrate the interior heart wall to anchor to the interior heart wall.

Example 264: The method of any example herein, in particular Examples 261-263, further comprising extending the one or more pacemaker leads transventricular.

Example 265: The method of any example herein, in particular Examples 261-264, wherein the one or more pacemaker leads are configured to anchor to a ventricular apex.

Example 266: The method of any example herein, in particular Examples 261-265, wherein the prosthetic valve lacks one or more anchors for anchoring to a native leaflet of the native valve.

Example 267: The method of any example herein, in particular Examples 261-266, further comprising adjusting a length of the one or more pacemaker leads.

Example 268: The method of any example herein, in particular Examples 261-267, further comprising sliding at least a portion of the one or more pacemaker leads relative to the valve body.

Example 269: The method of any example herein, in particular Example 268, further comprising locking the one or more pacemaker leads in position relative to the valve body.

Example 270: The method of any example herein, in particular Examples 261-269, further comprising coupling a pacemaker with an electrical terminal of the one or more pacemaker leads.

Example 271: A method comprising: imaging a native heart valve; and manufacturing at least a portion of a prosthetic heart valve based on the imaging of the native heart valve.

Example 272: The method of any example herein, in particular Example 271, wherein the prosthetic heart valve includes a valve body for supporting a plurality of prosthetic heart valve leaflets, and the method comprises manufacturing at least a portion of the valve body based on the imaging of the native heart valve.

Example 273: The method of any example herein, in particular Example 272, wherein the method comprises providing a shape of at least a portion of the valve body based on an imaged shape of the native heart valve.

Example 274: The method of any example herein, in particular Example 273, wherein the shape of at least a portion of the valve body comprises a non-circular outer profile.

Example 275: The method of any example herein, in particular Examples 271-274, wherein the manufacturing includes forming at least a portion of the prosthetic heart valve upon a mandrel.

Example 276: The method of any example herein, in particular Example 275, wherein the manufacturing includes providing a shape of the mandrel based on the imaging of the native heart valve.

Example 277: The method of any example herein, in particular Example 275 or Example 276, wherein the mandrel is manufactured utilizing additive manufacturing based on the imaging of the native heart valve.

Example 278: The method of any example herein, in particular Examples 271-277, wherein the prosthetic valve includes a skirt, and the method comprises manufacturing at least a portion of the skirt based on the imaging of the native heart valve.

Example 279: The method of any example herein, in particular Examples 271-278, wherein the prosthetic heart valve includes a frame, and the method comprises manufacturing at least a portion of the frame.

Example 280: The method of any example herein, in particular Example 279, wherein the method comprises providing a shape of at least a portion of the frame based on an imaged shape of the native heart valve.

Example 281: The method of any example herein, in particular Examples 271-280, wherein the manufacturing includes providing a configuration of one or more distal anchors based on the imaging of the native heart valve.

Example 282: The method of any example herein, in particular Examples 271-281, wherein the prosthetic heart valve includes a valve body for supporting a plurality of prosthetic heart valve leaflets, the valve body including an outer valve body and an inner valve body, and the manufacturing includes forming at least a portion of the outer valve body.

Example 283: The method of any example herein, in particular Example 282, wherein the inner valve body has a circular outer profile.

Example 284: The method of any example herein, in particular Examples 271-283, wherein at least the portion of the prosthetic heart valve is manufactured to conform to a shape of the native heart valve.

Example 285: The method of any example herein, in particular Examples 271-284, wherein at least the portion of the prosthetic heart valve is manufactured on a custom basis for the particular native heart valve imaged.

Example 286: The method of any example herein, in particular Examples 271-285, wherein at least the portion of the prosthetic heart valve is manufactured utilizing a processor.

Example 287: The method of any example herein, in particular Example 286, wherein the processor is configured to determine a shape of at least the portion of the prosthetic heart valve based on the imaging of the native heart valve.

Example 288: The method of any example herein, in particular Example 286 or Example 287, wherein the processor is configured to determine whether at least the portion of the prosthetic heart valve complies with strain limits.

Example 289: The method of any example herein, in particular Examples 286-288, wherein the processor is configured to determine a shape of a mandrel based on the imaging of the native heart valve, the mandrel configured for at least a portion of the prosthetic heart valve to be formed upon.

Example 290: The method of any example herein, in particular Examples 271-289, wherein the manufacturing includes utilizing an automated fabrication assembly.

Example 291: A prosthetic heart valve configured to be deployed to a native heart valve, the prosthetic heart valve comprising: a plurality of prosthetic valve leaflets; a valve body supporting the plurality of prosthetic valve leaflets; and wherein at least a portion of the prosthetic heart valve is manufactured based on imaging of the native heart valve.

Example 292: The prosthetic heart valve of any example herein, in particular Example 291, wherein at least a portion of the valve body is manufactured based on the imaging of the native heart valve.

Example 293: The prosthetic heart valve of any example herein, in particular Example 292, wherein at least the portion of the valve body has a non-circular outer profile.

Example 294: The prosthetic heart valve of any example herein, in particular Example 292 or Example 293, wherein the valve body includes an outer valve body and an inner valve body, and at least a portion of the outer valve body is manufactured based on the imaging of the native heart valve.

Example 295: The prosthetic heart valve of any example herein, in particular Example

294, wherein the inner valve body has a circular outer profile.

Example 296: The prosthetic heart valve of any example herein, in particular Examples

291-295, wherein the prosthetic heart valve includes a skirt, and at least a portion of the skirt is manufactured based on the imaging of the native heart valve.

Example 297: The prosthetic heart valve of any example herein, in particular Examples

291-296, wherein the prosthetic heart valve includes one or more anchors, and at least a portion of the one or more anchors is manufactured based on the imaging of the native heart valve.

Example 298: The prosthetic heart valve of any example herein, in particular Examples 291-297, wherein an outer surface of the prosthetic heart valve is manufactured to contour to a shape of the native heart valve based on the imaging of the native heart valve.

Example 299: The prosthetic heart valve of any example herein, in particular Examples 291-298, wherein at least the portion of the prosthetic heart valve is manufactured on a custom basis for the particular native heart valve imaged.

Example 300: The prosthetic heart valve of any example herein, in particular Examples 291-299, wherein the prosthetic heart valve is configured to be deployed to a native mitral valve or a native tricuspid valve.

Any of the features of any of the examples, including but not limited to any of the first through 300 examples referred to above, is applicable to all other aspects and examples identified herein, including but not limited to any examples of any of the first through 300 examples referred to above. Moreover, any of the features of an example of the various examples, including but not limited to any examples of any of the first through 300 examples referred to above, is independently combinable, partly or wholly with other examples described herein in any way, e.g., one, two, or three or more examples may be combinable in whole or in part. Further, any of the features of the various examples, including but not limited to any examples of any of the first through 300 examples referred to above, may be made optional to other examples. Any example of a method can be performed by a system or apparatus of another example, and any aspect or example of a system or apparatus can be configured to perform a method of another aspect or example, including but not limited to any examples of any of the first through 300 examples referred to above.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain examples include or do not include, certain features, elements, and/or steps. Thus, such conditional language is not generally intended to imply that features, elements, and/or steps are in any way required for one or more examples.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Some examples have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, it will be recognized that any methods described herein may be practiced using any device suitable for performing the recited steps.

While a number of examples and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A system for implantation of a prosthetic valve to a native heart valve, the system comprising:
a prosthetic valve including:
a plurality of prosthetic valve leaflets,
a valve frame supporting the plurality of prosthetic valve leaflets, and
one or more anchors for anchoring the prosthetic valve to the native heart valve, wherein at least one of the anchors is axially slidable relative to the valve frame;
a delivery apparatus for delivering the prosthetic valve to the native heart valve, the delivery apparatus including an elongate shaft having a retention area for retaining the prosthetic valve; and
a control mechanism for actuating the at least one anchor to axially slide relative to the valve frame, the control mechanism including a tether for controlling retraction of the at least one anchor relative to the valve frame and a pusher shaft for controlling advancement of the at least one anchor relative to the valve frame.

2. The system of claim 1, wherein the tether is adapted to release from the at least one anchor upon deployment of the prosthetic valve to the native heart valve.

3. The system of claim 1, wherein the control mechanism includes at least one actuator operable by a user to axially slide the at least one anchor relative to the valve frame.

4. The system of claim 3, wherein the delivery apparatus includes a handle, and the at least one actuator is positioned on the handle.

5. The system of claim 1, wherein the control mechanism is operable to actuate a first one of the anchors to axially slide relative to the valve frame, and is operable to actuate a second one of the anchors to axially slide relative to the valve frame independent from the first one of the anchors.

6. The system of claim 1, wherein each of the anchors is formed with a hook shape and is adapted to extend around a downstream end of a leaflet of the native heart valve.

7. The system of claim 6, wherein each of the anchors extends radially outwardly to a tip.

8. The system of claim 1, wherein the valve frame includes an inner frame and an outer frame positioned radially outward of the inner frame.

9. The system of claim 1, wherein at least a portion of the prosthetic valve is manufactured based on imaging of the native heart valve.

10. The system of claim 1, wherein the prosthetic valve is sized for replacing a tricuspid or mitral valve.

11. A system comprising:
a prosthetic valve including:
a plurality of prosthetic valve leaflets,
a valve frame supporting the plurality of prosthetic valve leaflets, and
a plurality of anchors for anchoring the prosthetic valve to a native heart valve, wherein each of the plurality of anchors is movable relative to the valve frame; and
a control mechanism including a motor for actuating movement of the plurality of anchors relative to the valve frame, the motor adapted to actuate movement of at least one of the plurality of anchors independent of a movement of at least one other anchor of the plurality of anchors.

12. The system of claim 11, wherein the at least one of plurality the anchors is axially slidable relative to the valve frame, and the motor is adapted to axially slide the at least one of plurality the anchors relative to the valve frame.

13. The system of claim 11, further comprising a gear system adapted to be operated by the motor to actuate movement of the at least one of the plurality of anchors.

14. The system of claim 11, wherein the motor is adapted to control a deflection of the at least one of the plurality of anchors relative to the valve frame.

15. The system of claim 14, wherein the control mechanism includes an offset controller configured to control the deflection of the at least one of the plurality of anchors relative to the valve frame simultaneously with a deflection of the at least one other anchor of the plurality of anchors with a deflection offset between the at least one of the plurality of anchors and the at least one other anchor.

16. A system for implantation of a prosthetic valve to a native heart valve, the system comprising:
a prosthetic valve including:
a plurality of prosthetic valve leaflets,
a valve frame supporting the plurality of prosthetic valve leaflets, and
one or more anchors for anchoring the prosthetic valve to the native heart valve;
a delivery apparatus for delivering the prosthetic valve to the native heart valve, the delivery apparatus including an elongate shaft having a retention area for retaining the prosthetic valve; and
one or more sutures for coupling to at least one of the anchors and for adjusting a position of the at least one anchor.

17. The system of claim 16, further comprising a plurality of the sutures, each suture for moving one of the anchors of the prosthetic valve radially inward independent of another anchor of the prosthetic valve.

18. The system of claim 16, wherein the one or more sutures are shaped to form a loop extending circumferentially around the one or more anchors.

19. The system of claim 16, wherein the one or more anchors each have a distal tip, at least one of the distal tips adapted to be inflated to have a greater diameter than another of the distal tips.

20. The system of claim 16, further comprising an inflatable body adapted to inflate radially outward from the delivery apparatus for securing the delivery apparatus in a desired position.
